(19) United States
(12) Patent Application Publication
(10) Pub. No.: US 2009/0233942 A1
(43) Pub. Date: Sep. 17, 2009

(54) Genetic markers associated with response to antidepressants

(76) Inventors: Maria Athanasiou, Fond du Lac, WI (US); Kerri Holick, Brookfield, CT (US); Carol Reed, Bethany, CT (US); Benjamin Salisbury, Dayton, OH (US); Alessandro Serretti, Bologna (IT); Wei Zou, New Haven, CT (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 12/077,160

(22) Filed: Mar. 13, 2008

(51) Int. Cl. A61K 31/497; C12Q 1/68

(52) U.S. Cl. 514/254.08; 435/6

(57) ABSTRACT

Markers of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes, and their association with response to antidepressants are disclosed. Compositions and methods for detecting and using these markers in a variety of clinical applications are disclosed.

### SEQUENCE LISTING

This application contains a lengthy Sequence Listing which has been submitted via CD-R in lieu of a printed paper copy, and is hereby incorporated by reference in its entirety. The CD-R, recorded on Mar. 10, 2008, are labeled CRF, “Copy 1” and “Copy 2”, respectively. Each contains only one identical 1.48 Mb file (161531US.txt). The sequence listing was prepared in IBM-PC/MS-DOS format.

### FIELD OF THE INVENTION

This invention relates to the field of pharmacogenetics. More specifically, this invention relates to certain variants of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes, and their association with response to antidepressants.

### BACKGROUND OF THE INVENTION

Depression is an illness that occurs with a frequency of approximately five percent in the general population, and is associated with significant morbidity and mortality. Approximately one out of every thousand of the general population is admitted to a hospital annually with depression, and around three out of every thousand are referred to psychiatrists. (DEPRESSION: AN INTEGRATIVE APPROACH, Paykel, Heinemann Publ. (1989)). The core features of depressive illness include depressed mood and loss of interest or pleasure in nearly all activities. There are numerous additional symptoms which include changes in appetite or weight, sleep and psychomotor activity, loss of energy, feelings of worthlessness or guilt, difficulties in thinking, concentrating or making decisions, and recurrent thoughts of death or suicidal ideation. The diagnosis of Major Depressive Disorder (MDD) focuses on the occurrence of the core symptoms plus at least four of the additional symptoms having been present for at least two weeks. (DIAGNOSTIC AND STATISTICAL MANUAL OF MENTAL DISORDERS, 4^{th} ed., American Psychiatric Association (1994)).

Inter-individual variability in response to antidepressants is well documented, with 30-40% of depressed patients not responding to initial treatment. (Vaswani et al., Progress in Neuro-Psychopharmacol. & Biol. Psych. 27:85-102 (2003)). Evidence of a genetic component in the variability of response is seen in a study showing that the response of one family member to a particular antidepressant predicts the response of a first degree relative to that same drug. (Mancama et al., CNS Drugs 17:143-51 (2003)). Antidepressant response may be due to genetic differences in drug metabolism, in the mechanism of action of the drug, or in the disease (or disease sub-type) itself.

In the case of selective serotonin reuptake inhibitors (SSRIs), genetics has already been demonstrated to contribute to response. Specific variants of certain genes, including tryptophan hydroxylase, G protein beta 3, beta adrenergic receptor 1, angiotensin-converting enzyme, and interleukin 1 beta have been shown to have an association with treatment with SSRIs. (Serretti et al., Psychopharmcol. 174:490-503 (2004)). It would be beneficial to discover other genes which show an association with antidepressant response.

The ACE gene encodes the angiotensin I-converting enzyme and consists of 42 exons. Mattei et al. assigned the ACE gene to 17q23 by in situ hybridization (Cytogenet. Cell. Genet. 51:1041 (1989)). Angiotensin I-converting enzyme, or kinase II, is a dipeptidyl carboxypeptidase that plays an important role in blood pressure regulation and electrolyte balance by hydrolyzing angiotensin I into angiotensin II, a potent vasopressor, and aldosterone-stimulating peptide (http://www.ncbi.nlm.nih.gov/sites/entrez?db=omim).

An insertion/deletion of 287 bp has been identified in intron 16 of the ACE gene that has been demonstrated to affect response to antidepressants. Patients with one or two copies of the deletion allele showed a greater improvement in symptoms as measured by the Hamilton Depression Rating Scale (HAMD-17) after four weeks of treatment. (Baghai et al., Neurosci. Lett. 363:38-42 (2004); Bondy et al., Biol. Psych. 29:1094-1099 (2005)). In the central nervous system, the primary function of ACE is to degrade neuropeptides including substance P. Monoamine reuptake inhibitors have been shown to reduce substance P content in various rat brain regions. (Shirayama et al., Brain Res. 739:70-8 (1996); Brodin et al., Neuropharmacol. 26:581-590 (1987)), and substance P antagonists have successfully shown antidepressant efficacy in patients (Kramer et al., Science 281:1640-5 (1998)).

The ATP5G3 gene, mapped to nuclear chromosome 2q31.1, encodes the C3 (subunit 9) isoform of F₀. (Yan et al., Genomics 24(2):375-7 (1994)). F₀, the inner mitochondrial transmembrane component, conducts protons down a gradient to the F₁ component on the matrix side of the mitochondrion and the energy captured during this transition is utilized by the F₁ component to generate ATP, the ubiquitous energy storing molecule used by all cells. (Cross, Nature 427(6973):407-8 (2004)). Many neuronal functions (e.g., neurotransmitter vesicle filling, vesicle fission, and membrane coated pit formation) require ATP producing mitochondria, which often traffic to and co-localize with active synaptic regions. (Li et al., Cell 119(6):873-87 (2004)). Moreover, altered expression of ATP biosynthesis-associated genes were found in suicide victims with major depression. (Klempan et al., Mol. Psychiatry (electronic publ.) (2007)). These findings suggest that abnormalities in the mitochondrial electron transport chain may play an important role in psychiatric and neurologic diseases.

BCL2-LIKE 1 (BCL2L1, BCLXL, BCLXS), located on nuclear chromosome 20q11.21, encodes two isoforms (BCLXL and BCLXS) of the BCL-2 family via splicing and plays a role in mitochondrial function. BCLXL, which is regulated by the MAPK pathway, inhibits programmed cell death (apoptosis), whereas BCLXS promotes apoptosis. In a rat depression model, stress corresponded with decreased BCLXL expression the hippocampal CA1, CA3, and DG subfields and chronic administration of the antidepressants, reboxetine and tranylcypromine, increased BCLXL expression in the CA1 and DG subfields. (Kosten et al., Neuropsychopharmacol. (electronic publ.) (2007)). Of note, increased hippocampal apoptosis has been identified in post-mortem samples from depressed patients (Lucassen et al., Am. J. Pathol. 158(2):453-68 (2001); Lucassen et al., CNS Neurol. Disord. Drug Targets 5(5):531-46 (2006)) and BCLXL has been shown to bind the recently identified MDD susceptibility gene, APAF-1. (Harlan et al., Mol. Psychiatry 11(1):76-85 (2006)). Taken together, there appears to be a link between BCLXL and depression.

The cytochrome P450 2C9 (CYP2C9) gene located on nuclear chromosome 10q24 encodes one of the major drug metabolizing CYP450 isoforms. The CYP450 isoenzyme superfamily, predominantly present in the liver and brain to a lesser extent, catalyzes the oxidation of many drugs and chemicals. (Llerena et al., Acta Psychiatr. Scand. 87(1):23-8 (1993)). CYP2C9, as well as CYP2D6 and CYP2C19, play a role in SSRI metabolism (Brosen, Therapie 59(1):5-12 (2004)) and certain SSRIs can selectively inhibit CYP450 isoforms. Sertraline, for example can partially inhibit CYP2C9 and fluoxetine can inhibit CYP1A2, CYP2C19, CYP2D6, and CYP3A4. (Harvey et al., J. Clin. Psychopharmacol. 16(5):345-55 (1996)). The inhibition of CYP450 isoforms can not only raise SSRI concentrations, but my also raise concentrations of additional medications the patient is taking for other indications, thereby increasing the likelihood or severity of drug-drug interactions. (Matchar et al., Evid. Rep. Technol. Assess. (Full Rep.) (146):1-77 (2007)). Moreover, genetic polymorphisms have been identified in CYP450 genes that correspond to increased, partial, or deficient enzyme activity, which allow classification of these individuals as ultra rapid metabolizers (UMs), intermediate metabolizers (IMs), and poor metabolizers (PMs), respectively. (Id.). Wild-type patients are considered to be extensive metabolizers (EMs) who have normal enzyme activity. (Id.). Although a large meta-analysis focused on CYP450 polymorphisms and non-psychotic depression showed mixed results and called for larger prospective studies (Id.), a CYP2C9 polymorphism that decreases enzyme activity was observed at a significantly higher frequency in subjects with MDD compared to healthy subjects or those with schizophrenia. (Llerena et al., Pharmacogenomics J. 3(5):300-2 (2003)). This same polymorphism, in combination with a polymorphism in the serotonin transporter, 5-HTT, was later shown to increase the risk of MDD. (Dorado et al., Fundam. Clin. Pharmacol. 21(4):451-3 (2007)). Although associations between CYP450 polymorphisms and depression are still controversial, there is a definitive association between CYP450s and antidepressant metabolism.

The dopamine receptor D3 (DRD3), located on nuclear chromosome 3q13.3, encodes the D3 subclass dopamine (DA) receptor. There are two major families of dopamine receptors, D1 and D2, and DRD3 is a member of the D2 family. Both families include GPCRs and are coupled to Gₛ and Gᵢ or Gₒ proteins capable of activating or inhibiting second messenger signaling cascades, respectively. (Neve et al., J. Recept. Signal Transduct. Res. 24(3):165-205 (2004)). The modulation of these cascades leads to phosphorylation/dephosphorylation of channels and receptors that modulate cell excitability (Tseng et al., J. Neurosci. 24(22):5131-9 (2004); Wang et al., Proc. Natl. Acad. Sci. U.S.A 101(14):5093-8 (2004)), glutamate and GABA neurotransmission (Gao et al., Proc. Natl. Acad. Sci. U.S.A 98(1):295-300 (2001); Seamans et al., J. Neurosci. 21(10):3628-38 (2001)), and synaptic plasticity (Seamans et al., Prog. Neurobiol. 74(1):1-58 (2004)). For example, D2 activation can decrease NMDA and AMPA responses of prefrontal cortex pyramidal neurons (Tseng et al., supra (2004)) and D1 activation appears capable of modulating GABA release in prefrontal cortex interneurons. (Gorelova et al., J. Neurophysiol. 88(6):3150-66 (2002)). A recent meta-analysis looking for an association between decreased mood and DA levels found an association in subjects with a family history of MDD and in drug-free subjects with MDD in remission. (Ruhe et al., Mol. Psychiatry. 12(4):331-59 (2007)). However, no association between DA and decreased mood was observed in healthy subjects (Id.). These data suggest a link between depression and dopamine.

Finkel-Biskis-Jinkins (FBJ) murine osteosarcoma virus oncogene homolog, FOS (also known as c-fos) is located on nuclear chromosome 14q24.3. FOS is a major component of the AP1 transcription factor complex which includes members of the JUN family. As an immediate-early gene, c-fos expression is often used as a marker of neuronal activation because it is induced by several stimuli and the number of cells expressing c-fos is dependent on the stimulus intensity. (Lino-de-Oliveira et al., Brain Res. Bull. 55(6):747-54 (2001)). Increased c-fos expression in many areas of the brain, including the midbrain periaqueductal gray matter (PAG) region, has been shown in rat restraint (Id.) or swimming (Bellchambers et al., Neuroscience 83(2):517-24 (1998)) models of stress. Conversely, administration of the antidepressants desipramine or clomipramine decreased the level of c-fos in the PAG brain region. Moreover, administration of reboxetine or fluoxetine to unstressed rats also showed a corresponding decrease in c-fos staining in the brain (Miyata et al., Psychopharmacol. (Berl.) 177(3):289-95 (2005)). Taken together, there appears to be a link between FOS, depression and antidepressant action.

The dysbindin gene, located on nuclear chromosome 6p22.3, encodes dystrobrevin-binding-protein 1 (DTNBP1). DTNBP1 binds to the β-dystrophin protein complex, and Duchenne muscular dystrophy is caused by the absence of dystrophin. (Knuesel et al., Eur. J. Neurosci. 11 (12):4457-62 (1999)). In the brain, dysbindin is thought to play a role in synaptic function and modulate neuronal receptors (Kim et al., Prog. Neuropsychopharmacol. Biol. Psychiatry 32(2):375-9 (2008)). Indeed, dystrophin appears to be involved in the clustering and/or stabilization of GABA receptors in post-synaptic densities. (Knuesel et al., supra (1999)). Moreover, dysbindin has been shown to influence synaptic glutamate release and glutamate modulation has been associated with antidepressant response (Numakawa et al., Hum. Mol. Genet. 13(21):2699-708 (2004); Pae et al., Pharmacogenet. Genomics 17(1):69-75 (2007); Straub et al., Am. J. Hum. Genet. 71(2):337-48 (2002); Yoshimizu et al., Psychopharmacol. (Berl.) 186(4):587-93 (2006)). The effects of dysbindin and dystrophin on synaptic structure and neurotransmission suggest a contribution of this gene to the neuronal plasticity hypothesis, which claims antidepressants may work, in part, by inducing neurite sprouting. Although attempts to link DTNBP1 polymorphisms to MDD have been controversial (Kim et al., supra (2007); Zill et al., Am. J. Med. Genet. B. Neuropsychiatr. Genet. 129(1):55-8 (2004)), a recent study has demonstrated a link to antidepressant response. (Pae et al., supra (2007)).

The gamma-aminobutyric acid (GABA) A receptor gamma 3 (GABRG3) gene, located on nuclear chromosome 15q11.2, encodes the gamma-3 subunit of the GABAA receptor. GABAA is an ionotropic receptor that allows for increased Cl⁻ conductance following binding of the inhibitory neurotransmitter GABA. (Zeng et al., Brain Res. 868(2):202-14 (2000)). GABA is expressed in 10-40% of the nerve terminals located in the cerebellum, substantia nigra, and hippocampus (Hendry et al., J. Neurosci. 7(5):1503-19 (1987)), where it serves two main functions: as an inhibitory neurotransmitter and as an intermediate in energy metabolism. (Hassel et al., J. Neurochem. 71(4):1511-8 (1998)). Through its inhibitory actions, GABA plays a role in the modulation of sleep, feeding behavior, aggression, sexual behavior, pain, cardiovascular regulation, thermoregulation, locomotor activity and mood. (Paredes et al., Neurosci. Biobehav. Rev. 16(2):145-70 (1992)). With respect to mood, depression specifically, GABA transmission may play a role in neuronal plasticity, a mechanism recently implicated in antidepressant mode of action. (Ge et al., Nature 439(7076): 589-93 (2006); Hensch, Nat. Rev. Neurosci. 6(11):877-88 (2005); Overstreet-Wadiche et al., J. Neurosci. 26(8):2326-34 (2006)). Moreover, GABA levels are decreased in antidepressant-free depressed patients compared to healthy controls (Sanacora et al., Arch. Gen. Psychiatry 56(11):1043-7 (1999)) and this effect is observed in unipolar but not bipolar depressed patients. (Krystal et al., Mol. Psychiatry 7 Suppl 1S71-S80 (2002)). In animal models, depression-associated reductions in cortical GABA (Petty et al., Pharmacol. Biochem. Behav. 15(4):567-70 (1981)) could be ameliorated by antidepressant treatments, including electroconvulsive therapy. (Lloyd et al., Prog. Neuropsychopharmacol. Biol. Psychiatry 13(3-4):341-51 (1989)). In human studies of depressed patients receiving serotonin reuptake inhibitor treatment, the magnitude of treatment related rise in cortical GABA levels correlated with the degree of GABA deficit. (Krystal et al., supra (2002)). In other words, depressed patients with larger cortical GABA deficits experienced larger antidepressant-induced GABA level increases. These studies suggest a link between GABA signaling, depression and antidepressant action.

GRIA4 (GluR4), located on nuclear chromosome 11q22, encodes one of the four subunits of AMPA-type ionotropic glutamate receptors. AMPA receptors (AMPAR) are heterooligomeric receptors formed by four subunits (GluR1-4), and GRIA4 encodes the fourth subunit, GluR4. (Rosenmund et al., Science 280(5369): 1596-9 (1998)). AMPAR mediate the majority of fast excitatory synaptic transmission in the brain and are thought to be involved in both learning and memory formation. (Gomes et al., Traffic. 8(3): 259-69 (2007)). AMPAR and psychiatric disease appear to be linked. Indeed, the AMPAR subunits GluR1 and GluR4, and the AMPAR binding protein GRIP, are upregulated in the dorsolateral prefrontal cortex of elderly schizophrenics. (Dracheva et al., J. Neurosci. Res. 79(6):868-78 (2005)). Moreover, chronic (30 days) administration of the antidepressant maprotiline increased GluR1, GluR2, and GluR3 in the mouse nucleus accumbens and dorsal striatum (Tan et al., Exp. Brain Res. 170(4):448-56 (2006)), and fluoxetine has been shown to increase the phosphorylation of GluR1. (Svenningsson et al., Proc. Natl. Acad. Sci. U.S.A 99(5):3182-7 (2002)). These studies suggest a link between AMPA receptors and antidepressants.

The Laminin alpha 4 gene (LAMA4), located on nuclear chromosome 6q21, encodes the laminin A chain. Laminin, a constituent of the basement membrane is composed of 3 non-identical chains (A, B1, and B2). Laminin, the phosphorylated form of cAMP response element binding protein (pCREB), and cell adhesion molecule L1 (CAM-L1) and all appear to play a role in neuronal plasticity. CREB can affect the regulation of CAM-L1 transcription (Crossin et al., Dev. Dyn. 218(2):260-79 (2000)) and the heterophilic binding of CAM-L1 and laminin initiates neuronal plasticity. (Hall et al., J. Neurochem. 75(1):336-46 (2000)). Moreover, laminin, pCREB, and CAM-L1 are modulated by stress and antidepressants (Laifenfeld et al., Neurobiol. Dis. 20(2):432-41 (2005)). Laminin, pCREB, and CAM-L1 were decreased in the rat hippocampus and frontal cortex following a 6 week exposure to chronic stress. (Id.). Conversely, chronic administration of the antidepressant desipramine to unstressed rats increased laminin and CAM-L1 in both the hippocampus and frontal cortex, and increased pCREB in the frontal cortex only. (Id.). In a human study decreased laminin was found in post-mortem cortex samples from depressed, bipolar, and schizophrenic patients and decreased CAM-L1 was found in cortical samples from depressed and schizophrenic patients (Laifenfeld et al., Biol. Psychiatry 57(7):716-25 (2005)). Moreover, the same study found cortical laminin and CAM-L1 levels were lower in unmedicated depressed subjects when compared to medicated depressed subjects or control subjects (Id.). These studies suggest that laminin, pCREB, and CAM-L1 may play roles in depression and the mode of antidepressant action.

Mitogen-activated protein kinase 1 (MAPK1 or ERK2), located on nuclear chromosome 22q11.2, is involved in MAPK signal transduction, a widely used signaling cascade. In a postmortem study of suicide subjects, MAPK1 activity and expression was significantly decreased in the hippocampus whereas no change was observed in the cerebellum when compared to non-psychiatric control subjects. (Dwivedi et al., J. Neurochem. 77(3):916-28 (2001)). In a more recent study, downstream targets of MAPK1, BDNF (brain-derived neurotrophic factor) and NTF3 (neurotrophin 3), were decreased in the hippocampus of depressed suicide vs. non-depressed/non-psychotic non-suicide subjects. (Karege et al., Brain Res. Mol. Brain Res. 136(1-2):29-37 (2005)). Conversely, antidepressants appear to have the opposite effect. Addition of the antidepressant fluoxetine to cultured rat astrocytes increased MAPK1 activation (Mercier et al., J. Mol. Neurosci. 24(2):207-16 (2004)), and the mood stabilizers lithium and valproate increase the activity of the MAPK pathway in rat hippocampus and frontal cortex (Einat et al., J. Neurosci. 23(19):7311-6 (2003)). These data suggest that antidepressant treatment may ameliorate depression-associated reduction in the activity of the MAPK pathway (Karege et al., supra (2005)).

The neuropeptide Y receptor Y1 (NPY1R), located on nuclear chromosome 4q31.2, is a receptor for neuropeptide Y (NPY), a 36-amino acid peptide widely distributed in the central and peripheral nervous system. In rat brain, NPY binding to NPY1R was strongly dependent on PLC (phospholipase C) activity and this binding could be inhibited by PLC inhibitors. (Parker et al., J. Pharmacol. Exp. Ther. 286(1):382-91 (1998)). Intracerebroventricular injection of NPY exerted dose dependent antidepressant-like effect in mice and this effect may act through 5-HT (serotonin) and NA (noradrenaline) neurotransmission. (Redrobe et al., Neuropsychopharmacol. 26(5):615-24 (2002)). NPY is co-localized with 5-HT-ergic and NA-ergic neurons and modulates the release of 5-HT and NA. (Finta et al., Naunyn. Schmiedebergs Arch. Pharmacol. 346(4):472-4 (1992); Martire et al., Eur. J. Pharmacol. 230(2):231-4 (1993); Schlicker et al., Naunyn. Schmiedebergs Arch. Pharmacol. 343(2):117-22 (1991)). Moreover, administration of NPY1R agonist increased levels of both 5-HT and NA in the rodent brain (Hastings et al., Brain Res. 750(1-2):301-4 (1997); Song et al., Brain Behav. Immun. 10(1):1-16 (1996)) and reduced anxiety. (Sorensen et al., J. Neurosci. Res. 77(5):723-9 (2004)). Several reports suggest NPY may be acting through NPY1R specifically to induce antidepressant-like effects. Indeed, NPY1R specific antagonists blocked the antidepressant effects of either intracerebroventricular NPY injection (Redrobe et al., Neuropsychopharmacol. 26(5): 615-24 (2002)) or hippocampal C3 region injection of NPY (Ishida et al., Hippocampus 17(4):271-80 (2007)). These studies suggest a link between depression and NPYR1.

Opioid receptor delta (OPRD1) is an endorphin receptor located on nuclear chromosome 1p36. Opioids affect biological function through three main types of opioid G protein-coupled receptors termed mu- (endorphins), delta- (enkephalinis) and kappa- (dynorphins). (Mansour et al., Trends Neurosci. 18(1):22-9 (1995)). In contrast to OPRM1 knockout mice, mice lacking OPRD1 exhibit increased locomotor activity, increased anxiety and increase depressive-like behaviors. (Filliol et al., Nat. Genet. 25(2):195-200 (2000)). Although no human studies linking OPRD1 and depression exist to our knowledge, polymorphisms in OPRD1 have been associated with substance abuse (Zhang, Mol. Psychiatry (electronic publ.) (2007)) suggesting a potential link with psychiatric function.

Opioid receptor mu (OPRM1) is an endorphin receptor located on nuclear chromosome 6q24. Opioids affect biological function through three main types of opioid G protein-coupled receptors termed mu- (endorphins), delta- (enkephalinis) and kappa- (dynorphins). (Mansour et al., supra (1995)). OPRM1-deficient mice exhibit decreased anxiety (Yoo et al., Synapse 54(2):72-82 (2004b)), less depressive-like behavior (Filliol et al., Nat. Genet. 25(2):195-200 (2000); Yoo et al., supra (2004)), and increased 5-HT_{1A} expression, suggesting a possible a compensatory mechanism for decreased opioid neurotransmission. (Yoo et al., supra (2004)). A recent human study demonstrated that decreased mu-opioid neurotransmission in healthy controls when instructed to think of events causing sadness (sad state). In contrast, female MDD patients under the same conditions exhibited increased mu-opioid system neurotransmission. (Kennedy et al., Arch. Gen. Psychiatry 63(11):1199-208 (2006)). Moreover, MDD patients who were subsequently shown to respond to a ten-week course of SSRI, exhibited increased mu-opioid activation during the sad state, whereas non-responders exhibited decreased activation. (Kennedy et al., supra (2006)). These data suggest OPRM1 may be over-activated in patients with MDD, particularly in patients responsive to SSRIs. (Kennedy et al., supra (2006)).

The PER3 encodes the homolog of Drosophila Period 3. It maps to chromosome 1p36.23 and consists of 18 exons. Period 3 is a clock gene involved in mammalian circadian rhythms, which have been shown to be disrupted in major depressive disorder. (Bunney et al., Neuropsychopharm. 22(4):335-45 (2000)). Polymorphisms, particularly 1940T>G (amino acid change V647G), in PER3 have been found to be associated with delayed sleep phase syndrome. (Ebisawa et al., EMBO Reports 2(4):342-6 (2001)). Polymorphisms in PER3 have been recently associated with response to SSRI treatment and circadian changes in mood symptoms (Artioli et al., Neuropsychopharm. 17(9):587-94 (2007)). Given the effect of these polymorphisms on sleep cycles and the role of sleep disturbances in major depression, it is rational that variants of PER3 may be associated with major depression or its treatment.

Phospholipase C-β1 (PLCB1), located on nuclear chromosome 20p12, generates 1,4,5-trisphosphate (IP3) and diacylglycerol (DAG) from phosphatidylinositol 4,5-bisphosphate (IP2). This conversion is key to the intracellular transduction of many extracellular signals. Various neurotransmitter receptors, such as the histamine H₁, serotonin 5-HT₂, α adrenergic and type 1 metabotropic glutamate receptors activate PLC-β1. (Cockcroft et al., Biochem. J. 288 (Pt. 1):1-14 (1992); Lucas et al., Trends Pharmacol. Sci. 16(7):246-52 (1995); Pin et al., Neuropharmacology 34(1): 1-26 (1995); Rhee et al., J. Biol. Chem. 267(18):12393-6 (1992)). PLC-β1 knockout mice exhibit schizophrenic-like behaviors (e.g., hyper-locomotion, deficient sensorimotor gating, reduced startle responses, etc.) that can be rescued by administration of the antipsychotics clozapine (McOmish et al., Mol. Psychiatry (electronic publ.) (2007)) or haloperidol. (Koh et al., Genes Brain Behav. 7(1):120-8 (2008)). In a study of post-mortem cortex from human suicide victims with depression, PLC-β1 expression was decreased compared to normal subjects (Pandey, Am. J. Psychiatry 156(12):1895-901 (1999)). Phospholipase therefore appears to be associated with psychiatric disease.

The PSMD1 gene encodes proteasome (prosome, macropain) 26S subunit, non-ATPase, 1. The gene consists of 25 exons and maps to chromosome 2q37.1. The 26S proteasome is a multicatalytic proteinase complex with a highly ordered structure composed of 2 complexes, a 20S core and a 19S regulator. Proteasomes are distributed throughout eukaryotic cells at a high concentration and cleave peptides in an ATP/ubiquitin-dependent process in a non-lysosomal pathway. An essential function of a modified proteasome, the immunoproteasome, is the processing of class I MHC peptides. This gene encodes the largest non-ATPase subunit of the 19S regulator lid, which is responsible for substrate recognition and binding.

Function of the 26S proteasome has been shown to be inhibited by prostaglandin J2 (PGJ2), an inflammatory by-product of the cyclooxygenase-2 pathway. (Wang et al., J. Biol. Chem. 281:21377-86 (2006)). A role of PGJ2 has been proposed in neurodegenerative disorders, which are characterized by aggregatation of ubiquitinated proteins due to lack of proteasome degradation. In addition, there is also a long history of research into the role of inflammation in psychiatric disorders including the Nobel Prize winning work of Julius Wagner-Jauregg in 1927. Specifically, cytokine hypersecretion has been associated with major depression. (Connor et al., Life Sci. 62:583-606 (1998)). Therefore, although not directly investigated to date, these observations suggest a potential role of PSMD1 in major depression and its treatment via an inflammatory mechanism not yet determined.

Within intron 16 of the PSMD1 gene, resides the HTR2B gene. The HTR2B gene codes the serotonin 5-HT2B receptor and consists of four exons. Le Coniat et al. mapped it to 2q36.3-q37.1 by fluorescence in situ hybridization (Genomics 32:172-73 (1996)).

Approximately fourteen receptor subtypes for serotonin have been identified that have various physiologic functions. (Barnes et al., Neuropharmacol. 38:1083-1152 (1991)). The 5-HT2 receptors mediate many of the central and peripheral physiologic functions of serotonin. Cardiovascular effects include contraction of blood vessels and shape changes in platelets; central nervous system effects include neuronal sensitization to tactile stimuli and mediation of hallucinogenic effects of phenylisopropylamine hallucinogens. (http://www.ncbi.nlm.nih.gov/entrez/dispomim.cgi?cmd=entry&id=601122).

Studies have identified polymorphisms in the HTR2B gene to be associated with drug abuse (Lin et al., Pharmacogenetics 14(12):805-11 (2004)) and early-onset obsessive compulsive disorder. (Kim et al., Mol. Cell. Probes 14(1):47-52 (2000)). 5-HT2B receptor activation leads to phosphorylation of the serotonin transporter thereby impairing its function as measured by decreased transport velocity and Na⁺, K⁺-ATPase activity (Launay et al., FASEB J. 20:1843-54 (2006)). Modulation of serotonin transporter function by the 5-HT2B receptor would suggest a potential role of this receptor subtype in mediating the effects of serotonin reuptake inhibitors. Moreover, 5-HT2B agonism can augment the anxiolytic-like properties of the SSRI antidepressant paroxetine. (Nic Dhonnchadha et al., Psychopharmacol. (Berl.) 179(2): 418-29 (2005)). Moreover, an oral melatonin MT1/MT2 agonist with 5-HT2B/5-HT2C antagonist activity is currently is currently being developed for the treatment of MDD (Dubocovich, Curr. Opin. Investig. Drugs 7(7):670-80 (2006)). Taken together, these studies suggest 5-HT2B plays a role in depression.

The Spectrin SH3 Domain-Binding Protein 1 (also known as ABL Interactor 1 (ABI1)) gene is found in nuclear chromosome 10p11.2. ABI1 contains SH3 and proline rich domains which are involved in binding to c-Ab1, a nonreceptor tyrosine kinase (NRTK) implicated in cell growth, apoptosis, and leukemia (c-Ab1 is often fused to BCR in many cases of myeloid leukemia). ABI1, normally highly expressed in the brain (Shi et al., Genes Dev. 9(21):2583-97 (1995)), is differentially post-translationally modified during CNS maturation. (Courtney et al., Mol. Cell. Neurosci. 16(3):244-57 (2000)). In addition, ABI1 has been linked to cytoskeletal reorganization through its interactions with Sos1 and Eps8, a substrate complex of receptor tyrosine kinases, such as EGFR (epidermal growth factor receptor). (Scita et al., Nature 401(6750):290-3 (1999)). Actin, a major component of dendritic spines (Fifkova et al., J. Cell. Biol. 95(1):345-50 (1982)), is involved in stable long term potentiation (Krucker et al., Proc. Natl. Acad. Sci. U.S.A 97(12):6856-61 (2000)), and dynamic rearrangements of the actin cytoskeleton are required for normal presynaptic function. (Kim et al., J. Neurosci. 19(11):4314-24 (1999)). It has therefore been suggested that c-Ab1, and its target ABI1 may play a role in neuronal development and plasticity. (Moresco et al., J. Neurophysiol. 89(3):1678-87 (2003)). Given that antidepressants may function by altering neuronal plasticity, ABI1 may be linked to antidepressant response.

LOC402382 is a predicted gene, similar to collagen, type I, alpha 2 (COL1A2)). Although there is no known function for this gene, the homolgous COLIA2 gene, normally expressed in the skin, tendon and bone (Savaraj et al., Cancer Invest. 23(7):577-81 (2005)), was found to be overexpressed in human medulloblastomas (Liang et al., J. Neurooncol. 86(2):133-41 (2007)). Medulloblastomas are malignant and invasive embryonal tumors that typically present in the cerebellum of children. By comparing medulloblastoma tissue to normal cerebellum via microarray, the authors observed an increase in COL1A2 expression in the tumor (Liang et al., supra (2007)). Moreover, they hypothesize a receptor for type 1 collagen, the β1 subunit of integrin, may be incorrectly signaling due to the overexpression of COL1A2 and that this signaling is misregulating cell survival and proliferation (Liang et al., supra (2007)). Although a link between LOC402382 and depression/antidepressants remains to be found, the homologous gene, COLIA2, has been found to be misregulated in the pathogenenic brain.

The neurocalcin delta (NCALD) gene, found on nuclear chromosome 8q22.2, encodes a member of the EF-hand calcium-binding protein superfamily. NCALD, predominantly expressed in retinal photoreceptors and neurons, (Wang et al., Biochim. Biophys. Acta 1518(1-2):162-7 (2001)), contains two pairs of EF-hand calcium-binding loop sequences and a N-terminal myristoylation signal. (Vijay-Kumar et al., Nat. Struct. Biol. 6(1):80-8 (1999)). Although the cellular function of NCALD is largely unknown, it has been suggested to regulate cGMP levels via a direct interaction with membrane bound guanylyl cyclase. (Duda et al., Mol. Cell Biochem. 267(1-2):107-22 (2004); Krishnan et al., Biochemistry 43(10):2708-23 (2004)). Moreover, NCALD may play a role in neurotransmitter release since it is know to bind clathrin-coated vesicles. (Ivings et al., Biochem. J. 363(Pt. 3):599-608 (2002)). Although polymorphisms in NCALD have been associated with susceptibility of diabetic nephropathy (Kamiyama et al., Hum. Genet. 122(3-4):397-407 (2007)), and decreased NCALD is found the prefrontal cortex of a rat model of schizophrenia (Vercauteren et al., Proteomics 7(19): 3569-79 (2007)), no direct associations have been made between depression and NCALD. However, given the association between NCALD and neurotransmitter release, it is plausible to consider that a link between NCALD and depression/antidepressant action may exist.

### SUMMARY OF THE INVENTION

Accordingly, the inventors herein have discovered markers in the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes, that are associated with response to antidepressants. These markers have a variety of pharmacogenetic research and clinical applications.

In a first aspect, the invention provides a method for predicting whether an individual will respond to an antidepressant comprising determining the presence or absence in the individual of a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker, and making a prediction based on the results, wherein if the marker is present, then the prediction is that the individual is likely to respond to the antidepressant and if the marker is absent, the prediction is that the individual is not likely to respond to antidepressant.

In another aspect, the invention provides a method for treating depression in an individual comprising determining the presence or absence in the individual of a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker, and making a treatment decision based on the results, wherein if the marker is present, then the decision is to prescribe to the individual the lowest approved dose of an antidepressant, and if the marker is absent, then the decision is to either prescribe to the individual the antidepressant at a dose that is higher than the lowest approved dose, or prescribe to the individual a therapy not including the antidepressant that is effective in treating depression.

In another aspect, the invention provides a kit for detecting a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker comprising a set of one or more oligonucleotides designed for identifying each of the alleles at each polymorphic site in the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker.

### Sequence Listings

SEQ ID NO:1 illustrates a reference sequence for the ACE gene.

SEQ ID NO:2 illustrates a reference sequence for the ATP5C3 gene.

SEQ ID NO:3 illustrates a reference sequence for the BCL2L1 gene.

SEQ ID NO:4 illustrates a reference sequence for the CYP2C9 gene.

SEQ ID NO:5 illustrates a reference sequence for the DRD3 gene.

SEQ ID NO:6 illustrates a reference sequence for the FOS gene.

SEQ ID NO:7 illustrates a reference sequence for the DTNBP1 gene.

SEQ ID NO:8 illustrates a reference sequence for the GABRG3 gene.

SEQ ID NO:9 illustrates a reference sequence for the GRIA4 gene.

SEQ ID NO:10 illustrates a reference sequence for the LAMA4 gene.

SEQ ID NO:11 illustrates a reference sequence for the MAPK1 gene.

SEQ ID NO:12 illustrates a reference sequence for the NPY1R gene.

SEQ ID NO:13 illustrates a reference sequence for the OPRD1 gene.

SEQ ID NO:14 illustrates a reference sequence for the OPRM1 gene.

SEQ ID NO:15 illustrates a reference sequence for the PER3 gene.

SEQ ID NO:16 illustrates a reference sequence for the PLCB1 gene.

SEQ ID NO:17 illustrates a reference sequence for the PSMD1 gene.

SEQ ID NOs:18-20 each illustrate a reference sequence for the ABI1 gene.

SEQ ID NO:21 illustrates a reference sequence for the LOC402382 gene.

SEQ ID NO:22 illustrates a reference sequence for the NCALD gene.

### DETAILED DESCRIPTION OF THE INVENTION

### I. Definitions

So that the invention may be more readily understood, certain terms are first defined.

As used in the specification and the claims, “a” or “an” means one or more unless explicitly stated otherwise. As used herein, “another” means at least a second or more.

Throughout this specification, the word “comprise” or variations such as “comprises” or “comprising” will be understood to imply the inclusion of a stated integer or groups of integers but not the exclusion of any other integer or group of integers.

“Allele” is a particular form of a gene or other genetic locus, distinguished from other forms by its particular nucleotide sequence, the term allele also includes one of the alternative polymorphisms (e.g., a SNP) found at a polymorphic site. In some contexts, it will be readily apparent to the skilled artisan that the term allele refers to the form of a locus that is present on a single chromosome in a somatic cell obtained from an individual; if the locus is on an autosomal chromosome, then the somatic cell in the individual will normally have two alleles for the locus. If these alleles have identical sequences, the individual is homozygous for that locus, and if the two alleles have different sequences, then the individual is heterozygous for the locus. If the locus is on a sex chromosome, then somatic cells from female individuals normally have two alleles, which may have the same or different sequences, while somatic cells from male individuals normally only has one allele for the locus.

“Antidepressant” is intended to refer to any drug useful in treating depression. It can include an SSRI such as vilazodone, fluoxetine, paroxetine, escitalopram, citalopram, and sertraline; a serotonin-norepinephrine reuptake inhibitor (SNRI) such as mirtazapine; a norepinephrine (noradrenaline) reuptake inhibitor (NRI) such as reboxetine; a norepinephrine-dopamine reuptake inhibitor such as bupropion; a tricyclic antidepressant (TCA) such as amitriptyline and desipramine; and a monoamine oxidase inhibitor (MAOI) such as phenelzine moclobemide selegiline.

“Disease” refers to an interruption, cessation, or disorder of one or more body functions, structures, systems or organs.

“Drug” includes any therapeutic or prophylactic compound, substance or agent including, without limitation, a small molecule, protein, vaccine, antibody or nucleic acid. In the description herein of some embodiments of the invention, it will be evident to the skilled artisan that the term drug can include a pharmaceutical composition or drug product comprising a therapeutic or prophylactic compound, substance or agent.

“Gene” is a segment of DNA that contains the coding sequence for a protein, wherein the segment may include promoters, exons, introns, and other untranslated regions that control expression.

“Marker” in the context of the present invention is a specific copy number of a specific polymorphism of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD genes that is associated with response to antidepressants. Preferred ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD markers are those shown in Tables A-1 through A-20, respectively (Appendix A), as well as genetic markers that are highly correlated with any marker in Tables A-1 through A-20, respectively (Appendix A) and/or are replaced by the same copy number of a substitute polymorphism, each of which is referred to herein as an alternate genetic marker. A substitute polymorphism comprises a sequence that is similar to that of any of the markers shown in Tables A-1 through A-20 (Appendix A), but in which the allele at one or more of the specifically identified polymorphic sites in that marker has been substituted with the allele at a different polymorphic site, whose substituting allele is in high linkage disequilibrium (LD) with the allele at the specifically identified polymorphic site. A linked polymorphism is any type of polymorphism, including a haplotype, which is in high LD with any one of the markers shown in Tables A-1 through A-20 (Appendix A). Two particular alleles at different loci on the same chromosome are said to be in LD if the presence of one of the alleles at one locus tends to predict the presence of the other allele at the other locus. Alternate genetic markers, which are further described below, may comprise types of variations other than SNPs, such as indels, RFLPs, repeats, etc.

“Genotype” is an unphased 5′ to 3′ sequence of the two alleles, typically a nucleotide pair, found at a set of one or more polymorphic sites in a locus on a pair of homologous chromosomes in an individual.

“Genotyping” is a process for determining a genotype of an individual.

“Haplotype pair” refers to the two haplotypes found for a locus in a single individual.

“Haplotyping” refers to any process for determining one or more haplotypes in an individual, including the haplotype pair for a particular set of PS, and includes use of family pedigrees, molecular techniques and/or statistical inference.

“Isolated” is typically used to reflect the purification status of a biological molecule such as RNA, DNA, oligonucleotide, or protein, and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with the methods of the present invention.

“Locus” refers to a location on a chromosome or DNA molecule corresponding to a gene, a physical feature such as a polymorphic site, or a location associated with a phenotypic feature.

“Nucleotide pair” is the set of two nucleotides (which may be the same or different) found at a polymorphic site on the two copies of a chromosome from an individual.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length. The sequence of an oligonucleotide can be designed to specifically hybridize to any of the allelic forms of a locus; such oligonucleotides are referred to as allele-specific probes. If the locus is a PS comprising a SNP, the complementary allele for that SNP can occur at any position within an allele-specific probe. Other oligonucleotides useful in practicing the invention specifically hybridize to a target region adjacent to a PS with their 3′ terminus located one to less than or equal to about 10 nucleotides from the PS, preferably ≦about 5 nucleotides. Such oligonucleotides hybridizing adjacent to a PS are useful in polymerase-mediated primer extension methods and are referred to herein as “primer-extension oligonucleotides.” In a preferred embodiment, the 3′-terminus of a primer-extension oligonucleotide is a deoxynucleotide complementary to the nucleotide located immediately adjacent to the PS.

“Phased sequence” refers to the combination of nucleotides present on a single chromosome at a set of polymorphic sites, in contrast to an unphased sequence, which is typically used to refer to the sequence of nucleotide pairs found at the same set of PS in both chromosomes.

“Polymorphic site” or “PS” refers to the position in a genetic locus or gene at which a SNP or other nonhaplotype polymorphism occurs. A PS is usually preceded by and followed by highly conserved sequences in the population of interest and thus the location of a PS is typically made in reference to a consensus nucleic acid sequence of thirty to sixty nucleotides that bracket the PS, which in the case of a SNP polymorphism is sometimes referred to as a context sequence for the SNP. The location of the PS may also be identified by its location in a consensus or reference sequence relative to the initiation codon (ATG) for protein translation. The skilled artisan understands that the location of a particular PS may not occur at precisely the same position in a reference or context sequence in each individual in a population of interest due to the presence of one or more insertions or deletions in that individual as compared to the consensus or reference sequence. Moreover, it is routine for the skilled artisan to design robust, specific and accurate assays for detecting the alternative alleles at a polymorphic site in any given individual, when the skilled artisan is provided with the identity of the alternative alleles at the PS to be detected and one or both of a reference sequence or context sequence in which the PS occurs. Thus, the skilled artisan will understand that specifying the location of any PS described herein by reference to a particular position in a reference or context sequence (or with respect to an initiation codon in such a sequence) is merely for convenience and that any specifically enumerated nucleotide position literally includes whatever nucleotide position the same PS is actually located at in the same locus in any individual being tested for the presence or absence of a genetic marker of the invention using any of the genotyping methods described herein or other genotyping methods well-known in the art.

“Polymorphism” refers to one of two or more genetically determined alternative sequences or alleles that occur for a gene or a genetic locus in a population. As used herein, the term polymorphism includes, but is not limited to (a) a sequence of as few as one nucleotide that occurs at a polymorphic site (as defined above), which is also referred to herein as a single nucleotide polymorphism (SNP) and (b) a sequence of nucleotides that occur on a single chromosome at a set of two or more polymorphic sites in the gene or genetic locus of interest, which is also referred to herein as a haplotype. The different alleles of a polymorphism typically occur in a population at different frequencies, with the allele occurring most frequently in a selected population sometimes referenced as the “major” or “wildtype” allele. Diploid organisms may be homozygous or heterozygous for the different alleles that exist. A biallelic polymorphism has two alleles, and the minor allele may occur at any frequency greater than zero and less than 50% in a selected population, including frequencies of between 1% and 2%, 2% and 10%, 10% and 20%, 20% and 30%, etc. A triallelic polymorphism has three alleles. In addition to SNPs and haplotypes, examples of polymorphisms include restriction fragment length polymorphisms (RFLPs), variable number of tandem repeats (VNTRs), dinucleotide repeats, trinucleotide repeats, tetranucleotide repeats, simple sequence repeats, insertion elements such as Alu, and deletions of one or more nucleotides.

“Treat” means to administer a drug internally or externally to a patient having one or more disease symptoms for which the drug has known therapeutic activity. Typically, the drug is administered in an amount effective to alleviate one or more disease symptoms in the treated patient or population, whether by inducing the regression of or inhibiting the progression of such symptom(s) by any clinically measurable degree. The amount of a drug that is effective to alleviate any particular disease symptom (also referred to as the “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every patient, it should alleviate the target disease symptom(s) in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Antidepressant response” or “respond to an antidepressant” or “response to an antidepressant” is intended to refer to the change in an individual's depressive symptoms following antidepressant treatment/administration, preferably as measured by change in the score on any of the Montgomery-Asberg Depression Rating Scale (MADRS) (Montgomery et al., Br. J. Psychiatry 134:382-9 (1979)), all 21 items of the Hamilton Depression Rating Scale (HAM-D-21) (Hamilton, J. Neurol. Neurosurg. Psychiat. 23:56-61 (1960)), the first 17 items of the Hamilton Depression Rating Scale (HAM-D-17) (Id.), the Hamilton Anxiety Scale (HAM-A) (Hamilton, Br. J. Med. Psychol. 32(1):50-5 (1959)), and the Clinical Global Impression Improvement scale (CGI-I). (NATIONAL INSTITUTE OF MENTAL HEALTH, Ecdeu, ed. (1976)). The MADRS is an observer rating scale that has proven to be an efficient and practical measure of depression that is sensitive to treatment effects. The HAM-D-21 is a 21-item Likert scale used for the rating of depressive symptom severity in patients who have already been confirmed as meeting this diagnosis. Its psychometric properties are well understood and correlations between score and level of symptom severity are documented. The HAM-D-17 is an assessment of the first 17 items from the 21-item scale. The CGI-I measures change from the baseline state at every subsequent visit providing a global evaluation of the patient's improvement over time. The HAM-A is a 14-item rating scale developed to quantify the severity of anxiety.

With regard to all these depression rating scales, the lower the score, the lower the presence of depressive symptoms. Thus, a greater downward change in any of these rating scales following antidepressant treatment/administration when compared to patients given the antidepressant or a placebo indicates a “good” or “positive” or “better” response to the antidepressant (or, simply, “response”).

### II. Composition and Phenotypic Effect of Markers of Antidepressant Response

As described above and in the examples below, genetic markers according to the present invention are associated with response to antidepressants, and are referred to herein as ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD markers. Each marker of the invention is a combination of a particular polymorphism associated with the antidepressant response and a copy number of that polymorphism. Preferably, the polymorphism is one of the markers shown in Appendix A, each of which contains a sequence for a specific set of PSs in the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD genes. The locations of these marker PSs in the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes are at positions corresponding to those identified in Tables A-21 through A-40 in Appendix A. In describing the PSs in the markers of the invention, reference is made to the sense strand of a gene for convenience. However, as recognized by the skilled artisan, nucleic acid molecules containing a particular gene may be complementary double stranded molecules and thus reference to a particular site on the sense strand refers as well to the corresponding site on the complementary antisense strand.

As described in more detail in the examples below, the genetic markers of the invention are based on the discovery by the inventors of associations between particular copy numbers of certain polymorphisms in the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes and antidepressant response. Individuals having the copy number indicated for each of the polymorphisms shown in Appendix A were more likely to respond to an antidepressant relative to individuals having other copy numbers of those polymorphisms. Moreover, as shown in Tables 1-20 below, the association between the presence of these genetic markers and response to antidepressants is statistically significant.

In addition, the skilled artisan will appreciate that all of the embodiments of the invention described herein may frequently be practiced using an alternate genetic marker for any of the genetic markers in Tables A-1 through A-20 (Appendix A). Alternate genetic markers are readily identified by determining the degree of linkage disequilibrium (LD) or the degree of correlation between an allele at a PS in any of Tables A-1 through A-20 (Appendix A) and a candidate substituting allele at a polymorphic site located elsewhere in the relevant gene or on the relevant chromosome. Similarly, alternate genetic markers comprising a linked polymorphism are readily identified by determining the degree of LD between a marker in any of Tables A-1 through A-20 (Appendix A) and a candidate linked polymorphism located elsewhere in the relevant gene or on the relevant chromosome. The candidate substituting allele or linked polymorphism may be a polymorphism that is currently known. Other candidate substituting alleles and linked polymorphisms may be readily identified by the skilled artisan using any technique well-known in the art for discovering polymorphisms.

The degree of LD between a genetic marker in any of Tables A-1 through A-20 (Appendix A) and a candidate alternate polymorphism may be determined using any LD measurement known in the art. LD patterns in genomic regions are readily determined empirically in appropriately chosen samples using various techniques known in the art for determining whether any two alleles (e.g., between SNPs at different PSs or between two haplotypes) are in linkage disequilibrium. (GENETIC DATA ANALYSIS II, Weir, Sinauer Associates, Inc., Sunderland, Mass. (1996)). The skilled artisan may readily select which method of determining LD will be best suited for a particular sample size and genomic region.

One of the most frequently used measures of linkage disequilibrium is Δ², which is calculated using the formula described by Devlin et al. (Genomics 29(2):311-22 (1995)). Δ² is the measure of how well an allele X at a first locus predicts the occurrence of an allele Y at a second locus on the same chromosome. The measure only reaches 1.0 when the prediction is perfect (e.g., X if and only if Y).

In preferred alternate genetic markers, the locus of a substituting allele or a linked polymorphism is in a genomic region of about 100 kilobases spanning the relevant gene, and more preferably, the locus is in the relevant gene. Other preferred alternate genetic markers are those in which the LD or correlation between the relevant alleles (e.g., between the substituting SNP and the substituted SNP, or between the linked polymorphism and the haplotype) has a Δ² or r² (the square of correlation coefficient) value, as measured in a suitable reference population, of at least 0.75, more preferably at least 0.80, even more preferably at least 0.85 or at least 0.90, yet more preferably at least 0.95, and most preferably 1.0. The reference population used for this Δ² or r² measurement preferably reflects the genetic diversity of the population of patients that are candidates for treatment with antidepressants. For example, the reference population may be the general population, a population using the drug, a population diagnosed with a particular condition for which the drug shows efficacy, or a population of similar ethnic background.

Individuals having any of the genetic markers described herein are likely to respond to antidepressants. Preferred genetic markers of the invention comprise any of the markers in Tables A-1 through A-20 (Appendix A).

### III. Detecting Markers of Antidepressant Response

In all of the embodiments of the invention, the skilled artisan will appreciate that detecting the presence or absence of a specific genetic marker in a marker group in an individual is also literally equivalent to detecting the presence or absence of the same copy number of a substitute, linked or correlated polymorphism for the polymorphism in that specific marker in which Δ²=1 for the linkage disequilibrium or the correlation coefficient=1 between the substituted polymorphism in that marker and the substituting polymorphism.

The presence in an individual of a genetic marker of the invention may be determined by any of a variety of methods well known in the art that permits the determination of whether the individual has the required copy number of the polymorphism comprising the marker. For example, if the required copy number is one or two, then the method need only determine that the individual has at least one copy of the polymorphism. In preferred embodiments, the method provides a determination of the actual copy number.

Typically, these methods involve assaying a nucleic acid sample prepared from a biological sample obtained from the individual to determine the identity of a nucleotide or nucleotide pair present at one or more polymorphic sites in the marker. Nucleic acid samples may be prepared from virtually any biological sample. For example, convenient samples include whole blood, serum, semen, saliva, tears, fecal matter, urine, sweat, buccal matter, skin and hair. Preferred samples contain only somatic cells, and such samples would typically be required when the locus is on an autosomal or X chromosome. Nucleic acid samples may be prepared for analysis using any technique known to those skilled in the art. Preferably, such techniques result in the production of genomic DNA sufficiently pure for determining the genotype or haplotype pair for a desired set of polymorphic sites in the nucleic acid molecule. Such techniques may be found, for example, in MOLECULAR CLONING: A LABORATORY MANUAL, Sambrook et al., Cold Spring Harbor Laboratory, New York (2001), which is incorporated herein by reference.

For markers in which the specified polymorphism is a haplotype, the copy number of the haplotype in the nucleic acid sample may be determined by a direct haplotyping method or by an indirect haplotyping method, in which the haplotype pair for the set of polymorphic sites comprising the marker is inferred from the individual's haplotype genotype for that set of PSs. The way the nucleic acid sample is prepared depends on whether a direct or indirect haplotyping method is used.

Direct haplotyping methods typically involve treating a genomic DNA sample isolated from a blood or cheek sample obtained from the individual in a manner that produces a hemizygous DNA sample that contains only one of the individual's two alleles for the locus which, as readily understood by the skilled artisan, may be the same allele or different alleles, and detecting the nucleotide present at each PS of interest. The nucleic acid sample may be obtained using a variety of methods known in the art for preparing hemizygous DNA samples, which include: targeted in vivo cloning (TIVC) in yeast as described in WO 98/01573, U.S. Pat. Nos. 5,866,404 and 5,972,614; generating hemizygous DNA targets using an allele specific oligonucleotide in combination with primer extension and exonuclease degradation as described in U.S. Pat. No. 5,972,614; single molecule dilution (SMD) as described in Ruaño et al., Proc. Natl. Acad. Sci. U.S.A. 87:6296-300 (1990); and allele specific PCR (Ruaño et al., Nucl. Acids Res. 17:8392 (1989); Ruaño et al., Nucl. Acids Res. 19:6877-82 (1991); Michalatos-Beloin et al., infra (1996)).

As will be readily appreciated by those skilled in the art, if the individual is expected to have two alleles for the locus (e.g., the locus is on an autosomal chromosome, or the locus is on the X chromosome and the individual is a female), any individual clone of the locus in that individual will permit directly determining the haplotype for only one of the two alleles; thus, additional clones will need to be examined to directly determine the identity of the haplotype for the other allele. Typically, at least five clones of the genomic locus present in the individual should be examined to have more than a 90% probability of determining both alleles. In some cases, however, once the haplotype for one allele is directly determined, the haplotype for the other allele may be inferred if the individual has a known genotype for the PSs comprising the marker or if the frequency of haplotypes or haplotype pairs for the locus in an appropriate reference population is available.

Direct haplotyping of both alleles may be performed by assaying two hemizygous DNA samples, one for each allele, that are placed in separate containers. Alternatively, the two hemizygous samples may be assayed in the same container if the two samples are labeled with different tags, or if the assay results for each sample are otherwise separately distinguishable or identifiable. For example, if the samples are labeled with first and second fluorescent dyes, and a PS in the locus is assayed using an oligonucleotide probe that is specific for one of the alleles—and labeled with a third fluorescent dye, then detecting a combination of the first and third dyes would identify the nucleotide present at the PS in the first sample while detecting a combination of the second and third dyes would identify the nucleotide present at the PS in the second sample.

Indirect haplotyping methods typically involve preparing a genomic DNA sample isolated from a blood or cheek sample obtained from the individual in a manner that permits accurately determining the individual's genotype for each PS in the locus. The genotype is then used to infer the identity of at least one of the individual's haplotypes for the locus, and preferably used to infer the identity of the individual's haplotype pair for the locus.

In one indirect haplotyping method, the presence of zero, one or two copies of a haplotype of interest can be determined by comparing the individual's genotype for the PS in the marker with a set of reference haplotype pairs for the same set of PS and assigning to the individual a reference haplotype pair that is most likely to exist in the individual. The individual's copy number for the haplotype comprising the marker is how many copies of that haplotype is in the assigned reference haplotype pair.

The reference haplotype pairs are those that are known to exist in the general population or in a reference population, or that are theoretically possible based on the alternative alleles possible at each PS. The reference population may be composed of randomly-selected individuals representing the major ethnogeographic groups of the world. A preferred reference population is one having a similar ethnogeographic background as the individual being tested for the presence of the marker. The size of the reference population is chosen based on how rare a haplotype is that one wants to be guaranteed to see. For example, if one wants to have a q% chance of not missing a haplotype that exists in the population at a p% frequency of occurring in the reference population, the number of individuals (n) who must be sampled is given by 2n=log(1−q)/log(1−p) where p and q are expressed as fractions. A particularly preferred reference population includes one or more 3-generation families to serve as a control for checking quality of haplotyping procedures. If the reference population comprises more than one ethnogeographic group, the frequency data for each group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3^{rd} Ed., 1997) postulates that the frequency of finding the haplotype pair H₁/H₂ is equal to p_{H-W}(H₁/H₂)=2p(H₁)p(H₂) if H₁≠H₂ and p_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, the method disclosed in U.S. Pat. No. 5,866,404, single molecule dilution, or allele-specific long-range PCR. (Michalotos-Beloin et al., Nucleic Acids Res. 24:4841-4843 (1996)).

Assignment of the haplotype pair may be performed by choosing a reference haplotype pair that is consistent with the individual's genotype. When the genotype of the individual is consistent with more than one reference haplotype pair, the frequencies of the reference haplotype pairs may be used to determine which of these consistent haplotype pairs is most likely to be present in the individual. If a particular consistent haplotype pair is more frequent in the reference population than other consistent haplotype pairs, then the consistent haplotype pair with the highest frequency is the most likely to be present in the individual. Occasionally, only one haplotype represented in the reference haplotype pairs is consistent with any of the possible haplotype pairs that could explain the individual's genotype, and in such cases the individual is assigned a haplotype pair containing this known haplotype and a new haplotype derived by subtracting the known haplotype from the possible haplotype pair. In rare cases, either no haplotypes in the reference population are consistent with the individual's genotype, or alternatively, multiple reference haplotype pairs are consistent with the genotype. In such cases, the individual is preferably haplotyped using a direct molecular haplotyping method.

Indirect determination of the copy number of haplotypes present in an individual from her genotype is illustrated here for a hypothetical Marker X, which is associated with antidepressant response. Marker X consists of one or two copies of Haplotype GA, which contains two polymorphic sites, PSA and PSB, in Gene Y on an autosomal chromosome. The hypothetical below shows the 9 (3ⁿ, where each of n=2 bi-allelic polymorphic sites may have one of 3 different genotypes present) genotypes that may be detected for the set of PSA and PSB, using a genomic DNA sample from an individual. Eight of the nine possible genotypes for the two sites allow unambiguous determination of the number of copies of Haplotype GA present in the individual and therefore would allow unambiguous determination of the presence or absence in the individual of Marker X. However, an individual with the C/G A/C genotype could possess either of the following haplotype pairs: CA/GC or CC/GA, and thus could have either 1 copy of Haplotype GA (CC/GA haplotype pair), which would mean Marker X is present, or 0 copy (CA/GC haplotype pair) of Haplotype GA, which would mean Marker X is absent. For this instance where there is ambiguity in the haplotype pair underlying the determined genotype C/G A/C, frequency information may be used to determine the most probable haplotype pair and therefore the most likely number of copies of the marker haplotype in the individual, as described above. Alternatively, for the ambiguous double heterozygote, genotyping of one or more additional sites in Gene Y or nearby may be performed to resolve this ambiguity. The skilled artisan would recognize that these one or more additional sites would need to have sufficient linkage with the alleles in at least one of the haplotypes in a possible haplotype pair to permit unambiguous assignment of that haplotype pair. Although this illustration has been directed to the particular instance of determining the number of Haplotype AG present in an individual, an analogous process would be used for determining the copy number of any linked or substitute haplotype for Haplotype AG.

Hypothetical: Possible copy numbers of Haplotype (GA)
Derived From Possible Genotypes at PSA and PSB

| | |
|---|---|
| Genotype | Copy Number of |

| PSA | PSB | Haplotype GA |
|---|---|---|
| G/G | C/C | 0 |
| G/G | A/C | 1 |
| C/G | C/C | 0 |
| C/G | A/C | 1 or 0 |
| G/C | A/A | 1 |
| G/G | A/A | 2 |
| C/C | A/A | 0 |
| C/C | A/C | 0 |
| C/C | C/C | 0 |

Any of all of the steps in the indirect haplotyping method described above may be performed manually, by visual inspection and performing appropriate calculations, but are preferably performed by a computer-implemented algorithm that accesses data on the individual's genotype and reference haplotype pairs stored in computer readable format. Such algorithms are described in WO 01/80156 and WO 05/048012. Alternatively, the haplotype pair in an individual may be predicted from the individual's genotype for that gene with the assistance of other reported haplotyping algorithms (See, e.g., Clark et al., Mol. Bio. Evol. 7:111-22 (1990); Stephens et al., Am. J. Hum. Genet. 68:978-89 (2001); WO 02/064617; Niu et al., Am. J. Hum. Genet. 70:157-69 (2002); Zhang et al., BMC Bioinformatics 4(1):3 (2003)) or through a commercial haplotyping service.

All direct and indirect haplotyping methods described herein typically involve determining the identity of at least one of the alleles at a PS in a nucleic acid sample obtained from the individual. To enhance the sensitivity and specificity of that determination, it is frequently desirable to amplify from the nucleic acid sample one or more target regions in the locus. An amplified target region may span the locus of interest, such as an entire gene, or a region thereof containing one or more polymorphic sites. Separate target regions may be amplified for each PS in a marker.

Any amplification technique known to those of skill in the art may be used in practicing the present invention including, but not limited to, polymerase chain reaction (PCR) techniques. PCR may be carried out using materials and methods known to those of skill in the art. (See generally PCR TECHNOLOGY: PRINCIPALS AND APPLICATIONS FOR DNA AMPLIFICATION, ed. Erlich, Freeman Press, New York (1992); PCR PROTOCOLS: A GUIDE TO METHODS AND APPLICATIONS, eds. Innis et al., Academic Press, San Diego (1990); Matilla et al., Nucleic Acids Res. 19: 4967 (1991); Eckert et al., PCR Methods and Applications 1:17 (1991); PCR 2: A PRACTICAL APPROACH, eds. McPherson et al., IRL Press, Oxford (2000); and U.S. Pat. No. 4,683,202). Other suitable amplification methods include the ligase chain reaction (LCR) (see Wu et al., Genomics 4:560 (1989) and Landegren et al., Science 241:1077 (1988)), transcription amplification (Kwoh et al., Proc. Natl. Acad. Sci. U.S.A. 86:1173 (1989)), self-sustained sequence replication (Guatelli et al., Proc. Nat. Acad. Sci. U.S.A. 87:1874 (1990)); isothermal methods (Walker et al., Proc. Natl. Acad. Sci. U.S.A. 89:392-6 (1992)); and nucleic acid-based sequence amplification (NASBA).

The amplified target region is assayed to determine the identity of at least one of the alleles present at a PS in the region. If both alleles of a locus are represented in the amplified target, it will be readily appreciated by the skilled artisan that only one allele will be detected at a PS in individuals who are homozygous at that PS, while two different alleles will be detected if the individual is heterozygous for that PS. The identity of the allele may be identified directly, known as positive-type identification, or by inference, referred to as negative-type identification. For example, where a SNP is known to be guanine or cytosine in a reference population, a PS may be positively determined to be either guanine or cytosine for an individual homozygous at that site, or both guanine and cytosine, if the individual is heterozygous at that site. Alternatively, the PS may be negatively determined to be not guanine (and thus cytosine/cytosine) or not cytosine (and thus guanine/guanine).

Identifying the allele or pair of alleles at a PS may be accomplished using any technique known to those of skill in the art. Preferred techniques permit rapid, accurate assaying of multiple PS with a minimum of sample handling. Some examples of suitable techniques include, but are not limited to, direct DNA sequencing of the amplified target region, capillary electrophoresis, hybridization of allele-specific probes, single-strand conformation polymorphism analysis, denaturing gradient gel electrophoresis, temperature gradient electrophoresis, mismatch detection; nucleic acid arrays, primer specific extension, protein detection, and other techniques well known in the art. (See, e.g., MOLECULAR CLONING: A LABORATORY MANUAL, Sambrook et al., Cold Spring Harbor Laboratory, New York (2001); CURRENT PROTOCOLS IN MOLECULAR BIOLOGY, Ausubel et al., John Wiley and Sons, New York (1997); Orita et al., Proc. Nat. Acad. Sci. U.S.A. 86:2766-70 (1989); Humphries et al. in MOLECULAR DIAGNOSIS OF GENETIC DISEASES, ed. Elles, pp. 321-30, Humana Press, Totowa, N.J. (1996); Wartell et al., Nucl. Acids Res. 18:2699-706 (1990); Hsu et al., Carcinogenesis 15:1657-62 (1994); Sheffield et al., Proc. Natl. Acad. Sci. U.S.A. 86:232-6 (1989); Winter et al., Proc. Natl. Acad. Sci. U.S.A. 82:7575 (1985); Myers et al., Nature 313:495 (1985); Rosenbaum et al., Biophys. Chem. 265:12753 (1987); Modrich, Ann. Rev. Genet. 25:229-53 (1991); U.S. Pat. Nos. 6,300,063, 5,837,832, and 5,459,039; and HuSNP Mapping Assay, reagent kit and user manual, Affymetrix Part No. 90094 (Affymetrix, Santa Clara, Calif.)).

In preferred embodiments, the identity of allele(s) at a PS is determined using a polymerase-mediated primer extension method. Several such methods have been described in the patent and scientific literature and include the “Genetic Bit Analysis” method (WO 92/15712) and the ligase/polymerase mediated genetic bit analysis. (U.S. Pat. No. 5,679,524). Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798. Another primer extension method is allele-specific PCR. (Ruaño et al., supra (1989); Ruaño et al., supra (1991); WO 93/22456; Turki et al., J. Clin. Invest. 95:1635-41 (1995)). In addition, multiple PSs may be investigated by simultaneously amplifying multiple regions of the nucleic acid using sets of allele-specific primers as described in WO 89/10414.

Another primer extension method for identifying and analyzing polymorphisms employs single-base extension (SBE) of a fluorescently-labeled primer coupled with fluorescence resonance energy transfer (FRET) between the label of the added base and the label of the primer. Typically, the method, such as that described by Chen et al. (Proc. Nat. Acad. Sci. U.S.A. 94:10756-61 (1997)), uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

In all of the above methods, the accuracy and specificity of an assay designed to detect the identity of the allele(s) at any PS is typically validated by performing the assay on DNA samples in which the identity of the allele(s) at that PS is known. Preferably a sample representing each possible allele is included in the validation process. For diploid loci such as those on autosomal and X chromosomes, the validation samples will typically include a sample that is homozygous for the major allele at the PS, a sample that is homozygous for the minor allele at the PS, and a sample that is heterozygous at that PS. These validation samples are typically also included as controls when performing the assay on a test sample (i.e., a sample in which the identity of the allele(s) at the PS is unknown). The specificity of an assay may also be confirmed by comparing the assay result for a test sample with the result obtained for the same sample using a different type of assay, such as by determining the sequence of an amplified target region believed to contain the PS of interest and comparing the determined sequence to a context sequence based on the reference sequence of the relevant gene. The length of the context sequence necessary to establish that the correct genomic position is being assayed will vary based on the uniqueness of the sequence in the target region (for example, there may be one or more highly homologous sequences located in other genomic regions). The skilled artisan can readily determine an appropriate length for a context sequence for any PS using known techniques such as blasting the context sequence against publicly available sequence databases. For amplified target regions, which provide a first level of specificity, examining the context sequence of about 30 to 60 bases on each side of the PS in known samples is typically sufficient to ensure that the assay design is specific for the PS of interest. Occasionally, a validated assay may fail to provide an unambiguous result for a test sample. This is usually the result of the sample having DNA of insufficient purity or quantity, and an unambiguous result is usually obtained by repurifying or reisolating the DNA sample or by assaying the sample using a different type of assay.

Alternatively, the presence or absence of a marker of the invention may be detected by detecting, in a protein sample obtained from the individual, a polypeptide specified by the polymorphism comprising the marker. The polypeptide may be detected using a monoclonal antibody specific for that polypeptide.

Further, in performing any of the methods described herein that require determining the presence or absence of a marker of antidepressant response, such determination may be made by consulting a data repository that contains sufficient information on the patient's genetic composition to determine whether the patient has the marker. Preferably, the data repository lists what marker(s) are present and absent in the individual. The data repository could include the individual's patient records, a medical data card, a file (e.g., a flat ASCII file) accessible by a computer or other electronic or non-electronic media on which appropriate information or genetic data can be stored. As used herein, a medical data card is a portable storage device such as a magnetic data card, a smart card, or a flash-memory card. If the data repository is a file accessible by a computer; such files may be located on various media, including: a server, a client, a hard disk, a CD, a DVD, a personal digital assistant, a tape, a zip disk, the computer's internal ROM (read-only-memory) or the internet or worldwide web. Other media for the storage of files accessible by a computer will be obvious to one skilled in the art.

### IV. Utility of Markers of Antidepressant Response

The phenotypic effect of the markers described herein support using these markers in a variety of methods and products, including, but not limited to, diagnostic methods and kits.

The utility of any of the methods or products described herein is not dependent on complete correlation between the presence of a genetic marker of the invention and the occurrence of antidepressant response, or upon whether a diagnostic or treatment method or kit is 100% accurate, or has an specific degree of accuracy, in determining the presence or absence of a genetic marker in every individual, or in predicting for every individual whether the individual will respond to an antidepressant. Thus, the inventors herein intend that the terms “determine,” “determining,” and “predicting” should not be interpreted as requiring a definite or certain result; instead these terms should be construed as meaning that a claimed method or kit provides an accurate result for the majority of individuals, or that the result or prediction for any given individual is more likely to be correct than incorrect. Preferably, the accuracy of the result provided by a diagnostic method or kit of the invention is one that a skilled artisan or regulatory authority would consider suitable for the particular application in which the method or kit is used.

An individual to be tested in any of the methods described herein is a human subject that is a candidate for treatment with antidepressants. In some embodiments, the individual has been diagnosed with, or exhibits a symptom of, a disease for which an antidepressant is approved. In other embodiments, the antidepressant is not approved for treating the diagnosed disease or exhibited symptom(s), but the prescribing physician believes the drug may be helpful in treating the individual. In preferred embodiments of the invention, the individual has any disease or condition for which the antidepressant has displayed some degree of clinical utility.

A. Diagnostic Methods and Kits

The diagnostic methods and kits of the invention are useful in clinical diagnostic applications. However, as used herein, the term “diagnostic” is not limited to clinical or medical uses, and that diagnostic methods and kits of the invention claimed herein are also useful in any research application in which it is desirable to test a subject for the presence or absence of any genetic marker described in Section II above. In preferred embodiments, the diagnostic methods and kits of the invention test for, or are designed to test for, respectively, the presence or absence of a set of ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD markers, which set may comprise a marker from, respectively, Tables A-1 through A-20 (Appendix A), or may comprise all ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD markers described herein.

It is contemplated that any or all of the diagnostic methods claimed herein may be performed by a testing laboratory on an individual's biological sample provided directly by the individual or by any third party, such as the individual's physician, a relative of the individual, a person conducting a research study in which the individual is participating and the like. The third party may have a commercial relationship with the testing laboratory, or may be totally independent thereof. Where the results of the diagnostic method is to be used for clinical purposes, the testing laboratory is preferably a clinical laboratory who performs the diagnostic method in compliance with all applicable laws and regulations in the locality where the testing is performed as well as where the individual resides.

In some embodiments, the testing laboratory does not know the identity of the individual whose sample it is testing. For example, the sample may be merely identified by a number or some other code (a “sample ID”) and the results of the diagnostic method can be reported to the party ordering the test using the sample ID. In preferred embodiments, the link between the identity of an individual and the individual's sample is known only to the individual or to the individual's physician. In other applications, such as research studies, the link may be broken prior to the testing laboratory sending a report of the results; thus, the results cannot be obtained by the individual or the individual's insurance company.

Kits of the invention, which are useful for detecting the presence or absence of a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker in an individual, comprise a set of oligonucleotides designed for identifying each of the alleles at each PS in the marker. In preferred embodiments, the set of oligonucleotides is designed to identify the alleles at all polymorphic sites in all of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD markers described herein. In particularly preferred embodiments, the set of oligonucleotides is designed to identify both alleles at each PS in a set of ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD markers, with the marker set comprising a marker from, respectively, Tables A-1 through A-20 (Appendix A).

In some embodiments, the oligonucleotides in the kit are either allele-specific probes or allele-specific primers. In other embodiments, the kit comprises primer-extension oligonucleotides. In still further embodiments, the set of oligonucleotides is a combination of allele-specific probes, allele-specific primers, or primer-extension oligonucleotides. The kit may comprise oligonucleotides designed for genotyping other PS, which may be in the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD gene or at any other locus of interest in the human genome.

Oligonucleotides in kits of the invention must be capable of specifically hybridizing to a target region of a polynucleotide. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. In some embodiments, the target region contains a PS in a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker, while in other embodiments, the target region is located one to 10 nucleotides from the PS.

The composition and length of each oligonucleotide in the kit will depend on the nature of the genomic region containing the PS as well as the type of assay to be performed with the oligonucleotide and is readily determined by the skilled artisan. For example, the polynucleotide to be used in the assay may constitute an amplification product, and thus the required specificity of the oligonucleotide is with respect to hybridization to the target region in the amplification product rather than in genomic DNA isolated from the individual. As another example, if each PS in a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker is to be assayed simultaneously, the melting temperatures for the oligonucleotides in the kit will typically be within a narrow range, preferably less than about 5° C. and more preferably less than about 2° C.

In preferred embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting polymorphisms, departures from complete complementarity are contemplated where such departures do not prevent the molecule from specifically hybridizing to the target region as defined above. For example, an oligonucleotide primer may have a non-complementary fragment at its 5′ end, with the remainder of the primer being completely complementary to the target region. Alternatively, non-complementary nucleotides may be interspersed into the probe or primer as long as the resulting probe or primer is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. As the target sequences are generally present in excess, at Tₘ, 50% of the probes are occupied at equilibrium. Typically, stringent conditions include a salt concentration of at least about 0.01 to 1.0 M Na ion concentration (or other salts) at pH 7.0 to 8.3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations. Additional stringent conditions can be found in chapters 7, 9, and 11 in MOLECULAR CLONING: A LABORATORY MANUAL, Sambrook et al., Cold Spring Harbor Laboratory, New York (2001), and in NUCLEIC ACID HYBRIDIZATION, A PRACTICAL APPROACH, Haymes et al., IRL Press, Washington, D.C. (1985).

A preferred, non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41 (%G+C)−(600/N), where N is the number of bases in the hybrid, and [Na⁺] is the concentration of sodium ions in the hybridization buffer ([Na⁺] for 1×SSC=0.165 M).

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide (peptide nucleic acid (PNA)) and the like. (See Varma in MOLECULAR BIOLOGY AND BIOTECHNOLOGY: A COMPREHENSIVE DESK REFERENCE, ed. Meyers, pp. 617-20, VCH Publishers, Inc. (1995)). The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may be constitute components of an approved diagnostic device.

In some embodiments, the set of oligonucleotides in the kit have different labels to allow determining the identity of the alleles at two or more PSs simultaneously. The oligonucleotides may also comprise an ordered array that is immobilized on a solid surface such as a microchip, bead, or glass slide (see, e.g., WO 98/20020 and WO 98/20019). Kits comprising such immobilized oligonucleotides may be designed to perform a variety of polymorphism detection assays, including but not limited to probe hybridization and polymerase extension assays.

Kits of the invention may also contain other reagents such as hybridization buffer (e.g., where the oligonucleotides are to be used as allele-specific probes) or dideoxynucleotide triphosphates (ddNTPs; e.g., where the alleles at the polymorphic sites are to be detected by primer extension). Kits designed for use in polymerase-mediated genotyping assays, may also contain a polymerase and a reaction buffer optimized for the polymerase-mediated assay to be performed. Kits of the invention may also include reagents to detect when a specific hybridization has occurred or a specific polymerase-mediated extension has occurred. Such detection reagents may include biotin- or fluorescent-tagged oligonucleotides or ddNTPs and/or an enzyme-labeled antibody and one or more substrates that generate a detectable signal when acted on by the enzyme. It will be understood by the skilled artisan that the set of oligonucleotides and reagents for performing the assay will be provided in separate receptacles placed in the kit container if appropriate to preserve biological or chemical activity and enable proper use in the assay.

In other preferred embodiments, each of the oligonucleotides and all other reagents in the kit have been quality tested for optimal performance in an assay designed to determine each of the alleles at the set of PSs comprising a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker. In more preferred embodiments, the kit includes an instruction manual that describes the various ways the kit may be used to detect the presence or absence of a ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker.

In some preferred embodiments, the set of oligonucleotides in the kit are allele-specific oligonucleotides. As used herein, the term allele-specific oligonucleotide (ASO) means an oligonucleotide that is able, under sufficiently stringent conditions, to hybridize specifically to one allele of a PS, at a target region containing the PS while not hybridizing to the same region containing a different allele. As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes and primers are found in Kogan et al. in PCR PROTOCOLS: A GUIDE TO METHODS AND APPLICATIONS, eds. Innis et al., Academic Press, San Diego (1990), and Ruaño et al., supra (1990).

Typically, an ASO will be perfectly complementary to one allele while containing a single mismatch for another allele. In ASO probes, the single mismatch is preferably within a central position of the oligonucleotide probe as it aligns with the polymorphic site in the target region (e.g., approximately the 7^{th} or 8^{th} position in a 15mer, the 8^{th} or 9^{th} position in a 16mer, and the 10^{th} or 11^{th} position in a 20mer). The single mismatch in ASO primers is located at the 3′ terminal nucleotide, or preferably at the 3′ penultimate nucleotide. ASO probes and primers hybridizing to either the coding or noncoding strand are contemplated by the invention.

In other preferred embodiments, the kit comprises a pair of allele-specific oligonucleotides for each PS to be assayed, with one member of the pair being specific for one allele and the other member being specific for the other allele. In such embodiments, the oligonucleotides in the pair may have different lengths or have different detectable labels to allow the user of the kit to determine which allele-specific oligonucleotide has specifically hybridized to the target region, and thus determine which allele is present in the individual at the assayed PS.

In still other preferred embodiments, the oligonucleotides in the kit are primer-extension oligonucleotides. Termination mixes for polymerase-mediated extension from any of these oligonucleotides are chosen to terminate extension of the oligonucleotide at the PS of interest, or one base thereafter, depending on the alternative nucleotides present at the PS.

B. Methods of Treatment

In addition to diagnostic methods and kits, the invention provides a method for treating depression in an individual. This method comprises determining the presence or absence in the individual of an ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, or NCALD marker, and making a treatment decision based on the results. If it is determined that the marker is present, then the decision is to prescribe to the individual the lowest approved dose of an antidepressant. If however it is determined that the marker is absent, then the decision is to either prescribe to the individual the antidepressant at a dose that is higher than the lowest approved dose, or prescribe to the individual a therapy not including the antidepressant that is effective in treating depression.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### EXAMPLES

The Examples herein are meant to exemplify the various aspects of carrying out the invention and are not intended to limit the scope of the invention in any way. The Examples do not include detailed descriptions for conventional methods employed, such as the design of PCR primers, performing PCR, and haplotyping. Such methods are well known to those skilled in the art and are described herein or in numerous publications, for example, MOLECULAR CLONING: A LABORATORY MANUAL, Sambrook et al., Cold Spring Harbor Laboratory, New York (2001).

### Example 1

Vilazodone is an indolealkylamine derivative under development for the treatment of MDD. The full chemical designation is 5-{4-[4-(5-Cyano-3-indolyl)-butyl]-1-piperazinyl}-benzofuran-2-carboxamide hydrochloride. Vilazodone has two major components to its pharmacological profile: it is a potent and selective serotonin reuptake inhibitor (SSRI), and it is a partial 5-HT1A receptor agonist. It is anticipated that this dual mechanism of action will confer additional benefits for both efficacy and some aspects of tolerability relative to pure SSRIs.

The importance of the 5-HT1A receptor in the response to antidepressant drugs has attracted much attention in the scientific literature. Artigas et al. have reported improved response to treatment and improved response time after combination therapy with the 5-HT1A antagonist/partial agonist pindolol added to SSRIs. (Arch. Gen. Psychiatry 54:248 (1991)). The mechanism underlying this augmentation effect is not yet clear in view of the complex actions of these drugs at pre-, post-, and somato-dendritic receptor sites. These types of studies suggest that, through a dual or multiple target approach, it should be possible to develop new antidepressants that will improve the treatment of depression. Drugs such as vilazodone, which combines serotonin reuptake blockade and agonism of the 5-HT1A receptor, are therefore promising opportunities in depression treatment.

As part of the vilazodone development program, five Phase II studies have been conducted, all in patients with MDD. These studies randomized 2,098 patients for eight weeks of treatment, 1196 of whom received vilazodone. The most common adverse events, reported by at least ten percent of vilazodone-treated patients are nausea, diarrhea, headache, insomnia, dizziness, dry mouth, somnolence, abnormal dreams, and vomiting. A pre-clinical study in beagle dogs showed treatment emergent corneal opacities and reduced tear production. Hence, thorough opthalmologic examinations have also been conducted in these studies. The only clinically significant finding from these examinations was an increase in the incidence of mild ocular drying in subjects treated with vilazodone.

Although vilazodone has been generally shown to be safe, efficacy has not yet been established. In each of the Phase II studies, vilazodone did not show a statistically significant difference on the primary efficacy variable compared to placebo. It should be noted, however, that in the three studies in which an active comparator already approved for the treatment of MDD was used (citalopram or fluoxetine), the active comparator also did not show a statistically significant improvement in Hamilton Depression Scale (HAM-D) total score versus placebo. Overall, the placebo response rate in these studies was 48% when defined as a 50% reduction from baseline in HAM-D, thereby limiting the ability of these studies to demonstrate the efficacy of vilazodone or of the active comparators.

Exploratory analyses, using established methods for enriching patient groups to differentiate between active treatment and placebo, demonstrate the potential for demonstrating the efficacy of vilazodone. For example, by evaluating response stratified by baseline disease severity, a larger treatment effect is observed for more severely depressed patients.

There was a wide range of inter-individual variability in response to vilazodone as well as to placebo and the active comparators. This variability may be partially due to genetic differences among the individuals treated. This example illustrates a randomized, double-blind, placebo-controlled, multicenter, eight-week study designed to discover genetic markers of response to vilazodone in adult patients diagnosed with MDD by the DSM-IV-TR criteria. Patients must have met all of the following inclusion criteria to be considered for enrollment in the study: (1) 18-65 years of age; (2) diagnosis of MDD, single episode or recurrent, according to DSM-IV-TR (296.2/296.3) with a current Major Depressive Episode of less than one year's duration with a minimum duration of at least 4 weeks; (3) HAM-D score≧22 on the first 17 items of the 21-item HAM-D at screening and baseline visits; (4) HAM-D item 1 (depressed mood) score≧2 at the screening visit and the baseline visit; (5) provision of written informed consent to participate; and (6) ability to speak, read and understand English and to respond to questions and follow simple instructions.

Patients meeting any of the following exclusion criteria were excluded from the study: (1) current (or within six months prior to the screening visit) Axis I disorder of Post Traumatic Stress Disorder, Eating Disorder, or Obsessive Compulsive Disorder (Generalized Anxiety Disorder, Social Phobia or Simple Phobia were allowed); (2) history of schizophrenia, schizoaffective disorder or bipolar I or II disorder (with a history of hypomanic or manic episodes); (3) substance abuse (alcohol or drugs) (DSM-IV-TR) within three months prior to the screening visit or substance dependence within six months prior to the screening visit, (4) possession of any of the following DSM-IV-TR MDD Specifiers: (a) Catatonic Features, (b) Postpartum Onset, and (c) Seasonal Pattern; (5) receipt of psychotherapy within the twelve weeks prior to the screening visit; (6) patients who, in the investigator's judgment, posed a serious suicidal or homicidal risk at the screening visit or the baseline visit or who made a suicide attempt within six months prior to the screening visit; (7) patients who had an inadequate response to at least two consecutive antidepressants from different classes given at adequate doses for an adequate duration; (8) patients who received electroconvulsive therapy within the six months prior to the screening visit; (9) patients who had taken psychotropic drugs (patients taking psychotropic drugs must have discontinued these prior to the screening visit, with the minimum discontinuation periods being four weeks prior to the screening visit for monoamine oxidase inhibitors (MAOIs) and fluoxetine, two weeks prior to the screening visit for all other antidepressants, sedatives, hypnotics, beta adrenergic blockers, benzodiazepines or other psychoactive medications (including psychoactive herbal treatments); and twelve weeks prior to the screening visit for depot neuroleptics; (10) patients taking migraine medications with a serotonergic mechanism of action (e.g., sumatriptan, naritriptan, ergot derivatives); (11) patients with a known hypersensitivity to SSRIs or 5-HT1a agonists; (12) patients previously treated with vilazodone; (13) patients with a history of clinically significant cardiac, renal, neurologic, cerebrovascular, hepatic, hematologic, metabolic or pulmonary disease, including patients who had a myocardial infarction within one year prior to the screening visit, patients with diabetes mellitus who require insulin treatment (oral antidiabetic agents allowed), patients with a history of seizure disorders (except for febrile seizures in childhood), patients with a prior or current history of neoplastic disease (squamous cell carcinoma of the skin allowed), patients with renal impairment (serum creatinine≧1.7 mg/dL) or hepatic impairment (ALT or AST three times the upper limit of normal), patients who are not euthyroid, unless on thyroid medication (patients maintained on thyroid medication must have been euthyroid for a period of at least six months prior to the screening visit); patients with any serious medical disorder or condition that would have, in the investigator's opinion, precluded the administration of study medication; (14) patients with Sicca syndrome; (15) pregnant or lactating patients, or patients planning on becoming pregnant during the study [all female patients who were not at least one year post menopausal or irreversibly surgically sterilized must have had a negative urine pregnancy test at Visit 1 and 2, and must have been determined to not be at risk of pregnancy and/or must have been using adequate and reliable contraception throughout the trial, with adequate contraception being defined as continuous use of any of Norplant® (inserted at least 3 months prior to administration); medroxyprogesterone acetate injection (given >14 days prior to study entry); oral contraception (taken as directed for >1 month prior to study entry); double-barrier method (e.g., condom and spermicide); intrauterine device (inserted >4 weeks prior to study entry); or monogamous partner with a bilateral vasectomy (procedure performed >3 months prior to study entry)]; (16) patients with clinically significant abnormalities on ECG (abnormalities determined by the investigator and the physician interpreting the ECG) not resolved at the baseline visit; (17) patients having clinically significant abnormal laboratory findings at the screening visit not resolved at the baseline visit; (18) patients with a positive drug screen (one re-screen after four weeks was allowed for those patients testing positive for marijuana); (19) patients who, in the opinion of the investigator, would have been noncompliant with the visit schedule or study procedures (e.g., illiteracy, planned vacations or planned hospitalizations during the study); and (20) patients that had taken an investigational drug or participated in an investigational drug trial within 30 days prior to the screening visit.

Following randomization into vilazodone and placebo groups, patients began the forced titration schedule. Patients received 10 mg qd until Visit 3 (approximately Day 7). At Visit 3 patients received 20 mg qd until Visit 4 (approximately Day 14). At Visit 4 patients received the target dose of 40 mg qd (approximately Day 15). A total of 152 patients completed the vilazodone arm, while 154 patients completed the placebo arm.

### Example 2

This example illustrates genotyping of the study group for the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, and PSMD1 polymorphic sites selected by the inventors herein for analysis. Genomic DNA was isolated from blood obtained from each individual and amplified target regions containing the polymorphic sites in Tables A-21 to A-37 (Appendix A) were sequenced to determine the genotypes at these polymorphic sites. Tailed (Universal M13 Forward and Reverse) PCR primers were designed using the sequence of SEQ ID NO:1 for ACE, SEQ ID NO:2 for ATP5C3, SEQ ID NO:3 for BCL2L1, SEQ ID NO:4 for CYP2C9, SEQ ID NO:5 for DRD3, SEQ ID NO:6 for FOS, SEQ ID NO:7 for DTNBP1, SEQ ID NO:8 for GABRG3, SEQ ID NO:9 for GRIA4, SEQ ID NO:10 for LAMA4, SEQ ID NO:11 for MAPK1, SEQ ID NO:12 for NPY1R, SEQ ID NO:13 for OPRD1, SEQ ID NO:14 for OPRM1, SEQ ID NO:15 for PER3, SEQ ID NO:16 for PLCB1, and SEQ ID NO:17 for PSMD1. Amplified PCR products were sequenced using Applied Biosystems' Big Dye® Terminator v 3.1 cycle sequencing kit according to manufacturer's instructions. The reaction products were then electrophoresed using an Applied Biosystems 3700 or 3730xl DNA analyzer. Polymorphisms were identified using the Polyphred program, and confirmed by visual inspection.

### Example 4

This example illustrates genotyping of the study group for the ABI1, LOC402382, and NCALD polymorphic sites selected by the inventors herein for analysis. Genomic DNA (250 ng) was digested with a restriction enzyme (Nsp I or Sty I) and ligated to adaptors that recognize the cohesive four base-pair (bp) overhangs. All fragments resulting from restriction enzyme digestion, regardless of size, were substrates for adaptor ligation. A generic primer that recognized the adaptor sequence was used to amplify adaptor-ligated DNA fragments. PCR conditions were optimized to preferentially amplify fragments in the 200 to 1,100 bp size range. The amplified DNA was then fragmented, labeled, and hybridized to an Affymetrix GeneChip® Human Mapping 250K Array. All single nucleotide polymorphisms (SNPs) on the array went through a rigorous screening and validation process. Optimal SNPs were selected and tiled on arrays based on accuracy, call rate, and linkage disequilibrium analysis in three populations across the genome. The median physical distance between SNPs was 2.5 kb and the average distance between SNPs was 5.8 kb. The average heterozygosity of these SNPs was 0.30. Eighty-five percent of the human genome is within 10 kb of a SNP.

### Example 4

This example illustrates the deduction of markers from the genotyping data generated in Examples 2 and 3.

Haplotypes were estimated from the unphased genotypes using a computer-implemented algorithm for assigning haplotypes to unrelated individuals in a population sample, essentially as described in WO 01/80156. In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals.

A quality control analysis was performed on the deduced haplotypes, which included analysis of the frequencies of the haplotypes and individual SNPs therein for compliance with principles of Hardy-Weinberg equilibrium.

### Example 5

This example illustrates analysis of the markers in Tables A-1 to A-17 (Appendix A) for association with vilazodone response. A proprietary algorithm was used as a tool for finding associations between markers and outcomes. The clinical outcomes were improvement in disease over eight weeks of treatment with vilazodone as measured by the MADRS scale. A linear model was first fitted on the covariates, which were treatment center, race, ethnicity, and baseline values. The resulting residuals were used as the outcome in a t-test in which, for each haplotype being considered, the dominant or recessive mode divided the sample into two groups.

For the results obtained on the analyses, adjustments were made for multiple comparisons, using a permutation test. (See, e.g., PERMUTATION TESTS: A PRACTICAL GUIDE TO RESAMPLING METHODS FOR TESTING HYPOTHESES, 2^{nd} ed., Good, Springer Series in Statistics, New York (2000)). In this test, the marker data for each observation were kept constant, while all the remaining variables (outcome and covariates) were randomly permuted so that covariates always stayed with the same outcome. The permutation model was fitted for each of the several haplotypes, and the lowest p-value was kept. In total, up to 6250 permutations were done, depending on the level of significance. Various markers were identified that show a correlation with vilazodone response, as measured by having no more than ten percent of the lowest permutation p-values be as low or lower than the marker p-value from the original set. The unadjusted (“raw”) p-values, the differences between the mean MADRS score decreases among the patients who carry the markers compared with those who do not, and the percentages of the patients who carry the markers are shown in Tables 1-17 below.

TABLE 1
Markers in ACE Associated with Better Vilazodone Response
Measured as Change of MADRS

| | | Re- | | | | |
|---|---|---|---|---|---|---|
| | | sponse | Percent | | | Percent |
| | | Differ- | with | | Response | with |
| | | ence | Marker | p | Difference | Marker |
| | p (all | (all | (all | (Cauc. | (Cauc. | (Cauc. |
| Marker | ethnic.) | ethnic.) | ethnic.) | only) | only) | only) |
| 1 | 0.00007 | −6 | 71 | NA | NA | NA |
| 2 | 0.00035 | −5.4 | 70 | 0.000328 | −5.6 | 69 |
| 3 | 0.000083 | −5.5 | 57 | NA | NA | NA |
| 4 | 0.002791 | −4.5 | 68 | 0.003758 | −4.6 | 70 |
| 5 | 0.000035 | −5.7 | 56 | NA | NA | NA |
| 6 | 0.00011 | −5.4 | 54 | NA | NA | NA |
| 7 | 0.000067 | −5.6 | 57 | 0.007019 | −4 | 61 |
| 8 | 0.000067 | −5.6 | 57 | NA | NA | NA |
| 9 | 0.000164 | −5.5 | 65 | NA | NA | NA |
| 10 | 0.000019 | −6.1 | 63 | NA | NA | NA |
| 11 | 0.002137 | −4.6 | 67 | 0.004984 | −4.4 | 69 |
| 12 | 0.000352 | −5 | 57 | 0.017109 | −3.5 | 61 |
| 13 | 0.000401 | −4.9 | 56 | 0.008751 | −3.9 | 60 |
| 14 | 0.000125 | −6.3 | 71 | 0.000154 | −5.9 | 70 |
| 15 | 0.009284 | −4.1 | 27 | 0.003649 | −4.6 | 29 |
| 16 | 0.027298 | −3.2 | 64 | 0.004663 | −4.6 | 73 |
| 17 | 0.003483 | −5.3 | 82 | 0.000145 | −6.9 | 81 |
| 18 | 0.001236 | −5.8 | 82 | 0.000438 | −7 | 84 |
| 19 | 0.003387 | −4.7 | 74 | 0.000154 | −6 | 71 |
| 20 | 0.002075 | −5.1 | 24 | 0.000382 | −5.8 | 26 |
| 21 | 0.000874 | −5.6 | 22 | 0.000128 | −6.4 | 24 |
| 22 | 0.00104 | −5.6 | 21 | 0.000045 | −6.8 | 24 |
| 23 | 0.001961 | −5.9 | 78 | 0.000155 | −7.3 | 84 |
| 24 | 0.00271 | −5.5 | 83 | 0.000084 | −7.2 | 81 |
| 25 | 0.005992 | −4 | 64 | 0.002285 | −4.7 | 68 |
| 26 | 0.004748 | −4.7 | 78 | 0.000292 | −6.9 | 83 |
| 27 | 0.002791 | −4.5 | 68 | 0.003758 | −4.6 | 70 |
| 28 | 0.002791 | −4.5 | 68 | 0.003758 | −4.6 | 70 |
| 29 | 0.000035 | −5.7 | 56 | NA | NA | NA |
| 30 | 0.000035 | −5.7 | 56 | NA | NA | NA |
| 31 | 0.000164 | −5.5 | 65 | NA | NA | NA |
| 32 | 0.002137 | −4.6 | 67 | 0.004984 | −4.4 | 69 |
| 33 | 0.002137 | −4.6 | 67 | 0.004984 | −4.4 | 69 |
| 34 | 0.000352 | −5 | 57 | 0.017109 | −3.5 | 61 |
| 35 | 0.000401 | −4.9 | 56 | 0.008751 | −3.9 | 60 |
| 36 | 0.000401 | −4.9 | 56 | 0.008751 | −3.9 | 60 |
| 37 | 0.001961 | −5.9 | 78 | 0.000155 | −7.3 | 84 |
| 38 | 0.001961 | −5.9 | 78 | 0.000155 | −7.3 | 84 |
| 39 | 0.001961 | −5.9 | 78 | 0.000155 | −7.3 | 84 |
| 40 | 0.001961 | −5.9 | 78 | 0.000155 | −7.3 | 84 |
| 41 | 0.001961 | −5.9 | 78 | 0.000155 | −7.3 | 84 |
| 42 | 0.00271 | −5.5 | 83 | 0.000084 | −7.2 | 81 |
| 43 | 0.00271 | −5.5 | 83 | 0.000084 | −7.2 | 81 |
| 44 | 0.00271 | −5.5 | 83 | 0.000084 | −7.2 | 81 |
| 45 | 0.00271 | −5.5 | 83 | 0.000084 | −7.2 | 81 |
| 46 | 0.00271 | −5.5 | 83 | 0.000084 | −7.2 | 81 |
| 47 | 0.00271 | −5.5 | 83 | 0.000084 | −7.2 | 81 |

TABLE 2
Markers in ATP5G3 Associated with Better Vilazodone Response
Measured as Change of MADRS

| | | | Percent | | | Percent |
|---|---|---|---|---|---|---|
| | | Response | with | | Response | with |
| | | difference | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.00291 | −5.6 | 17 | 0.000367 | −6.6 | 19 |
| 2 | 0.02269 | −5.4 | 10 | 0.009542 | −5.9 | 12 |

TABLE 3
Markers in BCL2L1 associated with better Vilazodone response
measured as change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.052536 | −3.9 | 86 | 0.004809 | −6.4 | 0.88 |
| 5 | 0.052536 | −3.9 | 86 | 0.004809 | −6.4 | 88 |
| 6 | 0.052536 | −3.9 | 0.86 | 0.004809 | −6.4 | 0.88 |
| 7 | 0.052536 | −3.9 | 0.86 | 0.004809 | −6.4 | 0.88 |
| 8 | 0.052536 | −3.9 | 0.86 | 0.004809 | −6.4 | 0.88 |
| 9 | 0.052536 | −3.9 | 0.86 | 0.004809 | −6.4 | 0.88 |
| 2 | 0.321106 | −1.4 | 0.52 | 0.012807 | −3.6 | 0.53 |
| 10 | 0.321106 | −1.4 | 0.52 | 0.012807 | −3.6 | 0.53 |
| 3 | 0.174836 | −2 | 0.61 | 0.007243 | −4 | 0.6 |
| 11 | 0.174836 | −2 | 0.61 | 0.007243 | −4 | 0.6 |
| 12 | 0.174836 | −2 | 0.61 | 0.007243 | −4 | 0.6 |
| 13 | 0.174836 | −2 | 0.61 | 0.007243 | −4 | 0.6 |
| 14 | 0.174836 | −2 | 0.61 | 0.007243 | −4 | 0.6 |
| 4 | 0.174836 | −2 | 0.61 | 0.007243 | −4 | 0.6 |
| 15 | 0.174836 | −2 | 0.61 | 0.007243 | −4 | 0.6 |
| 16 | 0.174836 | −2 | 0.61 | 0.007243 | −4 | 0.6 |
| 17 | 0.174836 | −2 | 0.61 | 0.007243 | −4 | 0.6 |
| 18 | 0.174836 | −2 | 0.61 | 0.007243 | −4 | 0.6 |
| 19 | 0.174836 | −2 | 0.61 | 0.007243 | −4 | 0.6 |
| 20 | 0.174836 | −2 | 0.61 | 0.007243 | −4 | 0.6 |

TABLE 4
Markers in CYP2C9 Associated with Better Vilazodone Response
Measured as Change of MADRS

| | | | Percent | | | Percent |
|---|---|---|---|---|---|---|
| | | Response | with | | Response | with |
| | | Difference | Marker | p | Difference | Marker |
| | p (all | (all | (all | (Cauc. | (Cauc. | (Cauc. |
| Marker | ethnic.) | ethnic.) | ethnic.) | only) | only) | only) |
| 1 | 0.00097 | −6.3 | 84 | 0.00202 | −5.7 | 81 |

TABLE 5
Markers in DRD3 Associated with Better Vilazodone Response
Measured as Change of MADRS

| | | | Percent | | | Percent |
|---|---|---|---|---|---|---|
| | | Response | with | | Response | with |
| | | Difference | Marker | p | Difference | Marker |
| | p (all | (all | (all | (Cauc. | (Cauc. | (Cauc. |
| Marker | ethnic.) | ethnic.) | ethnic.) | only) | only) | only) |
| 1 | 3.3E−05 | −8.1 | 85 | NA | NA | NA |
| 2 | 0.00015 | −7.6 | 86 | 0.00191 | −6.2 | 84 |
| 7 | 0.00015 | −7.6 | 86 | 0.00191 | −6.2 | 84 |
| 8 | 0.00015 | −7.6 | 86 | 0.00191 | −6.2 | 84 |
| 3 | 0.00035 | −7.6 | 88 | NA | NA | NA |
| 9 | 0.00035 | −7.6 | 88 | NA | NA | NA |
| 4 | 5.9E−05 | −8.1 | 87 | NA | NA | NA |
| 5 | 7.7E−05 | −7.7 | 85 | NA | NA | NA |
| 6 | 0.00045 | −7.6 | 89 | NA | NA | NA |
| 10 | 0.00045 | −7.6 | 89 | NA | NA | NA |

TABLE 6
Markers in FOS Associated with Better Vilazodone Response
Measured as Change of MADRS

| | | | Percent | | | Percent |
|---|---|---|---|---|---|---|
| | | Response | with | | Response | with |
| | | Difference | Marker | p | Difference | Marker |
| | p (all | (all | (all | (Cauc. | (Cauc. | (Cauc. |
| Marker | ethnic.) | ethnic.) | ethnic.) | only) | only) | only) |
| 1 | 0.00362 | −9.2 | 95 | NA | NA | NA |
| 2 | 0.00362 | −9.2 | 95 | 0.27338 | −1.6 | 45 |
| 3 | 0.02612 | −3.3 | 68 | 0.00143 | −4.9 | 67 |
| 4 | 0.02292 | −3.4 | 68 | 0.00114 | −5 | 68 |
| 5 | 0.00962 | −4.2 | 75 | 0.00024 | −6.2 | 77 |

TABLE 7
Markers in DTNBP1 Associated with Better Vilazodone Response
Measured as Change of MADRS

| | | Re- | | | | |
|---|---|---|---|---|---|---|
| | | sponse | Percent | | | Percent |
| | | Differ- | with | | Response | with |
| | | ence | Marker | | Difference | Marker |
| | p (all | (all | (all | p (Cauc. | (Cauc. | (Cauc. |
| Marker | ethnic.) | ethnic.) | ethnic.) | only) | only) | only) |
| 1 | 0.000018 | −6.4 | 31 | 0.000027 | −6.6 | 29 |
| 2 | 0.000031 | −6.1 | 32 | 0.000041 | −6.3 | 30 |
| 3 | 0.000009 | −6.6 | 31 | NA | NA | NA |
| 4 | 0.000019 | −6.4 | 31 | 0.000012 | −6.8 | 29 |
| 5 | 0.000024 | −6.3 | 31 | NA | NA | NA |
| 6 | 0.000016 | −6.4 | 32 | NA | NA | NA |
| 12 | 0.000016 | −6.4 | 32 | NA | NA | NA |
| 13 | 0.000016 | −6.4 | 32 | NA | NA | NA |
| 14 | 0.000016 | −6.4 | 32 | NA | NA | NA |
| 15 | 0.000016 | −6.4 | 32 | NA | NA | NA |
| 16 | 0.000016 | −6.4 | 32 | NA | NA | NA |
| 17 | 0.000016 | −6.4 | 32 | NA | NA | NA |
| 18 | 0.000016 | −6.4 | 32 | NA | NA | NA |
| 19 | 0.000016 | −6.4 | 32 | NA | NA | NA |
| 20 | 0.000016 | −6.4 | 32 | NA | NA | NA |
| 21 | 0.000016 | −6.4 | 32 | NA | NA | NA |
| 7 | 0.000011 | −6.5 | 31 | 0.00001 | −6.8 | 30 |
| 8 | 0.000026 | −6.1 | 35 | 0.00008 | −5.9 | 36 |
| 9 | 0.000258 | −5.4 | 34 | 0.001033 | −5 | 34 |
| 10 | 0.000014 | −6.8 | 32 | 0.000017 | −6.7 | 29 |
| 11 | 0.000459 | −6.6 | 16 | 0.000084 | −7.9 | 15 |
| 22 | 0.000459 | −6.6 | 16 | 0.000084 | −7.9 | 15 |
| 23 | 0.000459 | −6.6 | 16 | 0.000084 | −7.9 | 15 |
| 24 | 0.000459 | −6.6 | 16 | 0.000084 | −7.9 | 15 |
| 25 | 0.000459 | −6.6 | 16 | 0.000084 | −7.9 | 15 |
| 26 | 0.000459 | −6.6 | 16 | 0.000084 | −7.9 | 15 |
| 27 | 0.000459 | −6.6 | 16 | 0.000084 | −7.9 | 15 |
| 28 | 0.000459 | −6.6 | 16 | 0.000084 | −7.9 | 15 |
| 29 | 0.000459 | −6.6 | 16 | 0.000084 | −7.9 | 15 |
| 30 | 0.000459 | −6.6 | 16 | 0.000084 | −7.9 | 15 |
| 31 | 0.000459 | −6.6 | 16 | 0.000084 | −7.9 | 15 |
| 32 | 0.000459 | −6.6 | 16 | 0.000084 | −7.9 | 15 |
| 33 | 0.000459 | −6.6 | 16 | 0.000084 | −7.9 | 15 |
| 34 | 0.000459 | −6.6 | 16 | 0.000084 | −7.9 | 15 |
| 35 | 0.000459 | −6.6 | 16 | 0.000084 | −7.9 | 15 |
| 36 | 0.000459 | −6.6 | 16 | 0.000084 | −7.9 | 15 |

TABLE 8
Markers in GABRG3 Associated with Better Vilazodone Response
Measured as Change of MADRS

| | | | Percent | | | Percent |
|---|---|---|---|---|---|---|
| | | Response | with | | Response | with |
| | | Difference | Marker | p | Difference | Marker |
| | p (all | (all | (all | (Cauc. | (Cauc. | (Cauc. |
| Marker | ethnic.) | ethnic.) | ethnic.) | only) | only) | only) |
| 1 | 0.00143 | −6.8 | 12 | 0.00015 | −7.8 | 14 |
| 10 | 0.00143 | −6.8 | 12 | 0.00015 | −7.8 | 14 |
| 11 | 0.00143 | −6.8 | 12 | 0.00015 | −7.8 | 14 |
| 12 | 0.00143 | −6.8 | 12 | 0.00015 | −7.8 | 14 |
| 13 | 0.00143 | −6.8 | 12 | 0.00015 | −7.8 | 14 |
| 2 | 0.00236 | −5.4 | 19 | 0.00035 | −6.1 | 22 |
| 3 | 0.00831 | −3.7 | 42 | 0.0003 | −5.2 | 43 |
| 4 | 0.00829 | −3.8 | 40 | 0.00029 | −5.3 | 42 |
| 5 | 0.02916 | −3.1 | 55 | 0.0025 | −4.4 | 53 |
| 6 | 0.00657 | −6 | 11 | 0.00102 | −7 | 13 |
| 7 | 0.02867 | −3.2 | 34 | 0.00328 | −4.4 | 36 |
| 8 | 0.0107 | −3.7 | 61 | 0.00035 | −5.2 | 60 |
| 9 | 0.00365 | −4.7 | 24 | 0.00029 | −5.9 | 26 |

TABLE 9
Markers in GRIA4 Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.00068 | −8 | 90 | NA | NA | NA |
| 71 | 0.00068 | −8 | 90 | NA | NA | NA |
| 72 | 0.00068 | −8 | 90 | NA | NA | NA |
| 73 | 0.00068 | −8 | 90 | NA | NA | NA |
| 74 | 0.00068 | −8 | 90 | NA | NA | NA |
| 75 | 0.00068 | −8 | 90 | NA | NA | NA |
| 76 | 0.00068 | −8 | 90 | NA | NA | NA |
| 77 | 0.00068 | −8 | 90 | NA | NA | NA |
| 2 | 0.00016 | −12.7 | 96 | 0.000614 | −11.7 | 95 |
| 78 | 0.00016 | −12.7 | 96 | 0.000614 | −11.7 | 95 |
| 3 | 1.6E−05 | −12.8 | 94 | 0.000054 | −11.9 | 94 |
| 79 | 1.6E−05 | −12.8 | 94 | 0.000054 | −11.9 | 94 |
| 4 | 6.6E−05 | −12.5 | 95 | 0.000241 | −11.6 | 95 |
| 5 | 0.00248 | −5 | 77 | 0.000307 | −6.3 | 79 |
| 6 | 0.02065 | −5 | 88 | 0.00015 | −5.5 | 43 |
| 7 | 0.03314 | −4.7 | 89 | 0.000133 | −5.5 | 44 |
| 8 | 0.00138 | −7.4 | 90 | 0.000239 | −8.7 | 90 |
| 9 | 0.00105 | −7.3 | 89 | 0.000196 | −8.5 | 89 |
| 10 | 0.01395 | −3.8 | 70 | 0.001983 | −5.1 | 74 |
| 11 | 0.00618 | −4.1 | 68 | 0.000436 | −5.6 | 71 |
| 12 | 0.04535 | −5.8 | 94 | 0.003007 | −9.4 | 95 |
| 13 | 0.01153 | −4.8 | 83 | 0.00033 | −7.2 | 85 |
| 14 | 0.01517 | −3.8 | 73 | 0.000287 | −6.2 | 78 |
| 80 | 0.01517 | −3.8 | 73 | 0.000287 | −6.2 | 78 |
| 81 | 0.01517 | −3.8 | 73 | 0.000287 | −6.2 | 78 |
| 82 | 0.01517 | −3.8 | 73 | 0.000287 | −6.2 | 78 |
| 83 | 0.01517 | −3.8 | 73 | 0.000287 | −6.2 | 78 |
| 84 | 0.01517 | −3.8 | 73 | 0.000287 | −6.2 | 78 |
| 85 | 0.01517 | −3.8 | 73 | 0.000287 | −6.2 | 78 |
| 86 | 0.01517 | −3.8 | 73 | 0.000287 | −6.2 | 78 |
| 87 | 0.01517 | −3.8 | 73 | 0.000287 | −6.2 | 78 |
| 88 | 0.01517 | −3.8 | 73 | 0.000287 | −6.2 | 78 |
| 89 | 0.01517 | −3.8 | 73 | 0.000287 | −6.2 | 78 |
| 90 | 0.01517 | −3.8 | 73 | 0.000287 | −6.2 | 78 |
| 91 | 0.01517 | −3.8 | 73 | 0.000287 | −6.2 | 78 |
| 92 | 0.01517 | −3.8 | 73 | 0.000287 | −6.2 | 78 |
| 93 | 0.01517 | −3.8 | 73 | 0.000287 | −6.2 | 78 |
| 94 | 0.01517 | −3.8 | 73 | 0.000287 | −6.2 | 78 |
| 95 | 0.01517 | −3.8 | 73 | 0.000287 | −6.2 | 78 |
| 96 | 0.01517 | −3.8 | 73 | 0.000287 | −6.2 | 78 |
| 97 | 0.01517 | −3.8 | 73 | 0.000287 | −6.2 | 78 |
| 98 | 0.01517 | −3.8 | 73 | 0.000287 | −6.2 | 78 |
| 99 | 0.01517 | −3.8 | 73 | 0.000287 | −6.2 | 78 |
| 100 | 0.01517 | −3.8 | 73 | 0.000287 | −6.2 | 78 |
| 101 | 0.01517 | −3.8 | 73 | 0.000287 | −6.2 | 78 |
| 102 | 0.01517 | −3.8 | 73 | 0.000287 | −6.2 | 78 |
| 103 | 0.01517 | −3.8 | 73 | 0.000287 | −6.2 | 78 |
| 104 | 0.01517 | −3.8 | 73 | 0.000287 | −6.2 | 78 |
| 105 | 0.01517 | −3.8 | 73 | 0.000287 | −6.2 | 78 |
| 106 | 0.01517 | −3.8 | 73 | 0.000287 | −6.2 | 78 |
| 107 | 0.01517 | −3.8 | 73 | 0.000287 | −6.2 | 78 |
| 108 | 0.01517 | −3.8 | 73 | 0.000287 | −6.2 | 78 |
| 109 | 0.01517 | −3.8 | 73 | 0.000287 | −6.2 | 78 |
| 110 | 0.01517 | −3.8 | 73 | 0.000287 | −6.2 | 78 |
| 111 | 0.01517 | −3.8 | 73 | 0.000287 | −6.2 | 78 |
| 112 | 0.01517 | −3.8 | 73 | 0.000287 | −6.2 | 78 |
| 15 | 0.0068 | −4.3 | 74 | 0.000088 | −6.8 | 78 |
| 113 | 0.0068 | −4.3 | 74 | 0.000088 | −6.8 | 78 |
| 16 | 0.00367 | −4.6 | 73 | 0.000054 | −6.8 | 77 |
| 114 | 0.00367 | −4.6 | 73 | 0.000054 | −6.8 | 77 |
| 17 | 0.0137 | −3.8 | 69 | 0.000206 | −6 | 73 |
| 115 | 0.0137 | −3.8 | 69 | 0.000206 | −6 | 73 |
| 116 | 0.0137 | −3.8 | 69 | 0.000206 | −6 | 73 |
| 117 | 0.0137 | −3.8 | 69 | 0.000206 | −6 | 73 |
| 18 | 0.00227 | −5.7 | 83 | 0.00002 | −8.7 | 86 |
| 118 | 0.00227 | −5.7 | 83 | 0.00002 | −8.7 | 86 |
| 19 | 0.00459 | −4.4 | 72 | 0.000031 | −6.9 | 76 |
| 119 | 0.00459 | −4.4 | 72 | 0.000031 | −6.9 | 76 |
| 120 | 0.00459 | −4.4 | 72 | 0.000031 | −6.9 | 76 |
| 121 | 0.00459 | −4.4 | 72 | 0.000031 | −6.9 | 76 |
| 20 | 0.00527 | −4.3 | 71 | 0.000033 | −6.8 | 75 |
| 122 | 0.00527 | −4.3 | 71 | 0.000033 | −6.8 | 75 |
| 123 | 0.00527 | −4.3 | 71 | 0.000033 | −6.8 | 75 |
| 124 | 0.00527 | −4.3 | 71 | 0.000033 | −6.8 | 75 |
| 21 | 0.00566 | −4.3 | 72 | 0.000034 | −6.9 | 76 |
| 125 | 0.00566 | −4.3 | 72 | 0.000034 | −6.9 | 76 |
| 126 | 0.00566 | −4.3 | 72 | 0.000034 | −6.9 | 76 |
| 127 | 0.00566 | −4.3 | 72 | 0.000034 | −6.9 | 76 |
| 128 | 0.00566 | −4.3 | 72 | 0.000034 | −6.9 | 76 |
| 129 | 0.00566 | −4.3 | 72 | 0.000034 | −6.9 | 76 |
| 130 | 0.00566 | −4.3 | 72 | 0.000034 | −6.9 | 76 |
| 131 | 0.00566 | −4.3 | 72 | 0.000034 | −6.9 | 76 |
| 22 | 0.00233 | −4.8 | 73 | 0.000009 | −7.4 | 77 |
| 132 | 0.00233 | −4.8 | 73 | 0.000009 | −7.4 | 77 |
| 23 | 0.01057 | −3.9 | 70 | 0.000173 | −6.1 | 74 |
| 133 | 0.01057 | −3.9 | 70 | 0.000173 | −6.1 | 74 |
| 134 | 0.01057 | −3.9 | 70 | 0.000173 | −6.1 | 74 |
| 135 | 0.01057 | −3.9 | 70 | 0.000173 | −6.1 | 74 |
| 136 | 0.01057 | −3.9 | 70 | 0.000173 | −6.1 | 74 |
| 137 | 0.01057 | −3.9 | 70 | 0.000173 | −6.1 | 74 |
| 138 | 0.01057 | −3.9 | 70 | 0.000173 | −6.1 | 74 |
| 139 | 0.01057 | −3.9 | 70 | 0.000173 | −6.1 | 74 |
| 140 | 0.01057 | −3.9 | 70 | 0.000173 | −6.1 | 74 |
| 141 | 0.01057 | −3.9 | 70 | 0.000173 | −6.1 | 74 |
| 142 | 0.01057 | −3.9 | 70 | 0.000173 | −6.1 | 74 |
| 143 | 0.01057 | −3.9 | 70 | 0.000173 | −6.1 | 74 |
| 144 | 0.01057 | −3.9 | 70 | 0.000173 | −6.1 | 74 |
| 145 | 0.01057 | −3.9 | 70 | 0.000173 | −6.1 | 74 |
| 146 | 0.01057 | −3.9 | 70 | 0.000173 | −6.1 | 74 |
| 147 | 0.01057 | −3.9 | 70 | 0.000173 | −6.1 | 74 |
| 148 | 0.01057 | −3.9 | 70 | 0.000173 | −6.1 | 74 |
| 149 | 0.01057 | −3.9 | 70 | 0.000173 | −6.1 | 74 |
| 24 | 0.01159 | −3.9 | 72 | 0.000124 | −6.4 | 76 |
| 150 | 0.01159 | −3.9 | 72 | 0.000124 | −6.4 | 76 |
| 151 | 0.01159 | −3.9 | 72 | 0.000124 | −6.4 | 76 |
| 152 | 0.01159 | −3.9 | 72 | 0.000124 | −6.4 | 76 |
| 25 | 0.01242 | −3.9 | 73 | 0.000131 | −6.5 | 77 |
| 153 | 0.01242 | −3.9 | 73 | 0.000131 | −6.5 | 77 |
| 154 | 0.01242 | −3.9 | 73 | 0.000131 | −6.5 | 77 |
| 155 | 0.01242 | −3.9 | 73 | 0.000131 | −6.5 | 77 |
| 156 | 0.01242 | −3.9 | 73 | 0.000131 | −6.5 | 77 |
| 157 | 0.01242 | −3.9 | 73 | 0.000131 | −6.5 | 77 |
| 158 | 0.01242 | −3.9 | 73 | 0.000131 | −6.5 | 77 |
| 159 | 0.01242 | −3.9 | 73 | 0.000131 | −6.5 | 77 |
| 160 | 0.01242 | −3.9 | 73 | 0.000131 | −6.5 | 77 |
| 161 | 0.01242 | −3.9 | 73 | 0.000131 | −6.5 | 77 |
| 26 | 0.01439 | −3.4 | 50 | 0.000186 | −5.3 | 53 |
| 162 | 0.01439 | −3.4 | 50 | 0.000186 | −5.3 | 53 |
| 163 | 0.01439 | −3.4 | 50 | 0.000186 | −5.3 | 53 |
| 27 | 0.00529 | −3.9 | 54 | 0.000221 | −5.3 | 54 |
| 28 | 0.01391 | −3.5 | 50 | 0.00022 | −5.3 | 53 |
| 29 | 0.03894 | −4.1 | 85 | 0.000412 | −5.1 | 41 |
| 30 | 0.03593 | −3.8 | 81 | 0.003874 | −5.4 | 81 |
| 164 | 0.03593 | −3.8 | 81 | 0.003874 | −5.4 | 81 |
| 165 | 0.03593 | −3.8 | 81 | 0.003874 | −5.4 | 81 |
| 166 | 0.03593 | −3.8 | 81 | 0.003874 | −5.4 | 81 |
| 167 | 0.03593 | −3.8 | 81 | 0.003874 | −5.4 | 81 |
| 168 | 0.03593 | −3.8 | 81 | 0.003874 | −5.4 | 81 |
| 169 | 0.03593 | −3.8 | 81 | 0.003874 | −5.4 | 81 |
| 170 | 0.03593 | −3.8 | 81 | 0.003874 | −5.4 | 81 |
| 171 | 0.03593 | −3.8 | 81 | 0.003874 | −5.4 | 81 |
| 172 | 0.03593 | −3.8 | 81 | 0.003874 | −5.4 | 81 |
| 173 | 0.03593 | −3.8 | 81 | 0.003874 | −5.4 | 81 |
| 174 | 0.03593 | −3.8 | 81 | 0.003874 | −5.4 | 81 |
| 175 | 0.03593 | −3.8 | 81 | 0.003874 | −5.4 | 81 |
| 176 | 0.03593 | −3.8 | 81 | 0.003874 | −5.4 | 81 |
| 177 | 0.03593 | −3.8 | 81 | 0.003874 | −5.4 | 81 |
| 178 | 0.03593 | −3.8 | 81 | 0.003874 | −5.4 | 81 |
| 179 | 0.03593 | −3.8 | 81 | 0.003874 | −5.4 | 81 |
| 180 | 0.03593 | −3.8 | 81 | 0.003874 | −5.4 | 81 |
| 181 | 0.03593 | −3.8 | 81 | 0.003874 | −5.4 | 81 |
| 182 | 0.03593 | −3.8 | 81 | 0.003874 | −5.4 | 81 |
| 31 | 0.04924 | −2.8 | 40 | 0.00037 | −5.2 | 42 |
| 183 | 0.04924 | −2.8 | 40 | 0.00037 | −5.2 | 42 |
| 184 | 0.04924 | −2.8 | 40 | 0.00037 | −5.2 | 42 |
| 32 | 0.00824 | −4.4 | 77 | 0.000517 | −6 | 78 |
| 33 | 0.01345 | −4.2 | 78 | 0.000442 | −6.5 | 81 |
| 34 | 0.01111 | −4 | 73 | 0.00039 | −6 | 77 |
| 35 | 0.01079 | −3.9 | 69 | 0.000321 | −5.7 | 72 |
| 185 | 0.01079 | −3.9 | 69 | 0.000321 | −5.7 | 72 |
| 36 | 0.01111 | −4 | 73 | 0.00039 | −6 | 77 |
| 37 | 0.00755 | −4.1 | 70 | 0.00016 | −6.1 | 74 |
| 186 | 0.00755 | −4.1 | 70 | 0.00016 | −6.1 | 74 |
| 187 | 0.00755 | −4.1 | 70 | 0.00016 | −6.1 | 74 |
| 188 | 0.00755 | −4.1 | 70 | 0.00016 | −6.1 | 74 |
| 189 | 0.00755 | −4.1 | 70 | 0.00016 | −6.1 | 74 |
| 190 | 0.00755 | −4.1 | 70 | 0.00016 | −6.1 | 74 |
| 191 | 0.00755 | −4.1 | 70 | 0.00016 | −6.1 | 74 |
| 192 | 0.00755 | −4.1 | 70 | 0.00016 | −6.1 | 74 |
| 193 | 0.00755 | −4.1 | 70 | 0.00016 | −6.1 | 74 |
| 194 | 0.00755 | −4.1 | 70 | 0.00016 | −6.1 | 74 |
| 195 | 0.00755 | −4.1 | 70 | 0.00016 | −6.1 | 74 |
| 38 | 0.00755 | −4.1 | 70 | 0.00016 | −6.1 | 74 |
| 39 | 0.01414 | −3.8 | 72 | 0.000366 | −6 | 76 |
| 196 | 0.01414 | −3.8 | 72 | 0.000366 | −6 | 76 |
| 40 | 0.00835 | −4.1 | 71 | 0.000165 | −6.2 | 75 |
| 197 | 0.00835 | −4.1 | 71 | 0.000165 | −6.2 | 75 |
| 41 | 0.00645 | −4.3 | 73 | 0.000176 | −6.3 | 76 |
| 42 | 0.00645 | −4.3 | 73 | 0.000176 | −6.3 | 76 |
| 43 | 0.00546 | −6 | 88 | 0.002356 | −7 | 89 |
| 198 | 0.00546 | −6 | 88 | 0.002356 | −7 | 89 |
| 199 | 0.00546 | −6 | 88 | 0.002356 | −7 | 89 |
| 200 | 0.00546 | −6 | 88 | 0.002356 | −7 | 89 |
| 201 | 0.00546 | −6 | 88 | 0.002356 | −7 | 89 |
| 202 | 0.00546 | −6 | 88 | 0.002356 | −7 | 89 |
| 203 | 0.00546 | −6 | 88 | 0.002356 | −7 | 89 |
| 204 | 0.00546 | −6 | 88 | 0.002356 | −7 | 89 |
| 205 | 0.00546 | −6 | 88 | 0.002356 | −7 | 89 |
| 206 | 0.00546 | −6 | 88 | 0.002356 | −7 | 89 |
| 207 | 0.00546 | −6 | 88 | 0.002356 | −7 | 89 |
| 208 | 0.00546 | −6 | 88 | 0.002356 | −7 | 89 |
| 209 | 0.00546 | −6 | 88 | 0.002356 | −7 | 89 |
| 210 | 0.00546 | −6 | 88 | 0.002356 | −7 | 89 |
| 211 | 0.00546 | −6 | 88 | 0.002356 | −7 | 89 |
| 212 | 0.00546 | −6 | 88 | 0.002356 | −7 | 89 |
| 213 | 0.00546 | −6 | 88 | 0.002356 | −7 | 89 |
| 214 | 0.00546 | −6 | 88 | 0.002356 | −7 | 89 |
| 215 | 0.00546 | −6 | 88 | 0.002356 | −7 | 89 |
| 216 | 0.00546 | −6 | 88 | 0.002356 | −7 | 89 |
| 217 | 0.00546 | −6 | 88 | 0.002356 | −7 | 89 |
| 218 | 0.00546 | −6 | 88 | 0.002356 | −7 | 89 |
| 219 | 0.00546 | −6 | 88 | 0.002356 | −7 | 89 |
| 220 | 0.00546 | −6 | 88 | 0.002356 | −7 | 89 |
| 221 | 0.00546 | −6 | 88 | 0.002356 | −7 | 89 |
| 44 | 0.0043 | −4.1 | 62 | 0.000136 | −5.7 | 64 |
| 45 | 0.03894 | −4.1 | 85 | 0.000412 | −5.1 | 41 |
| 46 | 0.01439 | −3.4 | 50 | 0.000186 | −5.3 | 53 |
| 47 | 0.02194 | −3.5 | 70 | 0.002551 | −4.8 | 71 |
| 48 | 0.04924 | −2.8 | 40 | 0.00037 | −5.2 | 42 |
| 49 | 0.00281 | −5 | 78 | 0.000065 | −7.1 | 80 |
| 222 | 0.00281 | −5 | 78 | 0.000065 | −7.1 | 80 |
| 223 | 0.00281 | −5 | 78 | 0.000065 | −7.1 | 80 |
| 50 | 0.00076 | −5.6 | 77 | 0.000071 | −6.9 | 78 |
| 224 | 0.00076 | −5.6 | 77 | 0.000071 | −6.9 | 78 |
| 51 | 0.00259 | −5 | 77 | 0.000208 | −6.3 | 78 |
| 225 | 0.00259 | −5 | 77 | 0.000208 | −6.3 | 78 |
| 52 | 0.00339 | −5.9 | 86 | 0.000148 | −8.2 | 88 |
| 226 | 0.00339 | −5.9 | 86 | 0.000148 | −8.2 | 88 |
| 227 | 0.00339 | −5.9 | 86 | 0.000148 | −8.2 | 88 |
| 53 | 0.00417 | −4.8 | 78 | 0.00007 | −6.9 | 79 |
| 228 | 0.00417 | −4.8 | 78 | 0.00007 | −6.9 | 79 |
| 229 | 0.00417 | −4.8 | 78 | 0.00007 | −6.9 | 79 |
| 230 | 0.00417 | −4.8 | 78 | 0.00007 | −6.9 | 79 |
| 231 | 0.00417 | −4.8 | 78 | 0.00007 | −6.9 | 79 |
| 232 | 0.00417 | −4.8 | 78 | 0.00007 | −6.9 | 79 |
| 233 | 0.00417 | −4.8 | 78 | 0.00007 | −6.9 | 79 |
| 234 | 0.00417 | −4.8 | 78 | 0.00007 | −6.9 | 79 |
| 235 | 0.00417 | −4.8 | 78 | 0.00007 | −6.9 | 79 |
| 236 | 0.00417 | −4.8 | 78 | 0.00007 | −6.9 | 79 |
| 237 | 0.00417 | −4.8 | 78 | 0.00007 | −6.9 | 79 |
| 238 | 0.00417 | −4.8 | 78 | 0.00007 | −6.9 | 79 |
| 54 | 0.002 | −6.1 | 85 | 0.000078 | −8.3 | 87 |
| 239 | 0.002 | −6.1 | 85 | 0.000078 | −8.3 | 87 |
| 240 | 0.002 | −6.1 | 85 | 0.000078 | −8.3 | 87 |
| 55 | 0.0015 | −5.4 | 78 | 0.000018 | −7.6 | 80 |
| 241 | 0.0015 | −5.4 | 78 | 0.000018 | −7.6 | 80 |
| 242 | 0.0015 | −5.4 | 78 | 0.000018 | −7.6 | 80 |
| 56 | 0.00093 | −5.6 | 78 | 0.000063 | −7 | 79 |
| 243 | 0.00093 | −5.6 | 78 | 0.000063 | −7 | 79 |
| 57 | 0.00272 | −5 | 78 | 0.000218 | −6.4 | 78 |
| 244 | 0.00272 | −5 | 78 | 0.000218 | −6.4 | 78 |
| 245 | 0.00272 | −5 | 78 | 0.000218 | −6.4 | 78 |
| 246 | 0.00272 | −5 | 78 | 0.000218 | −6.4 | 78 |
| 247 | 0.00272 | −5 | 78 | 0.000218 | −6.4 | 78 |
| 248 | 0.00272 | −5 | 78 | 0.000218 | −6.4 | 78 |
| 249 | 0.00272 | −5 | 78 | 0.000218 | −6.4 | 78 |
| 250 | 0.00272 | −5 | 78 | 0.000218 | −6.4 | 78 |
| 251 | 0.00272 | −5 | 78 | 0.000218 | −6.4 | 78 |
| 252 | 0.00272 | −5 | 78 | 0.000218 | −6.4 | 78 |
| 253 | 0.00272 | −5 | 78 | 0.000218 | −6.4 | 78 |
| 254 | 0.00272 | −5 | 78 | 0.000218 | −6.4 | 78 |
| 58 | 0.00124 | −5.3 | 77 | 0.000108 | −6.6 | 78 |
| 255 | 0.00124 | −5.3 | 77 | 0.000108 | −6.6 | 78 |
| 256 | 0.00124 | −5.3 | 77 | 0.000108 | −6.6 | 78 |
| 257 | 0.00124 | −5.3 | 77 | 0.000108 | −6.6 | 78 |
| 59 | 0.00093 | −5.6 | 78 | 0.000063 | −7 | 79 |
| 60 | 0.01072 | −4.5 | 80 | 0.000375 | −6.4 | 81 |
| 258 | 0.01072 | −4.5 | 80 | 0.000375 | −6.4 | 81 |
| 259 | 0.01072 | −4.5 | 80 | 0.000375 | −6.4 | 81 |
| 260 | 0.01072 | −4.5 | 80 | 0.000375 | −6.4 | 81 |
| 261 | 0.01072 | −4.5 | 80 | 0.000375 | −6.4 | 81 |
| 262 | 0.01072 | −4.5 | 80 | 0.000375 | −6.4 | 81 |
| 61 | 0.00184 | −5.2 | 78 | 0.000228 | −6.5 | 79 |
| 263 | 0.00184 | −5.2 | 78 | 0.000228 | −6.5 | 79 |
| 264 | 0.00184 | −5.2 | 78 | 0.000228 | −6.5 | 79 |
| 62 | 0.00518 | −4.6 | 76 | 0.000119 | −6.6 | 78 |
| 265 | 0.00518 | −4.6 | 76 | 0.000119 | −6.6 | 78 |
| 266 | 0.00518 | −4.6 | 76 | 0.000119 | −6.6 | 78 |
| 267 | 0.00518 | −4.6 | 76 | 0.000119 | −6.6 | 78 |
| 268 | 0.00518 | −4.6 | 76 | 0.000119 | −6.6 | 78 |
| 269 | 0.00518 | −4.6 | 76 | 0.000119 | −6.6 | 78 |
| 270 | 0.00518 | −4.6 | 76 | 0.000119 | −6.6 | 78 |
| 271 | 0.00518 | −4.6 | 76 | 0.000119 | −6.6 | 78 |
| 272 | 0.00518 | −4.6 | 76 | 0.000119 | −6.6 | 78 |
| 273 | 0.00518 | −4.6 | 76 | 0.000119 | −6.6 | 78 |
| 274 | 0.00518 | −4.6 | 76 | 0.000119 | −6.6 | 78 |
| 275 | 0.00518 | −4.6 | 76 | 0.000119 | −6.6 | 78 |
| 276 | 0.00518 | −4.6 | 76 | 0.000119 | −6.6 | 78 |
| 277 | 0.00518 | −4.6 | 76 | 0.000119 | −6.6 | 78 |
| 278 | 0.00518 | −4.6 | 76 | 0.000119 | −6.6 | 78 |
| 279 | 0.00518 | −4.6 | 76 | 0.000119 | −6.6 | 78 |
| 280 | 0.00518 | −4.6 | 76 | 0.000119 | −6.6 | 78 |
| 281 | 0.00518 | −4.6 | 76 | 0.000119 | −6.6 | 78 |
| 282 | 0.00518 | −4.6 | 76 | 0.000119 | −6.6 | 78 |
| 283 | 0.00518 | −4.6 | 76 | 0.000119 | −6.6 | 78 |
| 284 | 0.00518 | −4.6 | 76 | 0.000119 | −6.6 | 78 |
| 285 | 0.00518 | −4.6 | 76 | 0.000119 | −6.6 | 78 |
| 63 | 0.00547 | −4.6 | 77 | 0.000123 | −6.6 | 78 |
| 286 | 0.00547 | −4.6 | 77 | 0.000123 | −6.6 | 78 |
| 287 | 0.00547 | −4.6 | 77 | 0.000123 | −6.6 | 78 |
| 288 | 0.00547 | −4.6 | 77 | 0.000123 | −6.6 | 78 |
| 289 | 0.00547 | −4.6 | 77 | 0.000123 | −6.6 | 78 |
| 64 | 0.00209 | −5 | 76 | 0.000224 | −6.2 | 77 |
| 290 | 0.00209 | −5 | 76 | 0.000224 | −6.2 | 77 |
| 65 | 0.00345 | −5 | 78 | 0.000504 | −6.2 | 80 |
| 291 | 0.00345 | −5 | 78 | 0.000504 | −6.2 | 80 |
| 292 | 0.00345 | −5 | 78 | 0.000504 | −6.2 | 80 |
| 66 | 0.0022 | −5 | 76 | 0.000236 | −6.3 | 78 |
| 293 | 0.0022 | −5 | 76 | 0.000236 | −6.3 | 78 |
| 67 | 0.0022 | −5 | 76 | 0.000236 | −6.3 | 78 |
| 68 | 0.01185 | −3.5 | 50 | 0.000148 | −5.4 | 53 |
| 294 | 0.01185 | −3.5 | 50 | 0.000148 | −5.4 | 53 |
| 69 | 0.01178 | −4.2 | 77 | 0.000417 | −6.1 | 78 |
| 295 | 0.01178 | −4.2 | 77 | 0.000417 | −6.1 | 78 |
| 296 | 0.01178 | −4.2 | 77 | 0.000417 | −6.1 | 78 |
| 297 | 0.01178 | −4.2 | 77 | 0.000417 | −6.1 | 78 |
| 298 | 0.01178 | −4.2 | 77 | 0.000417 | −6.1 | 78 |
| 299 | 0.01178 | −4.2 | 77 | 0.000417 | −6.1 | 78 |
| 300 | 0.01178 | −4.2 | 77 | 0.000417 | −6.1 | 78 |
| 301 | 0.01178 | −4.2 | 77 | 0.000417 | −6.1 | 78 |
| 302 | 0.01178 | −4.2 | 77 | 0.000417 | −6.1 | 78 |
| 303 | 0.01178 | −4.2 | 77 | 0.000417 | −6.1 | 78 |
| 304 | 0.01178 | −4.2 | 77 | 0.000417 | −6.1 | 78 |
| 305 | 0.01178 | −4.2 | 77 | 0.000417 | −6.1 | 78 |
| 306 | 0.01178 | −4.2 | 77 | 0.000417 | −6.1 | 78 |
| 307 | 0.01178 | −4.2 | 77 | 0.000417 | −6.1 | 78 |
| 308 | 0.01178 | −4.2 | 77 | 0.000417 | −6.1 | 78 |
| 309 | 0.01178 | −4.2 | 77 | 0.000417 | −6.1 | 78 |
| 310 | 0.01178 | −4.2 | 77 | 0.000417 | −6.1 | 78 |
| 311 | 0.01178 | −4.2 | 77 | 0.000417 | −6.1 | 78 |
| 312 | 0.01178 | −4.2 | 77 | 0.000417 | −6.1 | 78 |
| 313 | 0.01178 | −4.2 | 77 | 0.000417 | −6.1 | 78 |
| 314 | 0.01178 | −4.2 | 77 | 0.000417 | −6.1 | 78 |
| 315 | 0.01178 | −4.2 | 77 | 0.000417 | −6.1 | 78 |
| 316 | 0.01178 | −4.2 | 77 | 0.000417 | −6.1 | 78 |
| 317 | 0.01178 | −4.2 | 77 | 0.000417 | −6.1 | 78 |
| 318 | 0.01178 | −4.2 | 77 | 0.000417 | −6.1 | 78 |
| 319 | 0.01178 | −4.2 | 77 | 0.000417 | −6.1 | 78 |
| 320 | 0.01178 | −4.2 | 77 | 0.000417 | −6.1 | 78 |
| 70 | 0.01247 | −4.2 | 78 | 0.000435 | −6.2 | 79 |
| 321 | 0.01247 | −4.2 | 78 | 0.000435 | −6.2 | 79 |
| 322 | 0.01247 | −4.2 | 78 | 0.000435 | −6.2 | 79 |
| 323 | 0.01247 | −4.2 | 78 | 0.000435 | −6.2 | 79 |
| 324 | 0.01247 | −4.2 | 78 | 0.000435 | −6.2 | 79 |

TABLE 10
Markers in LAMA4 Associated with Better Vilazodone Response
Measured as Change of MADRS

| | | | Percent | | | Percent |
|---|---|---|---|---|---|---|
| | | Response | with | | Response | with |
| | | difference | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.00076 | −7.2 | 88 | 0.00039 | −7.2 | 85 |
| 2 | 0.00043 | −7.2 | 87 | 0.000113 | −7.4 | 84 |
| 3 | 0.00051 | −5.9 | 78 | 0.000018 | −7.1 | 76 |
| 4 | 0.00014 | −6.7 | 81 | 0.000016 | −7.4 | 78 |
| 5 | 0.00049 | −5.3 | 71 | 0.000118 | −5.9 | 67 |
| 6 | 0.00525 | −5.3 | 84 | 0.000051 | −7.8 | 84 |
| 7 | 0.00741 | −4.5 | 77 | 0.000146 | −6.4 | 77 |
| 8 | 0.00703 | −6.8 | 92 | 0.002316 | −7.6 | 91 |
| 9 | 0.00278 | −4.5 | 69 | 0.001053 | −5 | 66 |
| 10 | 0.001 | −6.1 | 83 | 0.000021 | −8 | 83 |
| 11 | 0.38868 | −1.3 | 71 | 0.000095 | −13.2 | 95 |

TABLE 11
Markers in MAPK1 Associated with Better Vilazodone Response
Measured as Change of MADRS

| | | | Percent | | | Percent |
|---|---|---|---|---|---|---|
| | | Response | with | | Response | with |
| | | difference | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.00436 | −4 | 42 | 0.004241 | −4.1 | 47 |
| 2 | 0.00331 | −4.2 | 61 | 0.007552 | −4 | 64 |
| 3 | 0.00073 | −4.8 | 41 | 0.00162 | −4.6 | 44 |
| 4 | 0.00406 | −4.1 | 39 | 0.002776 | −4.4 | 43 |
| 5 | 0.00456 | −4.1 | 38 | 0.003404 | −4.3 | 42 |
| 9 | 0.00456 | −4.1 | 38 | 0.003404 | −4.3 | 42 |
| 10 | 0.00456 | −4.1 | 38 | 0.003404 | −4.3 | 42 |
| 11 | 0.00456 | −4.1 | 38 | 0.003404 | −4.3 | 42 |
| 12 | 0.00456 | −4.1 | 38 | 0.003404 | −4.3 | 42 |
| 13 | 0.00456 | −4.1 | 38 | 0.003404 | −4.3 | 42 |
| 14 | 0.00456 | −4.1 | 38 | 0.003404 | −4.3 | 42 |
| 15 | 0.00456 | −4.1 | 38 | 0.003404 | −4.3 | 42 |
| 16 | 0.00456 | −4.1 | 38 | 0.003404 | −4.3 | 42 |
| 17 | 0.00456 | −4.1 | 38 | 0.003404 | −4.3 | 42 |
| 18 | 0.00456 | −4.1 | 38 | 0.003404 | −4.3 | 42 |
| 6 | 0.02308 | −3.7 | 75 | 0.000998 | −5.7 | 78 |
| 7 | 0.00476 | −4.2 | 33 | 0.001246 | −4.8 | 37 |
| 8 | 0.00528 | −4.2 | 32 | 0.001524 | −4.7 | 36 |
| 19 | 0.00528 | −4.2 | 32 | 0.001524 | −4.7 | 36 |
| 20 | 0.00528 | −4.2 | 32 | 0.001524 | −4.7 | 36 |
| 21 | 0.00528 | −4.2 | 32 | 0.001524 | −4.7 | 36 |
| 22 | 0.00528 | −4.2 | 32 | 0.001524 | −4.7 | 36 |
| 23 | 0.00528 | −4.2 | 32 | 0.001524 | −4.7 | 36 |
| 24 | 0.00528 | −4.2 | 32 | 0.001524 | −4.7 | 36 |
| 25 | 0.00528 | −4.2 | 32 | 0.001524 | −4.7 | 36 |
| 26 | 0.00528 | −4.2 | 32 | 0.001524 | −4.7 | 36 |
| 27 | 0.00528 | −4.2 | 32 | 0.001524 | −4.7 | 36 |
| 28 | 0.00528 | −4.2 | 32 | 0.001524 | −4.7 | 36 |

TABLE 12
Markers in NPY1R Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.000998 | −4.7 | 41 | NA | NA | NA |
| 2 | 0.001864 | −4.4 | 44 | 0.002198 | −4.4 | 44 |
| 3 | 0.002751 | −4.2 | 53 | NA | NA | NA |
| 4 | 0.001163 | −4.6 | 41 | 0.002419 | −4.4 | 45 |
| 5 | 0.000729 | −4.7 | 48 | 0.001999 | −4.5 | 47 |
| 6 | 0.001295 | −4.5 | 45 | 0.001294 | −4.6 | 46 |
| 7 | 0.002129 | −4.3 | 45 | NA | NA | NA |
| 8 | 0.002978 | −4.2 | 59 | 0.024841 | −3.3 | 58 |
| 9 | 0.002932 | −4.2 | 52 | 0.006152 | −4 | 49 |
| 10 | 0.009254 | −3.8 | 62 | 0.055871 | −2.8 | 60 |
| 11 | 0.007104 | −3.8 | 47 | 0.006843 | −3.9 | 46 |
| 12 | 0.020973 | −3.3 | 46 | 0.007407 | −3.9 | 47 |
| 13 | 0.009157 | −3.7 | 52 | 0.006152 | −4 | 49 |
| 14 | 0.005079 | −3.9 | 48 | 0.004221 | −4.1 | 47 |

TABLE 13
Markers in OPRD1 Associated with Better Vilazodone Response
Measured as Change of MADRS

| | | Re- | | | | |
|---|---|---|---|---|---|---|
| | | sponse | Percent | | | Percent |
| | | differ- | with | | | with |
| | | ence | marker | p | Response | marker |
| | p (all | (all | (all | (Cauc. | difference | (Cauc. |
| Marker | ethnic.) | ethnic.) | ethnic.) | only) | (Cauc. only) | only) |
| 1 | 0.00049 | −5.3 | 30 | 7.2E−05 | −6.1 | 30 |
| 2 | 0.00055 | −5.5 | 74 | 0.00057 | −5.7 | 74 |
| 3 | 0.00026 | −7.4 | 13 | 0.00026 | −7.7 | 13 |
| 4 | 0.0004 | −7.7 | 11 | 0.0001 | −8.4 | 12 |
| 13 | 0.0004 | −7.7 | 11 | 0.0001 | −8.4 | 12 |
| 14 | 0.0004 | −7.7 | 11 | 0.0001 | −8.4 | 12 |
| 15 | 0.0004 | −7.7 | 11 | 0.0001 | −8.4 | 12 |
| 16 | 0.0004 | −7.7 | 11 | 0.0001 | −8.4 | 12 |
| 5 | 0.00021 | −12.5 | 4 | NA | NA | NA |
| 17 | 0.00021 | −12.5 | 4 | NA | NA | NA |
| 6 | 0.01277 | −4.2 | 78 | 0.00025 | −7.9 | 88 |
| 7 | 0.01368 | −3.5 | 41 | 0.00068 | −5 | 38 |
| 8 | 0.00466 | −4.5 | 26 | 0.00024 | −6.1 | 25 |
| 9 | 0.01637 | −5.3 | 11 | 0.00017 | −8.6 | 11 |
| 18 | 0.01637 | −5.3 | 11 | 0.00017 | −8.6 | 11 |
| 19 | 0.01637 | −5.3 | 11 | 0.00017 | −8.6 | 11 |
| 20 | 0.01637 | −5.3 | 11 | 0.00017 | −8.6 | 11 |
| 21 | 0.01637 | −5.3 | 11 | 0.00017 | −8.6 | 11 |
| 10 | 0.01075 | −4.3 | 78 | 0.00015 | −8.2 | 88 |
| 11 | 0.09967 | −2.4 | 59 | 0.00017 | −7.3 | 16 |
| 22 | 0.09967 | −2.4 | 59 | 0.00017 | −7.3 | 16 |
| 23 | 0.09967 | −2.4 | 59 | 0.00017 | −7.3 | 16 |
| 24 | 0.09967 | −2.4 | 59 | 0.00017 | −7.3 | 16 |
| 25 | 0.09967 | −2.4 | 59 | 0.00017 | −7.3 | 16 |
| 26 | 0.09967 | −2.4 | 59 | 0.00017 | −7.3 | 16 |
| 12 | 0.00157 | −4.6 | 34 | 3.6E−05 | −6.3 | 31 |

TABLE 14
Markers in OPRM1 Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.000063 | −10 | 92 | 0.015015 | −6.9 | 93 |
| 30 | 0.000063 | −10 | 92 | 0.015015 | −6.9 | 93 |
| 31 | 0.000063 | −10 | 92 | 0.015015 | −6.9 | 93 |
| 32 | 0.000063 | −10 | 92 | 0.015015 | −6.9 | 93 |
| 33 | 0.000063 | −10 | 92 | 0.015015 | −6.9 | 93 |
| 34 | 0.000063 | −10 | 92 | 0.015015 | −6.9 | 93 |
| 35 | 0.000063 | −10 | 92 | 0.015015 | −6.9 | 93 |
| 36 | 0.000063 | −10 | 92 | 0.015015 | −6.9 | 93 |
| 37 | 0.000063 | −10 | 92 | 0.015015 | −6.9 | 93 |
| 38 | 0.000063 | −10 | 92 | 0.015015 | −6.9 | 93 |
| 39 | 0.000063 | −10 | 92 | 0.015015 | −6.9 | 93 |
| 40 | 0.000063 | −10 | 92 | 0.015015 | −6.9 | 93 |
| 41 | 0.000063 | −10 | 92 | 0.015015 | −6.9 | 93 |
| 42 | 0.000063 | −10 | 92 | 0.015015 | −6.9 | 93 |
| 43 | 0.000063 | −10 | 92 | 0.015015 | −6.9 | 93 |
| 44 | 0.000063 | −10 | 92 | 0.015015 | −6.9 | 93 |
| 45 | 0.000063 | −10 | 92 | 0.015015 | −6.9 | 93 |
| 46 | 0.000063 | −10 | 92 | 0.015015 | −6.9 | 93 |
| 47 | 0.000063 | −10 | 92 | 0.015015 | −6.9 | 93 |
| 48 | 0.000063 | −10 | 92 | 0.015015 | −6.9 | 93 |
| 49 | 0.000063 | −10 | 92 | 0.015015 | −6.9 | 93 |
| 50 | 0.000063 | −10 | 92 | 0.015015 | −6.9 | 93 |
| 51 | 0.000063 | −10 | 92 | 0.015015 | −6.9 | 93 |
| 52 | 0.000063 | −10 | 92 | 0.015015 | −6.9 | 93 |
| 53 | 0.000063 | −10 | 92 | 0.015015 | −6.9 | 93 |
| 54 | 0.000063 | −10 | 92 | 0.015015 | −6.9 | 93 |
| 55 | 0.000063 | −10 | 92 | 0.015015 | −6.9 | 93 |
| 56 | 0.000063 | −10 | 92 | 0.015015 | −6.9 | 93 |
| 57 | 0.000063 | −10 | 92 | 0.015015 | −6.9 | 93 |
| 58 | 0.000063 | −10 | 92 | 0.015015 | −6.9 | 93 |
| 59 | 0.000063 | −10 | 92 | 0.015015 | −6.9 | 93 |
| 60 | 0.000063 | −10 | 92 | 0.015015 | −6.9 | 93 |
| 61 | 0.000063 | −10 | 92 | 0.015015 | −6.9 | 93 |
| 62 | 0.000063 | −10 | 92 | 0.015015 | −6.9 | 93 |
| 63 | 0.000063 | −10 | 92 | 0.015015 | −6.9 | 93 |
| 64 | 0.000063 | −10 | 92 | 0.015015 | −6.9 | 93 |
| 65 | 0.000063 | −10 | 92 | 0.015015 | −6.9 | 93 |
| 66 | 0.000063 | −10 | 92 | 0.015015 | −6.9 | 93 |
| 67 | 0.000063 | −10 | 92 | 0.015015 | −6.9 | 93 |
| 68 | 0.000063 | −10 | 92 | 0.015015 | −6.9 | 93 |
| 69 | 0.000063 | −10 | 92 | 0.015015 | −6.9 | 93 |
| 70 | 0.000063 | −10 | 92 | 0.015015 | −6.9 | 93 |
| 71 | 0.000063 | −10 | 92 | 0.015015 | −6.9 | 93 |
| 72 | 0.000063 | −10 | 92 | 0.015015 | −6.9 | 93 |
| 73 | 0.000063 | −10 | 92 | 0.015015 | −6.9 | 93 |
| 74 | 0.000063 | −10 | 92 | 0.015015 | −6.9 | 93 |
| 75 | 0.000063 | −10 | 92 | 0.015015 | −6.9 | 93 |
| 76 | 0.000063 | −10 | 92 | 0.015015 | −6.9 | 93 |
| 2 | 0.000015 | −6 | 44 | 0.000012 | −6.3 | 40 |
| 77 | 0.000015 | −6 | 44 | 0.000012 | −6.3 | 40 |
| 78 | 0.000015 | −6 | 44 | 0.000012 | −6.3 | 40 |
| 3 | 0.000004 | −6.3 | 45 | NA | NA | NA |
| 79 | 0.000004 | −6.3 | 45 | NA | NA | NA |
| 80 | 0.000004 | −6.3 | 45 | NA | NA | NA |
| 81 | 0.000004 | −6.3 | 45 | NA | NA | NA |
| 82 | 0.000004 | −6.3 | 45 | NA | NA | NA |
| 83 | 0.000004 | −6.3 | 45 | NA | NA | NA |
| 84 | 0.000004 | −6.3 | 45 | NA | NA | NA |
| 85 | 0.000004 | −6.3 | 45 | NA | NA | NA |
| 86 | 0.000004 | −6.3 | 45 | NA | NA | NA |
| 87 | 0.000004 | −6.3 | 45 | NA | NA | NA |
| 88 | 0.000004 | −6.3 | 45 | NA | NA | NA |
| 89 | 0.000004 | −6.3 | 45 | NA | NA | NA |
| 90 | 0.000004 | −6.3 | 45 | NA | NA | NA |
| 91 | 0.000004 | −6.3 | 45 | NA | NA | NA |
| 4 | 0.000003 | −6.4 | 45 | NA | NA | NA |
| 92 | 0.000003 | −6.4 | 45 | NA | NA | NA |
| 93 | 0.000003 | −6.4 | 45 | NA | NA | NA |
| 94 | 0.000003 | −6.4 | 45 | NA | NA | NA |
| 95 | 0.000003 | −6.4 | 45 | NA | NA | NA |
| 96 | 0.000003 | −6.4 | 45 | NA | NA | NA |
| 97 | 0.000003 | −6.4 | 45 | NA | NA | NA |
| 5 | 0.000002 | −6.5 | 44 | 0.000002 | −6.8 | 39 |
| 98 | 0.000002 | −6.5 | 44 | 0.000002 | −6.8 | 39 |
| 6 | 0.000001 | −6.6 | 44 | NA | NA | NA |
| 7 | 0.000018 | −5.9 | 53 | NA | NA | NA |
| 99 | 0.000018 | −5.9 | 53 | NA | NA | NA |
| 8 | 0.000007 | −6.2 | 53 | NA | NA | NA |
| 100 | 0.000007 | −6.2 | 53 | NA | NA | NA |
| 9 | 0.000005 | −6.3 | 53 | NA | NA | NA |
| 10 | 0.00001 | −6.4 | 65 | NA | NA | NA |
| 11 | 0.000012 | −6.3 | 66 | NA | NA | NA |
| 12 | 0.000014 | −6 | 52 | 0.000026 | −6 | 50 |
| 13 | 0.000008 | −6.1 | 54 | NA | NA | NA |
| 101 | 0.000008 | −6.1 | 54 | NA | NA | NA |
| 14 | 0.000028 | −5.8 | 54 | 0.000016 | −6.1 | 50 |
| 102 | 0.000028 | −5.8 | 54 | 0.000016 | −6.1 | 50 |
| 15 | 0.00002 | −5.9 | 54 | NA | NA | NA |
| 16 | 0.000022 | −5.8 | 52 | 0.000053 | −5.7 | 50 |
| 17 | 0.001307 | −4.5 | 46 | 0.001085 | −4.7 | 45 |
| 18 | 0.000003 | −7 | 45 | 0.000004 | −6.6 | 40 |
| 19 | 0.000033 | −5.8 | 43 | 0.000007 | −6.5 | 40 |
| 20 | 0.000017 | −6.3 | 54 | 0.00001 | −6.2 | 50 |
| 103 | 0.000017 | −6.3 | 54 | 0.00001 | −6.2 | 50 |
| 104 | 0.000017 | −6.3 | 54 | 0.00001 | −6.2 | 50 |
| 105 | 0.000017 | −6.3 | 54 | 0.00001 | −6.2 | 50 |
| 21 | 0.000107 | −5.4 | 52 | 0.000049 | −5.8 | 51 |
| 22 | 0.000084 | −5.4 | 50 | 0.000069 | −5.7 | 48 |
| 23 | 0.00022 | −5.1 | 48 | 0.000027 | −6 | 48 |
| 24 | 0.000113 | −5.3 | 50 | 0.000035 | −5.9 | 50 |
| 25 | 0.000031 | −6.1 | 54 | 0.000016 | −6.1 | 50 |
| 26 | 0.000013 | −6.4 | 54 | 0.000018 | −6.1 | 50 |
| 106 | 0.000013 | −6.4 | 54 | 0.000018 | −6.1 | 50 |
| 27 | 0.000006 | −6.6 | 55 | 0.000007 | −6.3 | 51 |
| 107 | 0.000006 | −6.6 | 55 | 0.000007 | −6.3 | 51 |
| 28 | 0.000092 | −5.4 | 54 | 0.000037 | −5.9 | 51 |
| 108 | 0.000092 | −5.4 | 54 | 0.000037 | −5.9 | 51 |
| 29 | 0.000029 | −6.1 | 55 | 0.000017 | −6.1 | 52 |

TABLE 15
Markers in PER3 Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.000034 | −6.3 | 29 | 0.000018 | −6.6 | 30 |
| 23 | 0.000034 | −6.3 | 29 | 0.000018 | −6.6 | 30 |
| 24 | 0.000034 | −6.3 | 29 | 0.000018 | −6.6 | 30 |
| 25 | 0.000034 | −6.3 | 29 | 0.000018 | −6.6 | 30 |
| 26 | 0.000034 | −6.3 | 29 | 0.000018 | −6.6 | 30 |
| 27 | 0.000034 | −6.3 | 29 | 0.000018 | −6.6 | 30 |
| 28 | 0.000034 | −6.3 | 29 | 0.000018 | −6.6 | 30 |
| 29 | 0.000034 | −6.3 | 29 | 0.000018 | −6.6 | 30 |
| 30 | 0.000034 | −6.3 | 29 | 0.000018 | −6.6 | 30 |
| 2 | 0.000195 | −5.8 | 27 | 0.000169 | −6 | 28 |
| 31 | 0.000195 | −5.8 | 27 | 0.000169 | −6 | 28 |
| 32 | 0.000195 | −5.8 | 27 | 0.000169 | −6 | 28 |
| 3 | 0.001412 | −7 | 11 | 0.002178 | −6.7 | 12 |
| 33 | 0.001412 | −7 | 11 | 0.002178 | −6.7 | 12 |
| 34 | 0.001412 | −7 | 11 | 0.002178 | −6.7 | 12 |
| 35 | 0.001412 | −7 | 11 | 0.002178 | −6.7 | 12 |
| 36 | 0.001412 | −7 | 11 | 0.002178 | −6.7 | 12 |
| 37 | 0.001412 | −7 | 11 | 0.002178 | −6.7 | 12 |
| 38 | 0.001412 | −7 | 11 | 0.002178 | −6.7 | 12 |
| 39 | 0.001412 | −7 | 11 | 0.002178 | −6.7 | 12 |
| 40 | 0.001412 | −7 | 11 | 0.002178 | −6.7 | 12 |
| 41 | 0.001412 | −7 | 11 | 0.002178 | −6.7 | 12 |
| 42 | 0.001412 | −7 | 11 | 0.002178 | −6.7 | 12 |
| 43 | 0.001412 | −7 | 11 | 0.002178 | −6.7 | 12 |
| 44 | 0.001412 | −7 | 11 | 0.002178 | −6.7 | 12 |
| 45 | 0.001412 | −7 | 11 | 0.002178 | −6.7 | 12 |
| 46 | 0.001412 | −7 | 11 | 0.002178 | −6.7 | 12 |
| 47 | 0.001412 | −7 | 11 | 0.002178 | −6.7 | 12 |
| 48 | 0.001412 | −7 | 11 | 0.002178 | −6.7 | 12 |
| 49 | 0.001412 | −7 | 11 | 0.002178 | −6.7 | 12 |
| 50 | 0.001412 | −7 | 11 | 0.002178 | −6.7 | 12 |
| 51 | 0.001412 | −7 | 11 | 0.002178 | −6.7 | 12 |
| 52 | 0.001412 | −7 | 11 | 0.002178 | −6.7 | 12 |
| 53 | 0.001412 | −7 | 11 | 0.002178 | −6.7 | 12 |
| 54 | 0.001412 | −7 | 11 | 0.002178 | −6.7 | 12 |
| 55 | 0.001412 | −7 | 11 | 0.002178 | −6.7 | 12 |
| 56 | 0.001412 | −7 | 11 | 0.002178 | −6.7 | 12 |
| 57 | 0.001412 | −7 | 11 | 0.002178 | −6.7 | 12 |
| 58 | 0.001412 | −7 | 11 | 0.002178 | −6.7 | 12 |
| 59 | 0.001412 | −7 | 11 | 0.002178 | −6.7 | 12 |
| 60 | 0.001412 | −7 | 11 | 0.002178 | −6.7 | 12 |
| 61 | 0.001412 | −7 | 11 | 0.002178 | −6.7 | 12 |
| 62 | 0.001412 | −7 | 11 | 0.002178 | −6.7 | 12 |
| 63 | 0.001412 | −7 | 11 | 0.002178 | −6.7 | 12 |
| 64 | 0.001412 | −7 | 11 | 0.002178 | −6.7 | 12 |
| 65 | 0.001412 | −7 | 11 | 0.002178 | −6.7 | 12 |
| 66 | 0.001412 | −7 | 11 | 0.002178 | −6.7 | 12 |
| 67 | 0.001412 | −7 | 11 | 0.002178 | −6.7 | 12 |
| 68 | 0.001412 | −7 | 11 | 0.002178 | −6.7 | 12 |
| 4 | 0.000063 | −6.1 | 28 | 0.00004 | −6.4 | 29 |
| 69 | 0.000063 | −6.1 | 28 | 0.00004 | −6.4 | 29 |
| 70 | 0.000063 | −6.1 | 28 | 0.00004 | −6.4 | 29 |
| 5 | 0.000207 | −5.9 | 25 | 0.000187 | −5.9 | 28 |
| 71 | 0.000207 | −5.9 | 25 | 0.000187 | −5.9 | 28 |
| 6 | 0.000227 | −5.6 | 29 | 0.000205 | −5.8 | 30 |
| 72 | 0.000227 | −5.6 | 29 | 0.000205 | −5.8 | 30 |
| 73 | 0.000227 | −5.6 | 29 | 0.000205 | −5.8 | 30 |
| 7 | 0.000346 | −5.6 | 27 | 0.000091 | −6.2 | 29 |
| 74 | 0.000346 | −5.6 | 27 | 0.000091 | −6.2 | 29 |
| 75 | 0.000346 | −5.6 | 27 | 0.000091 | −6.2 | 29 |
| 8 | 0.001412 | −7 | 11 | 0.002178 | −6.7 | 12 |
| 76 | 0.001412 | −7 | 11 | 0.002178 | −6.7 | 12 |
| 77 | 0.001412 | −7 | 11 | 0.002178 | −6.7 | 12 |
| 78 | 0.001412 | −7 | 11 | 0.002178 | −6.7 | 12 |
| 79 | 0.001412 | −7 | 11 | 0.002178 | −6.7 | 12 |
| 9 | 0.000181 | −5.8 | 28 | 0.000034 | −6.4 | 30 |
| 80 | 0.000181 | −5.8 | 28 | 0.000034 | −6.4 | 30 |
| 81 | 0.000181 | −5.8 | 28 | 0.000034 | −6.4 | 30 |
| 10 | 0.000245 | −5.5 | 31 | 0.000349 | −5.5 | 31 |
| 82 | 0.000245 | −5.5 | 31 | 0.000349 | −5.5 | 31 |
| 11 | 0.000207 | −5.9 | 25 | NA | NA | NA |
| 12 | 0.000402 | −5.8 | 24 | 0.000374 | −5.8 | 26 |
| 83 | 0.000402 | −5.8 | 24 | 0.000374 | −5.8 | 26 |
| 84 | 0.000402 | −5.8 | 24 | 0.000374 | −5.8 | 26 |
| 13 | 0.000402 | −5.8 | 24 | 0.000374 | −5.8 | 26 |
| 14 | 0.000621 | −5.1 | 32 | 0.000756 | −5.2 | 33 |
| 85 | 0.000621 | −5.1 | 32 | 0.000756 | −5.2 | 33 |
| 86 | 0.000621 | −5.1 | 32 | 0.000756 | −5.2 | 33 |
| 87 | 0.000621 | −5.1 | 32 | 0.000756 | −5.2 | 33 |
| 88 | 0.000621 | −5.1 | 32 | 0.000756 | −5.2 | 33 |
| 89 | 0.000621 | −5.1 | 32 | 0.000756 | −5.2 | 33 |
| 15 | 0.247055 | −1.8 | 73 | 0.000187 | −5.9 | 28 |
| 16 | 0.000477 | −5.5 | 26 | 0.000187 | −5.9 | 28 |
| 90 | 0.000477 | −5.5 | 26 | 0.000187 | −5.9 | 28 |
| 91 | 0.000477 | −5.5 | 26 | 0.000187 | −5.9 | 28 |
| 17 | 0.000477 | −5.5 | 26 | 0.000187 | −5.9 | 28 |
| 18 | 0.000346 | −5.6 | 27 | 0.000091 | −6.2 | 29 |
| 19 | 0.000436 | −5.5 | 27 | 0.000155 | −6 | 29 |
| 92 | 0.000436 | −5.5 | 27 | 0.000155 | −6 | 29 |
| 93 | 0.000436 | −5.5 | 27 | 0.000155 | −6 | 29 |
| 20 | 0.000886 | −5.3 | 26 | 0.000299 | −5.8 | 28 |
| 21 | 0.00034 | −5.7 | 26 | 0.000343 | −5.8 | 27 |
| 22 | 0.0016 | −5.1 | 25 | 0.000704 | −5.5 | 26 |

TABLE 16
Markers in PLCB1 Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.001904 | −4.8 | 28 | 0.000135 | −6.1 | 27 |
| 23 | 0.001904 | −4.8 | 28 | 0.000135 | −6.1 | 27 |
| 2 | 0.001928 | −4.8 | 27 | 0.000132 | −6.2 | 26 |
| 24 | 0.001928 | −4.8 | 27 | 0.000132 | −6.2 | 26 |
| 3 | 0.002002 | −5.1 | 24 | 0.000162 | −6.5 | 22 |
| 25 | 0.002002 | −5.1 | 24 | 0.000162 | −6.5 | 22 |
| 26 | 0.002002 | −5.1 | 24 | 0.000162 | −6.5 | 22 |
| 27 | 0.002002 | −5.1 | 24 | 0.000162 | −6.5 | 22 |
| 28 | 0.002002 | −5.1 | 24 | 0.000162 | −6.5 | 22 |
| 29 | 0.002002 | −5.1 | 24 | 0.000162 | −6.5 | 22 |
| 30 | 0.002002 | −5.1 | 24 | 0.000162 | −6.5 | 22 |
| 31 | 0.002002 | −5.1 | 24 | 0.000162 | −6.5 | 22 |
| 32 | 0.002002 | −5.1 | 24 | 0.000162 | −6.5 | 22 |
| 33 | 0.002002 | −5.1 | 24 | 0.000162 | −6.5 | 22 |
| 34 | 0.002002 | −5.1 | 24 | 0.000162 | −6.5 | 22 |
| 35 | 0.002002 | −5.1 | 24 | 0.000162 | −6.5 | 22 |
| 36 | 0.002002 | −5.1 | 24 | 0.000162 | −6.5 | 22 |
| 37 | 0.002002 | −5.1 | 24 | 0.000162 | −6.5 | 22 |
| 38 | 0.002002 | −5.1 | 24 | 0.000162 | −6.5 | 22 |
| 39 | 0.002002 | −5.1 | 24 | 0.000162 | −6.5 | 22 |
| 4 | 0.001985 | −5.1 | 23 | 0.000151 | −6.6 | 21 |
| 40 | 0.001985 | −5.1 | 23 | 0.000151 | −6.6 | 21 |
| 41 | 0.001985 | −5.1 | 23 | 0.000151 | −6.6 | 21 |
| 42 | 0.001985 | −5.1 | 23 | 0.000151 | −6.6 | 21 |
| 43 | 0.001985 | −5.1 | 23 | 0.000151 | −6.6 | 21 |
| 44 | 0.001985 | −5.1 | 23 | 0.000151 | −6.6 | 21 |
| 45 | 0.001985 | −5.1 | 23 | 0.000151 | −6.6 | 21 |
| 46 | 0.001985 | −5.1 | 23 | 0.000151 | −6.6 | 21 |
| 47 | 0.001985 | −5.1 | 23 | 0.000151 | −6.6 | 21 |
| 48 | 0.001985 | −5.1 | 23 | 0.000151 | −6.6 | 21 |
| 49 | 0.001985 | −5.1 | 23 | 0.000151 | −6.6 | 21 |
| 50 | 0.001985 | −5.1 | 23 | 0.000151 | −6.6 | 21 |
| 51 | 0.001985 | −5.1 | 23 | 0.000151 | −6.6 | 21 |
| 52 | 0.001985 | −5.1 | 23 | 0.000151 | −6.6 | 21 |
| 53 | 0.001985 | −5.1 | 23 | 0.000151 | −6.6 | 21 |
| 54 | 0.001985 | −5.1 | 23 | 0.000151 | −6.6 | 21 |
| 5 | 0.004619 | −4.3 | 31 | 0.000152 | −5.9 | 29 |
| 55 | 0.004619 | −4.3 | 31 | 0.000152 | −5.9 | 29 |
| 6 | 0.004747 | −4.3 | 31 | 0.000152 | −6 | 29 |
| 56 | 0.004747 | −4.3 | 31 | 0.000152 | −6 | 29 |
| 57 | 0.004747 | −4.3 | 31 | 0.000152 | −6 | 29 |
| 7 | 0.008926 | −3.9 | 33 | 0.000491 | −5.4 | 31 |
| 58 | 0.008926 | −3.9 | 33 | 0.000491 | −5.4 | 31 |
| 8 | 0.004747 | −4.3 | 31 | 0.000152 | −6 | 29 |
| 9 | 0.000908 | −5.1 | 28 | 0.00007 | −6.4 | 27 |
| 59 | 0.000908 | −5.1 | 28 | 0.00007 | −6.4 | 27 |
| 10 | 0.002695 | −4.6 | 29 | 0.000259 | −5.8 | 29 |
| 60 | 0.002695 | −4.6 | 29 | 0.000259 | −5.8 | 29 |
| 11 | 0.000915 | −5.2 | 27 | 0.000068 | −6.4 | 26 |
| 61 | 0.000915 | −5.2 | 27 | 0.000068 | −6.4 | 26 |
| 12 | 0.000662 | −5.6 | 24 | 0.000048 | −7 | 22 |
| 62 | 0.000662 | −5.6 | 24 | 0.000048 | −7 | 22 |
| 63 | 0.000662 | −5.6 | 24 | 0.000048 | −7 | 22 |
| 64 | 0.000662 | −5.6 | 24 | 0.000048 | −7 | 22 |
| 65 | 0.000662 | −5.6 | 24 | 0.000048 | −7 | 22 |
| 66 | 0.000662 | −5.6 | 24 | 0.000048 | −7 | 22 |
| 67 | 0.000662 | −5.6 | 24 | 0.000048 | −7 | 22 |
| 68 | 0.000662 | −5.6 | 24 | 0.000048 | −7 | 22 |
| 13 | 0.002246 | −4.9 | 25 | 0.000224 | −6.2 | 23 |
| 69 | 0.002246 | −4.9 | 25 | 0.000224 | −6.2 | 23 |
| 70 | 0.002246 | −4.9 | 25 | 0.000224 | −6.2 | 23 |
| 71 | 0.002246 | −4.9 | 25 | 0.000224 | −6.2 | 23 |
| 72 | 0.002246 | −4.9 | 25 | 0.000224 | −6.2 | 23 |
| 73 | 0.002246 | −4.9 | 25 | 0.000224 | −6.2 | 23 |
| 74 | 0.002246 | −4.9 | 25 | 0.000224 | −6.2 | 23 |
| 75 | 0.002246 | −4.9 | 25 | 0.000224 | −6.2 | 23 |
| 14 | 0.000648 | −5.6 | 23 | 0.000044 | −7.1 | 21 |
| 76 | 0.000648 | −5.6 | 23 | 0.000044 | −7.1 | 21 |
| 77 | 0.000648 | −5.6 | 23 | 0.000044 | −7.1 | 21 |
| 78 | 0.000648 | −5.6 | 23 | 0.000044 | −7.1 | 21 |
| 79 | 0.000648 | −5.6 | 23 | 0.000044 | −7.1 | 21 |
| 80 | 0.000648 | −5.6 | 23 | 0.000044 | −7.1 | 21 |
| 81 | 0.000648 | −5.6 | 23 | 0.000044 | −7.1 | 21 |
| 82 | 0.000648 | −5.6 | 23 | 0.000044 | −7.1 | 21 |
| 15 | 0.015084 | −4 | 24 | 0.000253 | −6.5 | 20 |
| 83 | 0.015084 | −4 | 24 | 0.000253 | −6.5 | 20 |
| 84 | 0.015084 | −4 | 24 | 0.000253 | −6.5 | 20 |
| 85 | 0.015084 | −4 | 24 | 0.000253 | −6.5 | 20 |
| 16 | 0.017091 | −4.2 | 20 | 0.000227 | −7.3 | 16 |
| 86 | 0.017091 | −4.2 | 20 | 0.000227 | −7.3 | 16 |
| 87 | 0.017091 | −4.2 | 20 | 0.000227 | −7.3 | 16 |
| 88 | 0.017091 | −4.2 | 20 | 0.000227 | −7.3 | 16 |
| 89 | 0.017091 | −4.2 | 20 | 0.000227 | −7.3 | 16 |
| 90 | 0.017091 | −4.2 | 20 | 0.000227 | −7.3 | 16 |
| 91 | 0.017091 | −4.2 | 20 | 0.000227 | −7.3 | 16 |
| 92 | 0.017091 | −4.2 | 20 | 0.000227 | −7.3 | 16 |
| 93 | 0.017091 | −4.2 | 20 | 0.000227 | −7.3 | 16 |
| 94 | 0.017091 | −4.2 | 20 | 0.000227 | −7.3 | 16 |
| 95 | 0.017091 | −4.2 | 20 | 0.000227 | −7.3 | 16 |
| 96 | 0.017091 | −4.2 | 20 | 0.000227 | −7.3 | 16 |
| 97 | 0.017091 | −4.2 | 20 | 0.000227 | −7.3 | 16 |
| 98 | 0.017091 | −4.2 | 20 | 0.000227 | −7.3 | 16 |
| 99 | 0.017091 | −4.2 | 20 | 0.000227 | −7.3 | 16 |
| 100 | 0.017091 | −4.2 | 20 | 0.000227 | −7.3 | 16 |
| 17 | 0.002639 | −4.7 | 27 | 0.00023 | −6 | 26 |
| 101 | 0.002639 | −4.7 | 27 | 0.00023 | −6 | 26 |
| 18 | 0.002669 | −4.7 | 27 | 0.000226 | −6 | 26 |
| 102 | 0.002669 | −4.7 | 27 | 0.000226 | −6 | 26 |
| 19 | 0.003485 | −4.6 | 26 | 0.000093 | −6.6 | 23 |
| 103 | 0.003485 | −4.6 | 26 | 0.000093 | −6.6 | 23 |
| 104 | 0.003485 | −4.6 | 26 | 0.000093 | −6.6 | 23 |
| 105 | 0.003485 | −4.6 | 26 | 0.000093 | −6.6 | 23 |
| 106 | 0.003485 | −4.6 | 26 | 0.000093 | −6.6 | 23 |
| 107 | 0.003485 | −4.6 | 26 | 0.000093 | −6.6 | 23 |
| 108 | 0.003485 | −4.6 | 26 | 0.000093 | −6.6 | 23 |
| 109 | 0.003485 | −4.6 | 26 | 0.000093 | −6.6 | 23 |
| 20 | 0.003514 | −4.7 | 25 | 0.000088 | −6.7 | 22 |
| 110 | 0.003514 | −4.7 | 25 | 0.000088 | −6.7 | 22 |
| 111 | 0.003514 | −4.7 | 25 | 0.000088 | −6.7 | 22 |
| 112 | 0.003514 | −4.7 | 25 | 0.000088 | −6.7 | 22 |
| 113 | 0.003514 | −4.7 | 25 | 0.000088 | −6.7 | 22 |
| 114 | 0.003514 | −4.7 | 25 | 0.000088 | −6.7 | 22 |
| 115 | 0.003514 | −4.7 | 25 | 0.000088 | −6.7 | 22 |
| 116 | 0.003514 | −4.7 | 25 | 0.000088 | −6.7 | 22 |
| 117 | 0.003514 | −4.7 | 25 | 0.000088 | −6.7 | 22 |
| 118 | 0.003514 | −4.7 | 25 | 0.000088 | −6.7 | 22 |
| 119 | 0.003514 | −4.7 | 25 | 0.000088 | −6.7 | 22 |
| 120 | 0.003514 | −4.7 | 25 | 0.000088 | −6.7 | 22 |
| 121 | 0.003514 | −4.7 | 25 | 0.000088 | −6.7 | 22 |
| 122 | 0.003514 | −4.7 | 25 | 0.000088 | −6.7 | 22 |
| 123 | 0.003514 | −4.7 | 25 | 0.000088 | −6.7 | 22 |
| 21 | 0.007488 | −4.2 | 28 | 0.000379 | −5.9 | 25 |
| 124 | 0.007488 | −4.2 | 28 | 0.000379 | −5.9 | 25 |
| 125 | 0.007488 | −4.2 | 28 | 0.000379 | −5.9 | 25 |
| 126 | 0.007488 | −4.2 | 28 | 0.000379 | −5.9 | 25 |
| 127 | 0.007488 | −4.2 | 28 | 0.000379 | −5.9 | 25 |
| 128 | 0.007488 | −4.2 | 28 | 0.000379 | −5.9 | 25 |
| 129 | 0.007488 | −4.2 | 28 | 0.000379 | −5.9 | 25 |
| 130 | 0.007488 | −4.2 | 28 | 0.000379 | −5.9 | 25 |
| 22 | 0.003514 | −4.7 | 25 | 0.000088 | −6.7 | 22 |

TABLE 17
Markers in PSMD1 Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.006514 | −7.2 | 8 | 0.109125 | −4.6 | 7 |
| 2 | 0.000086 | −10.6 | 93 | 0.409365 | −1.6 | 18 |
| 13 | 0.000086 | −10.6 | 93 | 0.409365 | −1.6 | 18 |
| 14 | 0.000086 | −10.6 | 93 | 0.409365 | −1.6 | 18 |
| 3 | 0.000629 | −10.2 | 94 | 0.333402 | −1.9 | 16 |
| 15 | 0.000629 | −10.2 | 94 | 0.333402 | −1.9 | 16 |
| 4 | 0.000086 | −10.6 | 93 | 0.171556 | −2.6 | 19 |
| 5 | 0.000418 | −7.9 | 89 | 0.145392 | −4.2 | 93 |
| 6 | 0.000323 | −7.8 | 89 | 0.145392 | −4.2 | 93 |
| 7 | 0.000323 | −7.8 | 89 | 0.339199 | −1.4 | 44 |
| 8 | 0.000713 | −6.3 | 83 | 0.170909 | −3.1 | 88 |
| 9 | 0.000666 | −5.2 | 71 | 0.127275 | −2.6 | 75 |
| 10 | 0.000553 | −9.8 | 94 | NA | NA | NA |
| 16 | 0.000553 | −9.8 | 94 | NA | NA | NA |
| 17 | 0.000553 | −9.8 | 94 | NA | NA | NA |
| 18 | 0.000553 | −9.8 | 94 | NA | NA | NA |
| 19 | 0.000553 | −9.8 | 94 | NA | NA | NA |
| 20 | 0.000553 | −9.8 | 94 | NA | NA | NA |
| 11 | 0.000381 | −7 | 85 | 0.188158 | −3.2 | 90 |
| 12 | 0.000561 | −5.5 | 75 | 0.165732 | −2.5 | 78 |

### Example 6

This example illustrates analysis of the markers in Tables A-18 to A-20 (Appendix A) for association with vilazodone response. The clinical outcomes were improvement in disease over eight weeks of treatment with vilazodone as measured by the MADRS scale. A linear model was first fitted on the covariates, which were treatment center, race, ethnicity, and baseline values. The resulting residuals were used as the outcome in a linear regression analysis in which, for each polymorphism being considered, the genotype of each patient treated with vilazodone was turned into zero, one, or two as the copy number of a reference allele. For top polymorphisms from the analysis, the dominant or recessive mode was further used to define the marker and divide the sample into two groups. The unadjusted (“raw”) p-values, the differences between the mean MADRS score decreases among the patients who carry the markers compared with those who do not, and the percentages of the patients who carry the markers are shown in Tables 18-20 below.

TABLE 18
Markers in ABI1 Associated with Better Vilazodone Response
Measured as Change of MADRS

| | | Re- | | | | |
|---|---|---|---|---|---|---|
| | | sponse | Percent | | | Percent |
| | | Differ- | with | | Response | with |
| | | ence | Marker | | Difference | Marker |
| | p (all | (all | (all | p (Cauc. | (Cauc. | (Cauc. |
| Marker | ethnic.) | ethnic.) | ethnic.) | only) | only) | only) |
| 1 | 0.000002 | −7.5 | 59 | 0.000242 | −6.1 | 64 |
| 2 | 0.000008 | −7.0 | 61 | 0.000312 | −6.0 | 65 |
| 3 | 0.000018 | −6.9 | 63 | 0.001956 | −5.2 | 66 |

TABLE 19
Markers in LOC402382 Associated with Better Vilazodone
Response Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.000984 | −5.1 | 57 | 0.000032 | −6.5 | 58 |

TABLE 20
Markers in NCALD Associated with Better Vilazodone Response
Measured as Change of MADRS

| | | Re- | | | | |
|---|---|---|---|---|---|---|
| | | sponse | Percent | | | Percent |
| | | differ- | with | | Response | with |
| | | ence | marker | | difference | marker |
| | p (all | (all | (all | p (Cauc. | (Cauc. | (Cauc. |
| Marker | ethnic.) | ethnic.) | ethnic.) | only) | only) | only) |
| 1 | 0.000022 | −6.5 | 37 | 0.000188 | −5.9 | 38 |

In view of the above, it will be seen that the several advantages of the invention are achieved and other advantageous results attained. As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications are hereby incorporated in their entirety by reference. The discussion of references herein is intended merely to summarize the assertions made by their authors and no admission is made that any reference constitutes prior art. Applicants reserve the right to challenge the accuracy and pertinence of the cited references.

### APPENDIX A

TABLE A-1
Markers in ACE Gene Associated with
Better Antidepressant Response

| | | Copy |
|---|---|---|
| Marker | Polymorphisms | Number |
| 1 | PS29-C, PS32-C, PS36-C | 0 |
| 2 | PS32-C, PS36-C | 0 |
| 3 | PS5-G, PS8-C, PS35-T | 1 OR 2 |
| 4 | PS6-C | 1 OR 2 |
| 5 | PS6-C, PS25-C, PS35-T | 1 OR 2 |
| 6 | PS6-C, PS26-C, PS35-T | 1 OR 2 |
| 7 | PS6-C, PS35-T | 1 OR 2 |
| 8 | PS6-C, PS10-C, PS35-T | 1 OR 2 |
| 9 | PS8-C, PS16-C, PS33-T | 1 OR 2 |
| 10 | PS8-C, PS16-C, PS35-T | 1 OR 2 |
| 11 | PS3-T | 1 OR 2 |
| 12 | PS3-T, PS33-T | 1 OR 2 |
| 13 | PS3-T, PS35-T | 1 OR 2 |
| 14 | PS28-C, PS32-C, PS36-C | 0 |
| 15 | PS32-C | 0 |
| 16 | PS37-C | 1 OR 2 |
| 17 | PS5-A, PS36-C, PS37-T | 0 |
| 18 | PS6-G, PS37-T | 0 OR 1 |
| 19 | PS8-C, PS32-C, PS36-C | 0 |
| 20 | PS8-C, PS32-T | 2 |
| 21 | PS8-C, PS10-C, PS32-T | 2 |
| 22 | PS8-C, PS14-G, PS32-T | 2 |
| 23 | PS2-G, PS4-T, PS37-T | 0 OR 1 |
| 24 | PS16-T, PS36-C, PS37-T | 0 |
| 25 | PS4-C | 1 OR 2 |
| 26 | PS4-T, PS37-T | 0 OR 1 |
| 27 | PS5-G, PS8-C, PS24-G | 1 OR 2 |
| 28 | PS1-G, PS5-G, PS8-C | 1 OR 2 |
| 29 | PS6-C, PS27-C, PS35-T | 1 OR 2 |
| 30 | PS6-C, PS30-G, PS35-T | 1 OR 2 |
| 31 | PS8-C, PS16-C, PS34-C | 1 OR 2 |
| 32 | PS6-C, PS7-C, PS9-C | 1 OR 2 |
| 33 | PS6-C, PS7-C, PS12-G | 1 OR 2 |
| 34 | PS3-T, PS34-C | 1 OR 2 |
| 35 | PS3-T, PS11-C, PS33-T | 1 OR 2 |
| 36 | PS3-T, PS11-C, PS34-C | 1 OR 2 |
| 37 | PS4-T, PS31-C, PS37-T | 2 |
| 38 | PS4-T, PS7-C, PS37-T | 2 |
| 39 | PS4-T, PS13-C, PS37-T | 2 |
| 40 | PS4-T, PS15-C, PS37-T | 2 |
| 41 | PS4-T, PS17-G, PS37-T | 2 |
| 42 | PS18-G, PS36-C, PS37-T | 1 OR 2 |
| 43 | PS19-C, PS36-C, PS37-T | 1 OR 2 |
| 44 | PS20-C, PS36-C, PS37-T | 1 OR 2 |
| 45 | PS21-I, PS36-C, PS37-T | 1 OR 2 |
| 46 | PS22-C, PS36-C, PS37-T | 1 OR 2 |
| 47 | PS23-C, PS36-C, PS37-T | 1 OR 2 |

TABLE A-2
Markers in ATP5G3 Gene Associated
with Better Antidepressant Response

| | | Copy |
|---|---|---|
| Marker | Polymorphisms | Number |
| 1 | PS1-T, PS2-C | 0 OR 1 |
| 2 | PS1-C | 1 OR 2 |

TABLE A-3
Markers in BCL2L1 Gene Associated with
Better Antidepressant Response

| | | Copy |
|---|---|---|
| Marker | Polymorphisms | Number |
| 1 | PS1-A | 1 OR 2 |
| 2 | PS1-G | 0 |
| 3 | PS2-C | 2 |
| 4 | PS3-G | 0 |
| 5 | PS2-C, PS4-T | 1 OR 2 |
| 6 | PS4-T, PS8-T | 1 OR 2 |
| 7 | PS4-T, PS5-G | 1 OR 2 |
| 8 | PS4-T, PS7-G | 1 OR 2 |
| 9 | PS6-C | 1 OR 2 |
| 10 | PS6-G | 1 OR 2 |
| 11 | PS8-T | 2 |
| 12 | PS3-A | 2 |
| 13 | PS5-G | 2 |
| 14 | PS7-G | 2 |
| 15 | PS1-G, PS4-T | 1 OR 2 |
| 16 | PS8-C | 1 OR 2 |
| 17 | PS2-T | 1 OR 2 |
| 18 | PS4-T, PS6-G | 1 OR 2 |
| 19 | PS5-C | 1 OR 2 |
| 20 | PS7-T | 1 OR 2 |

TABLE A-4
Markers in CYP2C9 Gene Associated with
Better Antidepressant Response

| | | Copy |
|---|---|---|
| Marker | Polymorphisms | Number |
| 1 | PS1-T, PS2-T | 2 |

TABLE A-5
Markers in DRD3 Gene Associated with
Better Antidepressant Response

| | | Copy |
|---|---|---|
| Marker | Polymorphisms | Number |
| 1 | PS1-C, PS7-G, PS10-A | 0 |
| 2 | PS1-C, PS9-G | 0 |
| 3 | PS1-C, PS2-G, PS6-T | 0 |
| 4 | PS1-C, PS2-G, PS7-G | 0 |
| 5 | PS1-C, PS3-G, PS10-A | 0 |
| 6 | PS1-C, PS4-A, PS10-A | 0 |
| 7 | PS1-C, PS6-T, PS10-A | 1 OR 2 |
| 8 | PS1-C, PS8-T, PS10-A | 1 OR 2 |
| 9 | PS1-C, PS2-G, PS10-T | 1 OR 2 |
| 10 | PS1-C, PS5-T, PS10-A | 1 OR 2 |

TABLE A-6
Markers in FOS Gene Associated with
Better Antidepressant Response

| | | Copy |
|---|---|---|
| Marker | Polymorphisms | Number |
| 1 | PS2-T, PS4-G, PS5-T | 1 OR 2 |
| 2 | PS2-T, PS5-T | 1 OR 2 |
| 3 | PS1-C, PS2-T | 0 OR 1 |
| 4 | PS1-C, PS2-T, PS3-G | 0 OR 1 |
| 5 | PS1-C, PS2-T, PS6-T | 0 OR 1 |

TABLE A-7
Markers in DTNBP1 Gene Associated
with Better Antidepressant Response

| | | Copy |
|---|---|---|
| Marker | Polymorphisms | Number |
| 1 | PS5-T, PS14-G, PS20-G | 0 |
| 2 | PS12-A, PS14-G, PS20-G | 0 |
| 3 | PS14-G, PS16-G, PS20-G | 0 |
| 4 | PS14-G, PS17-T, PS20-G | 0 |
| 5 | PS14-G, PS18-G, PS20-G | 0 |
| 6 | PS14-G, PS19-G, PS20-G | 0 |
| 7 | PS14-G, PS20-G, PS21-A | 0 |
| 8 | PS22-G, PS23-G, PS24-C | 0 OR 1 |
| 9 | PS22-G, PS24-C | 0 OR 1 |
| 10 | PS1-T, PS14-G, PS20-G | 0 |
| 11 | PS2-C, PS6-G, PS14-G | 0 |
| 12 | PS1-T, PS14-G, PS20-G | 1 OR 2 |
| 13 | PS2-C, PS14-G, PS20-G | 1 OR 2 |
| 14 | PS3-C, PS14-G, PS20-G | 1 OR 2 |
| 15 | PS4-G, PS14-G, PS20-G | 1 OR 2 |
| 16 | PS7-G, PS14-G, PS20-G | 1 OR 2 |
| 17 | PS8-G, PS14-G, PS20-G | 1 OR 2 |
| 18 | PS10-G, PS14-G, PS20-G | 1 OR 2 |
| 19 | PS11-C, PS14-G, PS20-G | 1 OR 2 |
| 20 | PS13-A, PS14-G, PS20-G | 1 OR 2 |
| 21 | PS14-G, PS15-C, PS20-G | 1 OR 2 |
| 22 | PS2-C, PS9-C, PS14-G | 1 OR 2 |
| 23 | PS6-G, PS7-G, PS14-G | 1 OR 2 |
| 24 | PS6-G, PS8-G, PS14-G | 1 OR 2 |
| 25 | PS6-G, PS10-G, PS14-G | 1 OR 2 |
| 26 | PS6-G, PS11-C, PS14-G | 1 OR 2 |
| 27 | PS6-G, PS13-A, PS14-G | 1 OR 2 |
| 28 | PS6-G, PS14-G, PS15-C | 1 OR 2 |
| 29 | PS6-G, PS14-G, PS16-G | 1 OR 2 |
| 30 | PS7-G, PS9-C, PS14-G | 1 OR 2 |
| 31 | PS8-G, PS9-C, PS14-G | 1 OR 2 |
| 32 | PS9-C, PS10-G, PS14-G | 1 OR 2 |
| 33 | PS9-C, PS11-C, PS14-G | 1 OR 2 |
| 34 | PS9-C, PS13-A, PS14-G | 1 OR 2 |
| 35 | PS9-C, PS14-G, PS15-C | 1 OR 2 |
| 36 | PS9-C, PS14-G, PS16-G | 1 OR 2 |

TABLE A-8
Markers in GABRG3 Gene Associated
with Better Antidepressant Response

| | | Copy |
|---|---|---|
| Marker | Polymorphisms | Number |
| 1 | PS1-G, PS8-T, PS13-G | 2 |
| 2 | PS7-C, PS8-T | 2 |
| 3 | PS7-C, PS9-G | 2 |
| 4 | PS7-C, PS12-C | 2 |
| 5 | PS7-T | 0 |
| 6 | PS10-C | 0 |
| 7 | PS6-C | 0 |
| 8 | PS6-C, PS11-C | 0 |
| 9 | PS6-G, PS12-C | 2 |
| 10 | PS2-C, PS8-T, PS13-G | 2 |
| 11 | PS3-T, PS8-T, PS13-G | 2 |
| 12 | PS4-A, PS8-T, PS13-G | 2 |
| 13 | PS5-T, PS8-T, PS13-G | 2 |

TABLE A-9
Markers in GRIA4 Gene Associated with
Better Antidepressant Response

| | | Copy |
|---|---|---|
| Marker | Polymorphisms | Number |
| 1 | PS10-C, PS30-G | 0 |
| 2 | PS14-A, PS15-T, PS29-A | 1 OR 2 |
| 3 | PS6-C, PS14-A, PS15-T | 1 OR 2 |
| 4 | PS6-C, PS15-T, PS25-T | 1 OR 2 |
| 5 | PS1-A, PS17-A | 0 OR 1 |
| 6 | PS1-A, PS26-A, PS36-T | 0 |
| 7 | PS1-A, PS36-T, PS37-C | 0 |
| 8 | PS1-A, PS6-T, PS30-G | 0 |
| 9 | PS1-A, PS6-T, PS36-T | 0 |
| 10 | PS1-G | 1 OR 2 |
| 11 | PS1-G, PS6-C | 1 OR 2 |
| 12 | PS15-C | 0 |
| 13 | PS15-T, PS31-G | 2 |
| 14 | PS2-C, PS26-G | 1 OR 2 |
| 15 | PS2-C, PS29-A, PS38-T | 1 OR 2 |
| 16 | PS2-C, PS5-C, PS6-C | 1 OR 2 |
| 17 | PS2-C, PS6-C, PS11-C | 1 OR 2 |
| 18 | PS2-C, PS6-C, PS15-T | 1 OR 2 |
| 19 | PS2-C, PS6-C, PS22-C | 1 OR 2 |
| 20 | PS2-C, PS6-C, PS26-G | 1 OR 2 |
| 21 | PS2-C, PS6-C, PS27-C | 1 OR 2 |
| 22 | PS2-C, PS6-C, PS38-T | 1 OR 2 |
| 23 | PS2-C, PS7-A, PS22-C | 1 OR 2 |
| 24 | PS2-C, PS8-G, PS26-G | 1 OR 2 |
| 25 | PS2-C, PS8-G, PS27-C | 1 OR 2 |
| 26 | PS24-C, PS26-A, PS36-T | 0 |
| 27 | PS25-T, PS31-G, PS32-A | 2 |
| 28 | PS26-A, PS28-A, PS36-T | 0 |
| 29 | PS26-A, PS36-T | 0 |
| 30 | PS26-G | 1 OR 2 |
| 31 | PS27-C, PS31-G | 2 |
| 32 | PS3-C, PS17-A | 0 OR 1 |
| 33 | PS3-C, PS36-T | 0 OR 1 |
| 34 | PS3-G | 1 OR 2 |
| 35 | PS3-G, PS11-C | 1 OR 2 |
| 36 | PS3-G, PS14-A | 1 OR 2 |
| 37 | PS3-G, PS22-C | 1 OR 2 |
| 38 | PS3-G, PS22-C, PS35-C | 1 OR 2 |
| 39 | PS3-G, PS5-C | 1 OR 2 |
| 40 | PS3-G, PS5-C, PS6-C | 1 OR 2 |
| 41 | PS3-G, PS6-C | 1 OR 2 |
| 42 | PS3-G, PS6-C, PS14-A | 1 OR 2 |
| 43 | PS31-C | 0 |
| 44 | PS31-G, PS32-A | 2 |
| 45 | PS31-G, PS33-T | 2 |
| 46 | PS31-G, PS38-T | 2 |
| 47 | PS32-G | 0 |
| 48 | PS36-T, PS37-C | 0 |
| 49 | PS5-C, PS6-C, PS16-T | 1 OR 2 |
| 50 | PS5-C, PS6-C, PS22-C | 1 OR 2 |
| 51 | PS6-C, PS10-T, PS22-C | 1 OR 2 |
| 52 | PS6-C, PS16-T, PS18-G | 1 OR 2 |
| 53 | PS6-C, PS16-T, PS27-C | 1 OR 2 |
| 54 | PS6-C, PS16-T, PS32-A | 1 OR 2 |
| 55 | PS6-C, PS16-T, PS38-T | 1 OR 2 |
| 56 | PS6-C, PS22-C | 1 OR 2 |
| 57 | PS6-C, PS22-C, PS24-C | 1 OR 2 |
| 58 | PS6-C, PS22-C, PS29-A | 1 OR 2 |
| 59 | PS6-C, PS22-C, PS35-C | 1 OR 2 |
| 60 | PS6-C, PS33-T | 1 OR 2 |
| 61 | PS6-C, PS7-A, PS16-T | 1 OR 2 |
| 62 | PS6-C, PS8-G, PS26-G | 1 OR 2 |
| 63 | PS6-C, PS8-G, PS27-C | 1 OR 2 |
| 64 | PS7-A, PS10-T, PS22-C | 1 OR 2 |
| 65 | PS7-A, PS16-T | 1 OR 2 |
| 66 | PS7-A, PS22-C | 1 OR 2 |
| 67 | PS7-A, PS22-C, PS35-C | 1 OR 2 |
| 68 | PS7-T, PS26-A, PS36-T | 0 |
| 69 | PS8-G, PS26-G | 1 OR 2 |
| 70 | PS8-G, PS27-C | 1 OR 2 |
| 71 | PS10-C, PS24-C, PS36-T | 1 OR 2 |
| 72 | PS10-C, PS27-C, PS36-T | 1 OR 2 |
| 73 | PS10-C, PS28-A, PS36-T | 1 OR 2 |
| 74 | PS10-C, PS29-T, PS36-T | 1 OR 2 |
| 75 | PS10-C, PS31-C | 1 OR 2 |
| 76 | PS10-C, PS33-T, PS36-T | 1 OR 2 |
| 77 | PS10-C, PS34-G, PS36-T | 1 OR 2 |
| 78 | PS15-T, PS21-G, PS29-A | 1 OR 2 |
| 79 | PS6-C, PS15-T, PS21-G | 1 OR 2 |
| 80 | PS2-C, PS13-T, PS27-C | 1 OR 2 |
| 81 | PS2-C, PS13-T, PS30-G | 1 OR 2 |
| 82 | PS2-C, PS13-T, PS33-T | 1 OR 2 |
| 83 | PS2-C, PS13-T, PS34-G | 1 OR 2 |
| 84 | PS2-C, PS13-T, PS38-T | 1 OR 2 |
| 85 | PS2-C, PS27-C, PS29-A | 1 OR 2 |
| 86 | PS2-C, PS29-A, PS30-G | 1 OR 2 |
| 87 | PS2-C, PS29-A, PS33-T | 1 OR 2 |
| 88 | PS2-C, PS29-A, PS34-G | 1 OR 2 |
| 89 | PS2-C, PS30-C, PS31-G | 1 OR 2 |
| 90 | PS2-C, PS37-T | 1 OR 2 |
| 91 | PS2-C, PS9-A, PS27-C | 1 OR 2 |
| 92 | PS2-C, PS9-A, PS30-G | 1 OR 2 |
| 93 | PS2-C, PS9-A, PS33-T | 1 OR 2 |
| 94 | PS2-C, PS9-A, PS34-G | 1 OR 2 |
| 95 | PS2-C, PS9-A, PS38-T | 1 OR 2 |
| 96 | PS4-C, PS13-T, PS27-C | 1 OR 2 |
| 97 | PS4-C, PS13-T, PS30-G | 1 OR 2 |
| 98 | PS4-C, PS13-T, PS33-T | 1 OR 2 |
| 99 | PS4-C, PS13-T, PS34-G | 1 OR 2 |
| 100 | PS4-C, PS13-T, PS38-T | 1 OR 2 |
| 101 | PS4-C, PS26-G | 1 OR 2 |
| 102 | PS4-C, PS27-C, PS29-A | 1 OR 2 |
| 103 | PS4-C, PS29-A, PS30-G | 1 OR 2 |
| 104 | PS4-C, PS29-A, PS33-T | 1 OR 2 |
| 105 | PS4-C, PS29-A, PS34-G | 1 OR 2 |
| 106 | PS4-C, PS30-G, PS31-G | 1 OR 2 |
| 107 | PS4-C, PS37-T | 1 OR 2 |
| 108 | PS4-C, PS9-A, PS27-C | 1 OR 2 |
| 109 | PS4-C, PS9-A, PS30-G | 1 OR 2 |
| 110 | PS4-C, PS9-A, PS33-T | 1 OR 2 |
| 111 | PS4-C, PS9-A, PS34-G | 1 OR 2 |
| 112 | PS4-C, PS9-A, PS38-T | 1 OR 2 |
| 113 | PS4-C, PS29-A, PS38-T | 1 OR 2 |
| 114 | PS4-C, PS5-C, PS6-C | 1 OR 2 |
| 115 | PS2-C, PS6-C, PS12-G | 1 OR 2 |
| 116 | PS4-C, PS6-C, PS11-C | 1 OR 2 |
| 117 | PS4-C, PS6-C, PS12-G | 1 OR 2 |
| 118 | PS4-C, PS6-C, PS15-T | 1 OR 2 |
| 119 | PS2-C, PS6-C, PS23-A | 1 OR 2 |
| 120 | PS4-C, PS6-C, PS22-C | 1 OR 2 |
| 121 | PS4-C, PS6-C, PS23-A | 1 OR 2 |
| 122 | PS2-C, PS6-C, PS37-T | 1 OR 2 |
| 123 | PS4-C, PS6-C, PS26-G | 1 OR 2 |
| 124 | PS4-C, PS6-C, PS37-T | 1 OR 2 |
| 125 | PS2-C, PS6-C, PS30-G | 1 OR 2 |
| 126 | PS2-C, PS6-C, PS33-T | 1 OR 2 |
| 127 | PS2-C, PS6-C, PS34-G | 1 OR 2 |
| 128 | PS4-C, PS6-C, PS27-C | 1 OR 2 |
| 129 | PS4-C, PS6-C, PS30-G | 1 OR 2 |
| 130 | PS4-C, PS6-C, PS33-T | 1 OR 2 |
| 131 | PS4-C, PS6-C, PS34-G | 1 OR 2 |
| 132 | PS4-C, PS6-C, PS38-T | 1 OR 2 |
| 133 | PS2-C, PS7-A, PS18-G | 1 OR 2 |
| 134 | PS2-C, PS7-A, PS23-A | 1 OR 2 |
| 135 | PS2-C, PS7-A, PS27-C | 1 OR 2 |
| 136 | PS2-C, PS7-A, PS30-G | 1 OR 2 |
| 137 | PS2-C, PS7-A, PS32-A | 1 OR 2 |
| 138 | PS2-C, PS7-A, PS33-T | 1 OR 2 |
| 139 | PS2-C, PS7-A, PS34-G | 1 OR 2 |
| 140 | PS2-C, PS7-A, PS38-T | 1 OR 2 |
| 141 | PS4-C, PS7-A, PS18-G | 1 OR 2 |
| 142 | PS4-C, PS7-A, PS22-C | 1 OR 2 |
| 143 | PS4-C, PS7-A, PS23-A | 1 OR 2 |
| 144 | PS4-C, PS7-A, PS27-C | 1 OR 2 |
| 145 | PS4-C, PS7-A, PS30-G | 1 OR 2 |
| 146 | PS4-C, PS7-A, PS32-A | 1 OR 2 |
| 147 | PS4-C, PS7-A, PS33-T | 1 OR 2 |
| 148 | PS4-C, PS7-A, PS34-G | 1 OR 2 |
| 149 | PS4-C, PS7-A, PS38-T | 1 OR 2 |
| 150 | PS2-C, PS8-G, PS37-T | 1 OR 2 |
| 151 | PS4-C, PS8-G, PS26-G | 1 OR 2 |
| 152 | PS4-C, PS8-G, PS37-T | 1 OR 2 |
| 153 | PS2-C, PS8-G, PS30-G | 1 OR 2 |
| 154 | PS2-C, PS8-G, PS33-T | 1 OR 2 |
| 155 | PS2-C, PS8-G, PS34-G | 1 OR 2 |
| 156 | PS2-C, PS8-G, PS38-T | 1 OR 2 |
| 157 | PS4-C, PS8-G, PS27-C | 1 OR 2 |
| 158 | PS4-C, PS8-G, PS30-G | 1 OR 2 |
| 159 | PS4-C, PS8-G, PS33-T | 1 OR 2 |
| 160 | PS4-C, PS8-G, PS34-G | 1 OR 2 |
| 161 | PS4-C, PS8-G, PS38-T | 1 OR 2 |
| 162 | PS24-C, PS36-T, PS37-C | 1 OR 2 |
| 163 | PS28-A, PS36-T, PS37-C | 1 OR 2 |
| 164 | PS10-T, PS27-C, PS31-G | 1 OR 2 |
| 165 | PS10-T, PS31-G, PS33-T | 1 OR 2 |
| 166 | PS10-T, PS31-G, PS34-G | 1 OR 2 |
| 167 | PS24-C, PS29-A, PS38-T | 1 OR 2 |
| 168 | PS24-C, PS37-T | 1 OR 2 |
| 169 | PS27-C, PS29-A | 1 OR 2 |
| 170 | PS27-C, PS31-G, PS36-T | 1 OR 2 |
| 171 | PS27-C, PS37-T | 1 OR 2 |
| 172 | PS28-A, PS29-A, PS38-T | 1 OR 2 |
| 173 | PS28-A, PS37-T | 1 OR 2 |
| 174 | PS29-A, PS30-G | 1 OR 2 |
| 175 | PS29-A, PS33-T | 1 OR 2 |
| 176 | PS29-A, PS34-G | 1 OR 2 |
| 177 | PS30-G, PS31-G | 1 OR 2 |
| 178 | PS30-G, PS37-T | 1 OR 2 |
| 179 | PS31-G, PS33-T, PS36-T | 1 OR 2 |
| 180 | PS31-G, PS34-G, PS36-T | 1 OR 2 |
| 181 | PS33-T, PS37-T | 1 OR 2 |
| 182 | PS34-G, PS37-T | 1 OR 2 |
| 183 | PS28-A, PS31-G, PS38-T | 2 |
| 184 | PS31-G, PS34-G | 2 |
| 185 | PS3-G, PS12-G | 1 OR 2 |
| 186 | PS3-G, PS18-G | 1 OR 2 |
| 187 | PS3-G, PS23-A | 1 OR 2 |
| 188 | PS3-G, PS26-G | 1 OR 2 |
| 189 | PS3-G, PS27-C | 1 OR 2 |
| 190 | PS3-G, PS30-G | 1 OR 2 |
| 191 | PS3-G, PS32-A | 1 OR 2 |
| 192 | PS3-G, PS33-T | 1 OR 2 |
| 193 | PS3-G, PS34-G | 1 OR 2 |
| 194 | PS3-G, PS37-T | 1 OR 2 |
| 195 | PS3-G, PS38-T | 1 OR 2 |
| 196 | PS3-G, PS7-A | 1 OR 2 |
| 197 | PS3-G, PS6-C, PS7-A | 1 OR 2 |
| 198 | PS13-C, PS16-T, PS30-G | 1 OR 2 |
| 199 | PS13-C, PS19-T, PS30-G | 1 OR 2 |
| 200 | PS13-C, PS20-T, PS30-G | 1 OR 2 |
| 201 | PS13-C, PS22-C, PS30-G | 1 OR 2 |
| 202 | PS13-C, PS23-A, PS30-G | 1 OR 2 |
| 203 | PS16-T, PS26-A, PS30-G | 1 OR 2 |
| 204 | PS19-T, PS26-A, PS30-G | 1 OR 2 |
| 205 | PS20-T, PS26-A, PS30-G | 1 OR 2 |
| 206 | PS22-C, PS26-A, PS30-G | 1 OR 2 |
| 207 | PS23-A, PS26-A, PS30-G | 1 OR 2 |
| 208 | PS24-C, PS26-A, PS30-G | 1 OR 2 |
| 209 | PS26-A, PS30-G, PS33-T | 1 OR 2 |
| 210 | PS26-A, PS33-T, PS36-T | 1 OR 2 |
| 211 | PS27-C, PS36-T, PS37-C | 1 OR 2 |
| 212 | PS29-T, PS30-G | 1 OR 2 |
| 213 | PS29-T, PS36-T | 1 OR 2 |
| 214 | PS30-G, PS37-C | 1 OR 2 |
| 215 | PS33-T, PS36-T, PS37-C | 1 OR 2 |
| 216 | PS34-G, PS36-T, PS37-C | 1 OR 2 |
| 217 | PS9-G, PS16-T, PS30-G | 1 OR 2 |
| 218 | PS9-G, PS19-T, PS30-G | 1 OR 2 |
| 219 | PS9-G, PS20-T, PS30-G | 1 OR 2 |
| 220 | PS9-G, PS22-C, PS30-G | 1 OR 2 |
| 221 | PS9-G, PS23-A, PS30-G | 1 OR 2 |
| 222 | PS5-C, PS6-C, PS19-T | 1 OR 2 |
| 223 | PS5-C, PS6-C, PS20-T | 1 OR 2 |
| 224 | PS5-C, PS6-C, PS23-A | 1 OR 2 |
| 225 | PS6-C, PS10-T, PS23-A | 1 OR 2 |
| 226 | PS6-C, PS18-G, PS19-T | 1 OR 2 |
| 227 | PS6-C, PS18-G, PS20-T | 1 OR 2 |
| 228 | PS6-C, PS16-T, PS30-G | 1 OR 2 |
| 229 | PS6-C, PS16-T, PS33-T | 1 OR 2 |
| 230 | PS6-C, PS16-T, PS34-G | 1 OR 2 |
| 231 | PS6-C, PS19-T, PS27-C | 1 OR 2 |
| 232 | PS6-C, PS19-T, PS30-G | 1 OR 2 |
| 233 | PS6-C, PS19-T, PS33-T | 1 OR 2 |
| 234 | PS6-C, PS19-T, PS34-G | 1 OR 2 |
| 235 | PS6-C, PS20-T, PS27-C | 1 OR 2 |
| 236 | PS6-C, PS20-T, PS30-G | 1 OR 2 |
| 237 | PS6-C, PS20-T, PS33-T | 1 OR 2 |
| 238 | PS6-C, PS20-T, PS34-G | 1 OR 2 |
| 239 | PS6-C, PS19-T, PS32-A | 1 OR 2 |
| 240 | PS6-C, PS20-T, PS32-A | 1 OR 2 |
| 241 | PS6-C, PS19-T, PS38-T | 1 OR 2 |
| 242 | PS6-C, PS20-T, PS38-T | 1 OR 2 |
| 243 | PS6-C, PS23-A | 1 OR 2 |
| 244 | PS6-C, PS22-C, PS27-C | 1 OR 2 |
| 245 | PS6-C, PS22-C, PS28-A | 1 OR 2 |
| 246 | PS6-C, PS22-C, PS30-G | 1 OR 2 |
| 247 | PS6-C, PS22-C, PS33-T | 1 OR 2 |
| 248 | PS6-C, PS22-C, PS34-G | 1 OR 2 |
| 249 | PS6-C, PS23-A, PS24-C | 1 OR 2 |
| 250 | PS6-C, PS23-A, PS27-C | 1 OR 2 |
| 251 | PS6-C, PS23-A, PS28-A | 1 OR 2 |
| 252 | PS6-C, PS23-A, PS30-G | 1 OR 2 |
| 253 | PS6-C, PS23-A, PS33-T | 1 OR 2 |
| 254 | PS6-C, PS23-A, PS34-G | 1 OR 2 |
| 255 | PS6-C, PS22-C, PS31-G | 1 OR 2 |
| 256 | PS6-C, PS23-A, PS29-A | 1 OR 2 |
| 257 | PS6-C, PS23-A, PS31-G | 1 OR 2 |
| 258 | PS6-C, PS24-C, PS38-T | 1 OR 2 |
| 259 | PS6-C, PS27-C | 1 OR 2 |
| 260 | PS6-C, PS28-A, PS38-T | 1 OR 2 |
| 261 | PS6-C, PS30-G | 1 OR 2 |
| 262 | PS6-C, PS34-G | 1 OR 2 |
| 263 | PS6-C, PS7-A, PS19-T | 1 OR 2 |
| 264 | PS6-C, PS7-A, PS20-T | 1 OR 2 |
| 265 | PS6-C, PS13-T, PS26-G | 1 OR 2 |
| 266 | PS6-C, PS13-T, PS27-C | 1 OR 2 |
| 267 | PS6-C, PS13-T, PS30-G | 1 OR 2 |
| 268 | PS6-C, PS13-T, PS33-T | 1 OR 2 |
| 269 | PS6-C, PS13-T, PS34-G | 1 OR 2 |
| 270 | PS6-C, PS13-T, PS37-T | 1 OR 2 |
| 271 | PS6-C, PS13-T, PS38-T | 1 OR 2 |
| 272 | PS6-C, PS16-T, PS26-G | 1 OR 2 |
| 273 | PS6-C, PS16-T, PS37-T | 1 OR 2 |
| 274 | PS6-C, PS19-T, PS26-G | 1 OR 2 |
| 275 | PS6-C, PS19-T, PS37-T | 1 OR 2 |
| 276 | PS6-C, PS20-T, PS26-G | 1 OR 2 |
| 277 | PS6-C, PS20-T, PS37-T | 1 OR 2 |
| 278 | PS6-C, PS8-G, PS37-T | 1 OR 2 |
| 279 | PS6-C, PS9-A, PS26-G | 1 OR 2 |
| 280 | PS6-C, PS9-A, PS27-C | 1 OR 2 |
| 281 | PS6-C, PS9-A, PS30-G | 1 OR 2 |
| 282 | PS6-C, PS9-A, PS33-T | 1 OR 2 |
| 283 | PS6-C, PS9-A, PS34-G | 1 OR 2 |
| 284 | PS6-C, PS9-A, PS37-T | 1 OR 2 |
| 285 | PS6-C, PS9-A, PS38-T | 1 OR 2 |
| 286 | PS6-C, PS8-G, PS30-G | 1 OR 2 |
| 287 | PS6-C, PS8-G, PS33-T | 1 OR 2 |
| 288 | PS6-C, PS8-G, PS34-G | 1 OR 2 |
| 289 | PS6-C, PS8-G, PS38-T | 1 OR 2 |
| 290 | PS7-A, PS10-T, PS23-A | 1 OR 2 |
| 291 | PS7-A, PS19-T | 1 OR 2 |
| 292 | PS7-A, PS20-T | 1 OR 2 |
| 293 | PS7-A, PS23-A | 1 OR 2 |
| 294 | PS7-T, PS36-T, PS37-C | 1 OR 2 |
| 295 | PS8-G, PS10-T, PS27-C | 1 OR 2 |
| 296 | PS8-G, PS10-T, PS30-G | 1 OR 2 |
| 297 | PS8-G, PS10-T, PS33-T | 1 OR 2 |
| 298 | PS8-G, PS10-T, PS34-G | 1 OR 2 |
| 299 | PS8-G, PS10-T, PS38-T | 1 OR 2 |
| 300 | PS8-G, PS13-T, PS27-C | 1 OR 2 |
| 301 | PS8-G, PS13-T, PS30-G | 1 OR 2 |
| 302 | PS8-G, PS13-T, PS33-T | 1 OR 2 |
| 303 | PS8-G, PS13-T, PS34-G | 1 OR 2 |
| 304 | PS8-G, PS13-T, PS38-T | 1 OR 2 |
| 305 | PS8-G, PS27-C, PS29-A | 1 OR 2 |
| 306 | PS8-G, PS27-C, PS31-G | 1 OR 2 |
| 307 | PS8-G, PS29-A, PS30-G | 1 OR 2 |
| 308 | PS8-G, PS29-A, PS33-T | 1 OR 2 |
| 309 | PS8-G, PS29-A, PS34-G | 1 OR 2 |
| 310 | PS8-G, PS29-A, PS38-T | 1 OR 2 |
| 311 | PS8-G, PS30-G, PS31-G | 1 OR 2 |
| 312 | PS8-G, PS31-G, PS33-T | 1 OR 2 |
| 313 | PS8-G, PS31-G, PS34-G | 1 OR 2 |
| 314 | PS8-G, PS31-G, PS38-T | 1 OR 2 |
| 315 | PS8-G, PS37-T | 1 OR 2 |
| 316 | PS8-G, PS9-A, PS27-C | 1 OR 2 |
| 317 | PS8-G, PS9-A, PS30-G | 1 OR 2 |
| 318 | PS8-G, PS9-A, PS33-T | 1 OR 2 |
| 319 | PS8-G, PS9-A, PS34-G | 1 OR 2 |
| 320 | PS8-G, PS9-A, PS38-T | 1 OR 2 |
| 321 | PS8-G, PS30-G | 1 OR 2 |
| 322 | PS8-G, PS33-T | 1 OR 2 |
| 323 | PS8-G, PS34-G | 1 OR 2 |
| 324 | PS8-G, PS38-T | 1 OR 2 |

TABLE A-10
Markers in LAMA4 Gene Associated
with Better Antidepressant Response

| | | Copy |
|---|---|---|
| Marker | Polymorphisms | Number |
| 1 | PS8-G | 1 OR 2 |
| 2 | PS3-T, PS8-G | 1 OR 2 |
| 3 | PS4-T, PS7-A, PS11-T | 0 OR 1 |
| 4 | PS4-T, PS10-A, PS11-T | 0 OR 1 |
| 5 | PS4-T, PS11-T | 0 OR 1 |
| 6 | PS5-G, PS9-A | 1 OR 2 |
| 7 | PS6-G, PS9-A | 1 OR 2 |
| 8 | PS9-A | 1 OR 2 |
| 9 | PS11-C | 1 OR 2 |
| 10 | PS1-A, PS5-G, PS9-A | 1 OR 2 |
| 11 | PS2-G, PS9-T | 0 OR 1 |

TABLE A-11
Markers in MAPK1 Gene Associated
with Better Antidepressant Response

| | | Copy |
|---|---|---|
| Marker | Polymorphisms | Number |
| 1 | PS1-G | 1 OR 2 |
| 2 | PS2-G | 1 OR 2 |
| 3 | PS2-G, PS5-G | 1 OR 2 |
| 4 | PS13-C | 1 OR 2 |
| 5 | PS7-G | 1 OR 2 |
| 6 | PS3-G, PS14-T | 1 OR 2 |
| 7 | PS4-G, PS13-C | 1 OR 2 |
| 8 | PS4-G, PS7-G | 1 OR 2 |
| 9 | PS1-G, PS12-A | 1 OR 2 |
| 10 | PS1-G, PS13-C | 1 OR 2 |
| 11 | PS1-G, PS5-G | 1 OR 2 |
| 12 | PS8-G, PS13-C | 1 OR 2 |
| 13 | PS9-C, PS13-C | 1 OR 2 |
| 14 | PS10-G, PS13-C | 1 OR 2 |
| 15 | PS11-C, PS13-C | 1 OR 2 |
| 16 | PS2-G, PS13-C | 1 OR 2 |
| 17 | PS3-G, PS13-C | 1 OR 2 |
| 18 | PS6-T, PS13-C | 1 OR 2 |
| 19 | PS1-G, PS4-G, PS12-A | 1 OR 2 |
| 20 | PS1-G, PS4-G, PS13-C | 1 OR 2 |
| 21 | PS1-G, PS4-G, PS5-G | 1 OR 2 |
| 22 | PS2-G, PS4-G, PS13-C | 1 OR 2 |
| 23 | PS3-G, PS4-G, PS13-C | 1 OR 2 |
| 24 | PS4-G, PS8-G, PS13-C | 1 OR 2 |
| 25 | PS4-G, PS9-C, PS13-C | 1 OR 2 |
| 26 | PS4-G, PS10-G, PS13-C | 1 OR 2 |
| 27 | PS4-G, PS11-C, PS13-C | 1 OR 2 |
| 28 | PS4-G, PS6-T, PS13-C | 1 OR 2 |

TABLE A-12
Markers in NPY1R Gene Associated with
Better Antidepressant Response

| | | Copy |
|---|---|---|
| Marker | Polymorphisms | Number |
| 1 | PS1-C, PS3-T, PS8-A | 1 OR 2 |
| 2 | PS1-C, PS3-T, PS7-T | 1 OR 2 |
| 3 | PS2-G, PS3-T, PS8-T | 0 OR 1 |
| 4 | PS3-T, PS8-A | 1 OR 2 |
| 5 | PS3-T, PS4-A, PS7-T | 1 OR 2 |
| 6 | PS3-T, PS5-T, PS7-T | 1 OR 2 |
| 7 | PS3-T, PS6-G, PS7-T | 1 OR 2 |
| 8 | PS3-T, PS7-T | 1 OR 2 |
| 9 | PS4-A, PS7-T | 1 OR 2 |
| 10 | PS7-T | 1 OR 2 |
| 11 | PS1-C, PS7-T | 1 OR 2 |
| 12 | PS8-A | 1 OR 2 |
| 13 | PS2-G, PS8-T | 0 OR 1 |
| 14 | PS5-T, PS7-T | 1 OR 2 |

TABLE A-13
Markers in OPRD1 Gene Associated with
Better Antidepressant Response

| | | Copy |
|---|---|---|
| Marker | Polymorphisms | Number |
| 1 | PS10-T, PS12-A, PS14-A | 2 |
| 2 | PS13-G, PS14-T | 0 |
| 3 | PS3-T, PS12-A, PS14-A | 2 |
| 4 | PS3-T, PS4-T, PS14-A | 2 |
| 5 | PS4-T, PS6-C, PS13-A | 2 |
| 6 | PS1-G, PS4-C, PS14-A | 0 |
| 7 | PS12-A, PS14-A | 2 |
| 8 | PS2-A, PS12-A, PS14-A | 2 |
| 9 | PS2-A, PS4-T, PS14-A | 2 |
| 10 | PS4-C, PS11-A, PS14-A | 0 |
| 11 | PS4-T, PS6-C, PS14-A | 2 |
| 12 | PS6-C, PS12-A, PS14-A | 2 |
| 13 | PS3-T, PS9-G, PS14-A | 2 |
| 14 | PS3-T, PS5-A, PS14-A | 2 |
| 15 | PS3-T, PS7-G, PS14-A | 2 |
| 16 | PS3-T, PS8-A, PS14-A | 2 |
| 17 | PS6-C, PS9-G, PS13-A | 2 |
| 18 | PS2-A, PS9-G, PS14-A | 2 |
| 19 | PS2-A, PS5-A, PS14-A | 2 |
| 20 | PS2-A, PS7-G, PS14-A | 2 |
| 21 | PS2-A, PS8-A, PS14-A | 2 |
| 22 | PS4-T, PS10-T, PS14-A | 2 |
| 23 | PS5-A, PS10-T, PS14-A | 2 |
| 24 | PS5-A, PS6-C, PS14-A | 2 |
| 25 | PS7-G, PS10-T, PS14-A | 2 |
| 26 | PS8-A, PS10-T, PS14-A | 2 |

TABLE A-14
Markers in OPRM1 Gene Associated
with Better Antidepressant Response

| | | Copy |
|---|---|---|
| Marker | Polymorphisms | Number |
| 1 | PS7-C, PS28-C | 0 |
| 2 | PS9-G, PS33-T, PS35-A | 1 OR 2 |
| 3 | PS12-C, PS31-G, PS35-A | 1 OR 2 |
| 4 | PS12-C, PS34-G, PS35-A | 1 OR 2 |
| 5 | PS14-A, PS31-G, PS35-A | 1 OR 2 |
| 6 | PS14-A, PS34-G, PS35-A | 1 OR 2 |
| 7 | PS20-T, PS34-G, PS35-A | 1 OR 2 |
| 8 | PS23-C, PS31-G, PS35-A | 1 OR 2 |
| 9 | PS23-C, PS34-G, PS35-A | 1 OR 2 |
| 10 | PS23-C, PS34-G, PS37-A | 1 OR 2 |
| 11 | PS23-C, PS34-G, PS41-A | 1 OR 2 |
| 12 | PS26-G, PS38-C | 0 OR 1 |
| 13 | PS31-G, PS34-G, PS35-A | 1 OR 2 |
| 14 | PS31-G, PS35-A | 1 OR 2 |
| 15 | PS34-G, PS35-A | 1 OR 2 |
| 16 | PS35-A, PS37-A, PS39-C | 1 OR 2 |
| 17 | PS38-A | 1 OR 2 |
| 18 | PS9-G, PS31-G, PS35-A | 1 OR 2 |
| 19 | PS14-A, PS33-T, PS35-A | 1 OR 2 |
| 20 | PS21-G, PS31-G, PS35-A | 1 OR 2 |
| 21 | PS24-G, PS38-C | 0 OR 1 |
| 22 | PS26-G, PS30-C | 0 OR 1 |
| 23 | PS29-A, PS30-C | 0 OR 1 |
| 24 | PS29-A, PS38-C | 0 OR 1 |
| 25 | PS31-G, PS35-A, PS40-G | 1 OR 2 |
| 26 | PS31-G, PS36-T, PS37-A | 1 OR 2 |
| 27 | PS31-G, PS36-T, PS40-G | 1 OR 2 |
| 28 | PS33-T, PS35-A | 1 OR 2 |
| 29 | PS33-T, PS36-T, PS40-G | 1 OR 2 |
| 30 | PS5-G, PS8-G, PS28-C | 1 OR 2 |
| 31 | PS5-G, PS16-A, PS28-C | 1 OR 2 |
| 32 | PS6-C, PS8-G, PS28-C | 1 OR 2 |
| 33 | PS6-C, PS16-A, PS28-C | 1 OR 2 |
| 34 | PS8-G, PS13-G, PS28-C | 1 OR 2 |
| 35 | PS11-G, PS28-C | 1 OR 2 |
| 36 | PS1-C, PS8-G, PS28-C | 1 OR 2 |
| 37 | PS1-C, PS9-G, PS28-C | 1 OR 2 |
| 38 | PS1-C, PS10-T, PS28-C | 1 OR 2 |
| 39 | PS1-C, PS12-C, PS28-C | 1 OR 2 |
| 40 | PS1-C, PS14-A, PS28-C | 1 OR 2 |
| 41 | PS1-C, PS15-G, PS28-C | 1 OR 2 |
| 42 | PS1-C, PS16-A, PS28-C | 1 OR 2 |
| 43 | PS1-C, PS17-C, PS28-C | 1 OR 2 |
| 44 | PS1-C, PS18-T, PS28-C | 1 OR 2 |
| 45 | PS1-C, PS19-A, PS28-C | 1 OR 2 |
| 46 | PS13-G, PS16-A, PS28-C | 1 OR 2 |
| 47 | PS2-A, PS8-G, PS28-C | 1 OR 2 |
| 48 | PS2-A, PS9-G, PS28-C | 1 OR 2 |
| 49 | PS2-A, PS10-T, PS28-C | 1 OR 2 |
| 50 | PS2-A, PS12-C, PS28-C | 1 OR 2 |
| 51 | PS2-A, PS14-A, PS28-C | 1 OR 2 |
| 52 | PS2-A, PS15-G, PS28-C | 1 OR 2 |
| 53 | PS2-A, PS16-A, PS28-C | 1 OR 2 |
| 54 | PS2-A, PS17-C, PS28-C | 1 OR 2 |
| 55 | PS2-A, PS18-T, PS28-C | 1 OR 2 |
| 56 | PS2-A, PS19-A, PS28-C | 1 OR 2 |
| 57 | PS3-T, PS8-G, PS28-C | 1 OR 2 |
| 58 | PS3-T, PS9-G, PS28-C | 1 OR 2 |
| 59 | PS3-T, PS10-T, PS28-C | 1 OR 2 |
| 60 | PS3-T, PS12-C, PS28-C | 1 OR 2 |
| 61 | PS3-T, PS14-A, PS28-C | 1 OR 2 |
| 62 | PS3-T, PS15-G, PS28-C | 1 OR 2 |
| 63 | PS3-T, PS16-A, PS28-C | 1 OR 2 |
| 64 | PS3-T, PS17-C, PS28-C | 1 OR 2 |
| 65 | PS3-T, PS18-T, PS28-C | 1 OR 2 |
| 66 | PS3-T, PS19-A, PS28-C | 1 OR 2 |
| 67 | PS4-C, PS8-G, PS28-C | 1 OR 2 |
| 68 | PS4-C, PS9-G, PS28-C | 1 OR 2 |
| 69 | PS4-C, PS10-T, PS28-C | 1 OR 2 |
| 70 | PS4-C, PS12-C, PS28-C | 1 OR 2 |
| 71 | PS4-C, PS14-A, PS28-C | 1 OR 2 |
| 72 | PS4-C, PS15-G, PS28-C | 1 OR 2 |
| 73 | PS4-C, PS16-A, PS28-C | 1 OR 2 |
| 74 | PS4-C, PS17-C, PS28-C | 1 OR 2 |
| 75 | PS4-C, PS18-T, PS28-C | 1 OR 2 |
| 76 | PS4-C, PS19-A, PS28-C | 1 OR 2 |
| 77 | PS10-T, PS33-T, PS35-A | 1 OR 2 |
| 78 | PS17-C, PS33-T, PS35-A | 1 OR 2 |
| 79 | PS9-G, PS31-G, PS35-A | 1 OR 2 |
| 80 | PS9-G, PS32-T, PS35-A | 1 OR 2 |
| 81 | PS10-T, PS31-G, PS35-A | 1 OR 2 |
| 82 | PS10-T, PS32-T, PS35-A | 1 OR 2 |
| 83 | PS12-C, PS32-T, PS35-A | 1 OR 2 |
| 84 | PS15-G, PS31-G, PS35-A | 1 OR 2 |
| 85 | PS15-G, PS32-T, PS35-A | 1 OR 2 |
| 86 | PS17-C, PS31-G, PS35-A | 1 OR 2 |
| 87 | PS17-C, PS32-T, PS35-A | 1 OR 2 |
| 88 | PS18-T, PS31-G, PS35-A | 1 OR 2 |
| 89 | PS18-T, PS32-T, PS35-A | 1 OR 2 |
| 90 | PS19-A, PS31-G, PS35-A | 1 OR 2 |
| 91 | PS19-A, PS32-T, PS35-A | 1 OR 2 |
| 92 | PS9-G, PS34-G, PS35-A | 1 OR 2 |
| 93 | PS10-T, PS34-G, PS35-A | 1 OR 2 |
| 94 | PS15-G, PS34-G, PS35-A | 1 OR 2 |
| 95 | PS17-C, PS34-G, PS35-A | 1 OR 2 |
| 96 | PS18-T, PS34-G, PS35-A | 1 OR 2 |
| 97 | PS19-A, PS34-G, PS35-A | 1 OR 2 |
| 98 | PS14-A, PS32-T, PS35-A | 1 OR 2 |
| 99 | PS25-T, PS34-G, PS35-A | 1 OR 2 |
| 100 | PS23-C, PS32-T, PS35-A | 1 OR 2 |
| 101 | PS32-T, PS34-G, PS35-A | 1 OR 2 |
| 102 | PS32-T, PS35-A | 1 OR 2 |
| 103 | PS21-G, PS32-T, PS35-A | 1 OR 2 |
| 104 | PS27-T, PS31-G, PS35-A | 1 OR 2 |
| 105 | PS27-T, PS32-T, PS35-A | 1 OR 2 |
| 106 | PS32-T, PS36-T, PS37-A | 1 OR 2 |
| 107 | PS32-T, PS36-T, PS40-G | 1 OR 2 |
| 108 | PS22-T, PS34-G, PS35-A | 1 OR 2 |

TABLE A-15
Markers in PER3 Gene Associated with
Better Antidepressant Response

| | | Copy |
|---|---|---|
| Marker | Polymorphisms | Number |
| 1 | PS1-C, PS12-T, PS24-4 | 0 |
| 2 | PS1-C, PS7-T, PS24-4 | 0 |
| 3 | PS1-T | 2 |
| 4 | PS9-C, PS12-T, PS24-4 | 0 |
| 5 | PS10-G, PS17-A | 2 |
| 6 | PS12-T, PS16-A, PS24-4 | 0 |
| 7 | PS12-T, PS24-4 | 0 |
| 8 | PS19-G | 0 |
| 9 | PS3-G, PS12-T, PS24-4 | 0 |
| 10 | PS6-C, PS10-G, PS14-G | 2 |
| 11 | PS6-C, PS10-G, PS17-A | 2 |
| 12 | PS1-C, PS12-T | 0 |
| 13 | PS1-C, PS3-G, PS12-T | 0 |
| 14 | PS10-G, PS13-T | 2 |
| 15 | PS10-G, PS17-A, PS21-G | 2 |
| 16 | PS12-T, PS19-G | 0 |
| 17 | PS12-T, PS19-G, PS22-G | 0 |
| 18 | PS12-T, PS22-G, PS24-4 | 0 |
| 19 | PS3-G, PS12-T, PS19-G | 0 |
| 20 | PS3-G, PS7-T, PS24-4 | 0 |
| 21 | PS7-T, PS9-C, PS24-4 | 0 |
| 22 | PS7-T, PS24-4 | 0 |
| 23 | PS1-C, PS18-C, PS24-4 | 1 OR 2 |
| 24 | PS1-C, PS24-4, PS27-A | 1 OR 2 |
| 25 | PS12-T, PS19-G, PS24-4 | 1 OR 2 |
| 26 | PS12-T, PS24-4, PS25-C | 1 OR 2 |
| 27 | PS18-C, PS19-G, PS24-4 | 1 OR 2 |
| 28 | PS18-C, PS24-4, PS25-C | 1 OR 2 |
| 29 | PS19-G, PS24-4, PS27-A | 1 OR 2 |
| 30 | PS24-4, PS25-C, PS27-A | 1 OR 2 |
| 31 | PS7-T, PS19-G, PS24-4 | 1 OR 2 |
| 32 | PS7-T, PS24-4, PS25-C | 1 OR 2 |
| 33 | PS10-G, PS12-T, PS17-A | 2 |
| 34 | PS10-G, PS17-A, PS18-C | 2 |
| 35 | PS10-G, PS17-A, PS27-A | 2 |
| 36 | PS12-T, PS13-T, PS29-G | 2 |
| 37 | PS12-T, PS15-C, PS17-A | 2 |
| 38 | PS12-T, PS17-A, PS29-G | 2 |
| 39 | PS13-T, PS18-C, PS29-G | 2 |
| 40 | PS13-T, PS27-A, PS29-G | 2 |
| 41 | PS15-C, PS17-A, PS18-C | 2 |
| 42 | PS15-C, PS17-A, PS27-A | 2 |
| 43 | PS2-A, PS12-T, PS29-G | 2 |
| 44 | PS2-A, PS18-C, PS29-G | 2 |
| 45 | PS2-A, PS27-A, PS29-G | 2 |
| 46 | PS2-A, PS7-T, PS29-G | 2 |
| 47 | PS2-A, PS8-T | 2 |
| 48 | PS17-A, PS18-C, PS29-G | 2 |
| 49 | PS17-A, PS27-A, PS29-G | 2 |
| 50 | PS19-C | 2 |
| 51 | PS25-G | 2 |
| 52 | PS4-C, PS12-T, PS29-G | 2 |
| 53 | PS4-C, PS18-C, PS29-G | 2 |
| 54 | PS4-C, PS27-A, PS29-G | 2 |
| 55 | PS4-C, PS7-T, PS29-G | 2 |
| 56 | PS4-C, PS8-T | 2 |
| 57 | PS5-C, PS12-T, PS29-G | 2 |
| 58 | PS5-C, PS18-C, PS29-G | 2 |
| 59 | PS5-C, PS27-A, PS29-G | 2 |
| 60 | PS5-C, PS7-T, PS29-G | 2 |
| 61 | PS5-C, PS8-T | 2 |
| 62 | PS7-T, PS13-T, PS29-G | 2 |
| 63 | PS7-T, PS17-A, PS29-G | 2 |
| 64 | PS8-T, PS11-C, PS14-G | 2 |
| 65 | PS8-T, PS11-C, PS20-C | 2 |
| 66 | PS8-T, PS11-C, PS28-G | 2 |
| 67 | PS8-T, PS13-T | 2 |
| 68 | PS8-T, PS17-A | 2 |
| 69 | PS9-C, PS18-C, PS24-4 | 1 OR 2 |
| 70 | PS9-C, PS24-4, PS27-A | 1 OR 2 |
| 71 | PS15-C, PS17-A | 2 |
| 72 | PS16-A, PS18-C, PS24-4 | 1 OR 2 |
| 73 | PS16-A, PS24-4, PS27-A | 1 OR 2 |
| 74 | PS18-C, PS24-4 | 1 OR 2 |
| 75 | PS24-4, PS27-A | 1 OR 2 |
| 76 | PS1-C | 1 OR 2 |
| 77 | PS25-C | 1 OR 2 |
| 78 | PS3-G, PS9-C, PS23-G | 1 OR 2 |
| 79 | PS3-G, PS9-C, PS26-T | 1 OR 2 |
| 80 | PS3-G, PS18-C, PS24-4 | 1 OR 2 |
| 81 | PS3-G, PS24-4, PS27-A | 1 OR 2 |
| 82 | PS6-C, PS14-G, PS15-C | 2 |
| 83 | PS1-C, PS18-C | 1 OR 2 |
| 84 | PS1-C, PS27-A | 1 OR 2 |
| 85 | PS10-G, PS11-C, PS20-C | 2 |
| 86 | PS10-G, PS11-C, PS28-G | 2 |
| 87 | PS11-C, PS15-C, PS20-C | 2 |
| 88 | PS11-C, PS15-C, PS28-G | 2 |
| 89 | PS13-T, PS15-C | 2 |
| 90 | PS18-C, PS19-G | 1 OR 2 |
| 91 | PS19-G, PS27-A | 1 OR 2 |
| 92 | PS3-G, PS18-C, PS19-G | 1 OR 2 |
| 93 | PS3-G, PS19-G, PS27-A | 1 OR 2 |

TABLE A-16
Markers in PLCB1 Gene Associated
with Better Antidepressant Response

| | | Copy |
|---|---|---|
| Marker | Polymorphisms | Number |
| 1 | PS1-A, PS14-A, PS15-A | 2 |
| 2 | PS1-A, PS15-A, PS17-T | 2 |
| 3 | PS1-A, PS5-A, PS14-A | 2 |
| 4 | PS1-A, PS5-A, PS17-T | 2 |
| 5 | PS14-A, PS15-A | 2 |
| 6 | PS14-A, PS15-A, PS16-T | 2 |
| 7 | PS15-A, PS16-T | 2 |
| 8 | PS15-A, PS17-T | 2 |
| 9 | PS2-A, PS13-C, PS14-A | 2 |
| 10 | PS2-A, PS13-C, PS16-T | 2 |
| 11 | PS2-A, PS13-C, PS17-T | 2 |
| 12 | PS2-A, PS5-A, PS14-A | 2 |
| 13 | PS2-A, PS5-A, PS16-T | 2 |
| 14 | PS2-A, PS5-A, PS17-T | 2 |
| 15 | PS3-G, PS14-A, PS15-A | 2 |
| 16 | PS3-G, PS5-A, PS14-A | 2 |
| 17 | PS4-G, PS14-A, PS15-A | 2 |
| 18 | PS4-G, PS15-A, PS17-T | 2 |
| 19 | PS5-A, PS14-A | 2 |
| 20 | PS5-A, PS14-A, PS16-T | 2 |
| 21 | PS5-A, PS16-T | 2 |
| 22 | PS5-A, PS17-T | 2 |
| 23 | PS1-A, PS13-C, PS14-A | 2 |
| 24 | PS1-A, PS13-C, PS17-T | 2 |
| 25 | PS1-A, PS9-G, PS14-A | 2 |
| 26 | PS1-A, PS10-A, PS14-A | 2 |
| 27 | PS1-A, PS11-A, PS14-A | 2 |
| 28 | PS1-A, PS12-G, PS14-A | 2 |
| 29 | PS1-A, PS6-G, PS14-A | 2 |
| 30 | PS1-A, PS7-G, PS14-A | 2 |
| 31 | PS1-A, PS8-C, PS14-A | 2 |
| 32 | PS4-G, PS9-G, PS14-A | 2 |
| 33 | PS4-G, PS10-A, PS14-A | 2 |
| 34 | PS4-G, PS11-A, PS14-A | 2 |
| 35 | PS4-G, PS12-G, PS14-A | 2 |
| 36 | PS4-G, PS5-A, PS14-A | 2 |
| 37 | PS4-G, PS6-G, PS14-A | 2 |
| 38 | PS4-G, PS7-G, PS14-A | 2 |
| 39 | PS4-G, PS8-C, PS14-A | 2 |
| 40 | PS1-A, PS9-G, PS17-T | 2 |
| 41 | PS1-A, PS10-A, PS17-T | 2 |
| 42 | PS1-A, PS11-A, PS17-T | 2 |
| 43 | PS1-A, PS12-G, PS17-T | 2 |
| 44 | PS1-A, PS6-G, PS17-T | 2 |
| 45 | PS1-A, PS7-G, PS17-T | 2 |
| 46 | PS1-A, PS8-C, PS17-T | 2 |
| 47 | PS4-G, PS9-G, PS17-T | 2 |
| 48 | PS4-G, PS10-A, PS17-T | 2 |
| 49 | PS4-G, PS11-A, PS17-T | 2 |
| 50 | PS4-G, PS12-G, PS17-T | 2 |
| 51 | PS4-G, PS5-A, PS17-T | 2 |
| 52 | PS4-G, PS6-G, PS17-T | 2 |
| 53 | PS4-G, PS7-G, PS17-T | 2 |
| 54 | PS4-G, PS8-C, PS17-T | 2 |
| 55 | PS13-C, PS14-A | 2 |
| 56 | PS13-C, PS14-A, PS16-T | 2 |
| 57 | PS13-C, PS17-T | 2 |
| 58 | PS13-C, PS16-T | 2 |
| 59 | PS2-A, PS14-A, PS15-A | 2 |
| 60 | PS2-A, PS15-A, PS16-T | 2 |
| 61 | PS2-A, PS15-A, PS17-T | 2 |
| 62 | PS2-A, PS9-G, PS14-A | 2 |
| 63 | PS2-A, PS10-A, PS14-A | 2 |
| 64 | PS2-A, PS11-A, PS14-A | 2 |
| 65 | PS2-A, PS12-G, PS14-A | 2 |
| 66 | PS2-A, PS6-G, PS14-A | 2 |
| 67 | PS2-A, PS7-G, PS14-A | 2 |
| 68 | PS2-A, PS8-C, PS14-A | 2 |
| 69 | PS2-A, PS9-G, PS16-T | 2 |
| 70 | PS2-A, PS10-A, PS16-T | 2 |
| 71 | PS2-A, PS11-A, PS16-T | 2 |
| 72 | PS2-A, PS12-G, PS16-T | 2 |
| 73 | PS2-A, PS6-G, PS16-T | 2 |
| 74 | PS2-A, PS7-G, PS16-T | 2 |
| 75 | PS2-A, PS8-C, PS16-T | 2 |
| 76 | PS2-A, PS9-G, PS17-T | 2 |
| 77 | PS2-A, PS10-A, PS17-T | 2 |
| 78 | PS2-A, PS11-A, PS17-T | 2 |
| 79 | PS2-A, PS12-G, PS17-T | 2 |
| 80 | PS2-A, PS6-G, PS17-T | 2 |
| 81 | PS2-A, PS7-G, PS17-T | 2 |
| 82 | PS2-A, PS8-C, PS17-T | 2 |
| 83 | PS3-G, PS13-C, PS14-A | 2 |
| 84 | PS3-G, PS13-C, PS17-T | 2 |
| 85 | PS3-G, PS15-A, PS17-T | 2 |
| 86 | PS3-G, PS9-G, PS14-A | 2 |
| 87 | PS3-G, PS9-G, PS17-T | 2 |
| 88 | PS3-G, PS10-A, PS14-A | 2 |
| 89 | PS3-G, PS10-A, PS17-T | 2 |
| 90 | PS3-G, PS11-A, PS14-A | 2 |
| 91 | PS3-G, PS11-A, PS17-T | 2 |
| 92 | PS3-G, PS12-G, PS14-A | 2 |
| 93 | PS3-G, PS12-G, PS17-T | 2 |
| 94 | PS3-G, PS5-A, PS17-T | 2 |
| 95 | PS3-G, PS6-G, PS14-A | 2 |
| 96 | PS3-G, PS6-G, PS17-T | 2 |
| 97 | PS3-G, PS7-G, PS14-A | 2 |
| 98 | PS3-G, PS7-G, PS17-T | 2 |
| 99 | PS3-G, PS8-C, PS14-A | 2 |
| 100 | PS3-G, PS8-C, PS17-T | 2 |
| 101 | PS4-G, PS13-C, PS14-A | 2 |
| 102 | PS4-G, PS13-C, PS17-T | 2 |
| 103 | PS9-G, PS14-A | 2 |
| 104 | PS10-A, PS14-A | 2 |
| 105 | PS11-A, PS14-A | 2 |
| 106 | PS12-G, PS14-A | 2 |
| 107 | PS6-G, PS14-A | 2 |
| 108 | PS7-G, PS14-A | 2 |
| 109 | PS8-C, PS14-A | 2 |
| 110 | PS9-G, PS14-A, PS16-T | 2 |
| 111 | PS9-G, PS17-T | 2 |
| 112 | PS10-A, PS14-A, PS16-T | 2 |
| 113 | PS10-A, PS17-T | 2 |
| 114 | PS11-A, PS14-A, PS16-T | 2 |
| 115 | PS11-A, PS17-T | 2 |
| 116 | PS12-G, PS14-A, PS16-T | 2 |
| 117 | PS12-G, PS17-T | 2 |
| 118 | PS6-G, PS14-A, PS16-T | 2 |
| 119 | PS6-G, PS17-T | 2 |
| 120 | PS7-G, PS14-A, PS16-T | 2 |
| 121 | PS7-G, PS17-T | 2 |
| 122 | PS8-C, PS14-A, PS16-T | 2 |
| 123 | PS8-C, PS17-T | 2 |
| 124 | PS9-G, PS16-T | 2 |
| 125 | PS10-A, PS16-T | 2 |
| 126 | PS11-A, PS16-T | 2 |
| 127 | PS12-G, PS16-T | 2 |
| 128 | PS6-G, PS16-T | 2 |
| 129 | PS7-G, PS16-T | 2 |
| 130 | PS8-C, PS16-T | 2 |

TABLE A-17
Markers in PSMD1 Associated with
Better Antidepressant Response

| | | Copy |
|---|---|---|
| Marker | Polymorphisms | Number |
| 1 | PS9-G | 1 OR 2 |
| 2 | PS1-C, PS5-G, PS7-G | 0 OR 1 |
| 3 | PS1-C, PS6-A, PS7-G | 0 OR 1 |
| 4 | PS1-C, PS7-G | 0 OR 1 |
| 5 | PS3-A, PS6-A, PS7-G | 0 OR 1 |
| 6 | PS3-A, PS7-G | 0 OR 1 |
| 7 | PS3-A, PS7-G, PS10-T | 0 OR 1 |
| 8 | PS4-A, PS7-A | 1 OR 2 |
| 9 | PS4-A, PS7-A, PS8-G | 1 OR 2 |
| 10 | PS5-G, PS7-G, PS10-T | 0 OR 1 |
| 11 | PS7-A | 1 OR 2 |
| 12 | PS7-A, PS8-G | 1 OR 2 |
| 13 | PS1-C, PS7-G | 2 |
| 14 | PS5-G, PS7-G | 2 |
| 15 | PS5-G, PS6-A, PS7-G | 2 |
| 16 | PS1-C, PS2-G, PS7-G | 2 |
| 17 | PS1-C, PS3-A, PS7-G | 2 |
| 18 | PS1-C, PS7-G, PS10-T | 2 |
| 19 | PS2-G, PS5-G, PS7-G | 2 |
| 20 | PS3-A, PS5-G, PS7-G | 2 |

TABLE A-18
Markers in ABI1 Gene Associated with
Better Antidepressant Response

| | | Copy |
|---|---|---|
| Marker | Polymorphisms | Number |
| 1 | PS1-A | 0 |
| 2 | PS2-T | 2 |
| 3 | PS3-A | 2 |

TABLE A-19
Markers in LOC402382 Gene Associated
with Better Antidepressant Response

| | | Copy |
|---|---|---|
| Marker | Polymorphisms | Number |
| 1 | PS1-T | 1 OR 2 |

TABLE A-20
Markers in NCALD Gene Associated with
Better Antidepressant Response

| | | Copy |
|---|---|---|
| Marker | Polymorphisms | Number |
| 1 | PS1-A | 2 |

TABLE A-21
Polymorphic Sites in the ACE Gene

| | | Position | | |
|---|---|---|---|---|
| PS | | in SEQ | Reference | Variant |
| Number | Poly ID | ID NO: 1 | Allele | Allele |
| 1 | 711180659 | 1327 | G | A |
| 2 | 711180681 | 1504 | G | C |
| 3 | 710922725 | 1633 | T | A |
| 4 | 710921732 | 1780 | C | T |
| 5 | 711088351 | 3105 | G | A |
| 6 | 710921315 | 3737 | C | G |
| 7 | 710920871 | 4455 | C | T |
| 8 | 710922167 | 4639 | C | T |
| 9 | 710923433 | 5261 | C | T |
| 10 | 710922265 | 5359 | C | T |
| 11 | 710920323 | 5378 | C | T |
| 12 | 710923078 | 5837 | G | A |
| 13 | 710920454 | 6390 | C | T |
| 14 | 710923435 | 6395 | G | T |
| 15 | 710921368 | 7258 | C | T |
| 16 | 710920726 | 7362 | C | T |
| 17 | 710920684 | 7488 | G | C |
| 18 | 710922605 | 11491 | A | G |
| 19 | 710922218 | 11720 | T | C |
| 20 | 710923183 | 11961 | T | C |
| 21 | 711205105 | 13330 | — | ATACAGT |
| | | | | CACTTTT |
| | | | | TTTTTTTT |
| | | | | TTTGAGA |
| | | | | CGGAGTC |
| | | | | TCGCTCT |
| | | | | GTCGCCC |
| | | | | AGGCTGG |
| | | | | AGTGCAG |
| | | | | TGGCGGG |
| | | | | ATCTCGG |
| | | | | CTCACTG |
| | | | | CAAGCTC |
| | | | | CGCCTCC |
| | | | | CGGGTTC |
| | | | | ACGCCAT |
| | | | | TCTCCTG |
| | | | | CCTCAGC |
| | | | | CTCCCAA |
| | | | | GTAGCTG |
| | | | | GGACCAC |
| | | | | AGGCGCC |
| | | | | CGCCACT |
| | | | | ACGCCCG |
| | | | | GCTAATT |
| | | | | TTTTGTA |
| | | | | TTTTTAG |
| | | | | TAGAGAC |
| | | | | GGGGTTT |
| | | | | CACCGTT |
| | | | | TTAGCCG |
| | | | | GGATGGT |
| | | | | CTCGATC |
| | | | | TCCTGAC |
| | | | | CTCGTGA |
| | | | | TCCGCCC |
| | | | | GCCTCGG |
| | | | | CCTCCCA |
| | | | | AAGTGCT |
| | | | | GGGATTA |
| | | | | CAGGCGTG |
| 22 | 711168334 | 13430 | G | C |
| 23 | 711168332 | 13438 | A | C |
| 24 | 711165639 | 16121 | G | A |
| 25 | 710921830 | 16245 | C | G |
| 26 | 710922422 | 18650 | C | T |
| 27 | 710923604 | 18892 | C | T |
| 28 | 710922379 | 18910 | C | T |
| 29 | 710921390 | 19070 | C | T |
| 30 | 710923235 | 19347 | G | A |
| 31 | 711168450 | 21791 | C | T |
| 32 | 711088695 | 25488 | T | C |
| 33 | 711088593 | 31196 | C | T |
| 34 | 711049196 | 32067 | T | C |
| 35 | 711049198 | 32160 | A | T |
| 36 | 711088813 | 36705 | T | C |
| 37 | 711088713 | 45558 | T | C |

TABLE A-22
Polymorphic Sites in the ATP5G3 Gene

| | | Position | | |
|---|---|---|---|---|
| PS | | in SEQ | Reference | Variant |
| Number | Poly ID | ID NO: 2 | Allele | Allele |
| 1 | 710908281 | 5810 | T | C |
| 2 | 711089030 | 10530 | C | T |

TABLE A-23
Polymorphic Sites in the BCL2L1 Gene

| | | Position | | |
|---|---|---|---|---|
| PS | | in SEQ | Reference | Variant |
| Number | Poly ID | ID NO: 3 | Allele | Allele |
| 1 | 710946639 | 273 | A | G |
| 2 | 710946636 | 12963 | C | T |
| 3 | 710946633 | 24774 | A | G |
| 4 | 710946624 | 31091 | T | C |
| 5 | 710946630 | 36225 | G | C |
| 6 | 710988411 | 45435 | C | G |
| 7 | 710946642 | 47751 | G | T |
| 8 | 710946645 | 47852 | T | C |

TABLE A-24
Polymorphic Sites in the CYP2C9 Gene

| | | Position | | |
|---|---|---|---|---|
| PS | | in SEQ | Reference | Variant |
| Number | Poly ID | ID NO: 4 | Allele | Allele |
| 1 | 13819187 | 312 | T | C |
| 2 | 710859989 | 2512 | T | C |

TABLE A-25
Polymorphic Sites in the DRD3 Gene

| | | Position | | |
|---|---|---|---|---|
| PS | | in SEQ | Reference | Variant |
| Number | Poly ID | ID NO: 5 | Allele | Allele |
| 1 | 711094203 | 1001 | T | C |
| 2 | 711071938 | 9672 | G | C |
| 3 | 711071941 | 10036 | G | A |
| 4 | 711067311 | 20208 | G | A |
| 5 | 711067300 | 21659 | C | T |
| 6 | 711067330 | 45425 | C | T |
| 7 | 711067341 | 45460 | G | C |
| 8 | 711071897 | 48490 | G | T |
| 9 | 710904614 | 54113 | A | G |
| 10 | 711093394 | 55208 | A | T |

TABLE A-26
Polymorphic Sites in the FOS Gene

| | | Position | | |
|---|---|---|---|---|
| PS | | in SEQ | Reference | Variant |
| Number | Poly ID | ID NO: 6 | Allele | Allele |
| 1 | 637600079 | 1001 | C | T |
| 2 | 631698600 | 4876 | T | C |
| 3 | 631698669 | 6762 | G | C |
| 4 | 710974724 | 9125 | G | A |
| 5 | 711093638 | 9569 | T | C |
| 6 | 711094752 | 9871 | G | T |

TABLE A-27
Polymorphic Sites in the DTNBP1 Gene

| | | Position | | |
|---|---|---|---|---|
| PS | | in SEQ | Reference | Variant |
| Number | Poly ID | ID NO: 7 | Allele | Allele |
| 1 | 636094934 | 561 | T | G |
| 2 | 636094940 | 1758 | C | T |
| 3 | 636094950 | 3078 | C | T |
| 4 | 636094952 | 3408 | G | A |
| 5 | 711022632 | 15415 | T | C |
| 6 | 636094969 | 26657 | G | C |
| 7 | 711022607 | 28324 | G | A |
| 8 | 711022598 | 29448 | G | T |
| 9 | 636094987 | 38573 | C | T |
| 10 | 711022507 | 53395 | G | C |
| 11 | 711022388 | 54311 | C | T |
| 12 | 711022397 | 63525 | A | G |
| 13 | 711022513 | 64195 | A | G |
| 14 | 711022368 | 107997 | G | A |
| 15 | 711022453 | 119940 | C | T |
| 16 | 636094993 | 120477 | G | A |
| 17 | 711022525 | 121616 | T | G |
| 18 | 711022353 | 122606 | G | A |
| 19 | 711022531 | 125843 | G | A |
| 20 | 711022582 | 127793 | G | A |
| 21 | 711022483 | 128347 | A | G |
| 22 | 711022348 | 129053 | G | A |
| 23 | 711166842 | 129549 | G | A |
| 24 | 711022342 | 130762 | C | T |

TABLE A-28
Polymorphic Sites in the GABRG3 Gene

| | | Position | | |
|---|---|---|---|---|
| PS | | in SEQ | Reference | Variant |
| Number | Poly ID¹ | ID NO: 8 | Allele | Allele |
| 1 | 710975524 | 632 | G | A |
| 2 | 710975811 | 3472 | C | G |
| 3 | 710975786 | 3745 | T | C |
| 4 | 710975711 | 10975 | A | G |
| 5 | 710975693 | 15371 | T | C |
| 6 | 710975672 | 26644 | G | C |
| 7 | 710975675 | 27987 | C | T |
| 8 | 710975394 | 58104 | C | T |
| 9 | 710975741 | 65440 | A | G |
| 10 | 711106453 | 71334 | C | G |
| 11 | 710975501 | 88634 | C | T |
| 12 | 711106510 | 89236 | T | C |
| 13 | 711106601 | 109805 | G | A |

TABLE A-29
Polymorphic Sites in the GRIA4 Gene

| | | Position | | |
|---|---|---|---|---|
| PS | | in SEQ | Reference | Variant |
| Number | Poly ID | ID NO: 9 | Allele | Allele |
| 1 | 710895723 | 319 | G | A |
| 2 | 710896154 | 1773 | C | A |
| 3 | 711075244 | 6531 | G | C |
| 4 | 710896109 | 7053 | C | T |
| 5 | 710895742 | 9459 | C | T |
| 6 | 710991292 | 9476 | C | T |
| 7 | 710895901 | 10399 | A | T |
| 8 | 710895857 | 37646 | G | A |
| 9 | 710895878 | 48580 | A | G |
| 10 | 710895937 | 48899 | T | C |
| 11 | 710895727 | 53344 | C | T |
| 12 | 710991328 | 54844 | G | T |
| 13 | 710895847 | 64853 | T | C |
| 14 | 710896120 | 65601 | A | G |
| 15 | 710896024 | 69274 | T | C |
| 16 | 710895714 | 69431 | T | C |
| 17 | 710895481 | 74432 | A | G |
| 18 | 710895370 | 74502 | G | T |
| 19 | 710895581 | 79472 | T | C |
| 20 | 710896189 | 79527 | T | C |
| 21 | 711082103 | 82663 | G | C |
| 22 | 710895614 | 86802 | T | C |
| 23 | 710896293 | 87205 | G | A |
| 24 | 710895821 | 97223 | C | T |
| 25 | 710896163 | 109718 | T | C |
| 26 | 710895391 | 10606 | A | G |
| 27 | 710991405 | 110803 | G | C |
| 28 | 710895625 | 111346 | A | G |
| 29 | 710895748 | 113316 | A | T |
| 30 | 711075135 | 114730 | A | G |
| 31 | 710896264 | 114822 | G | C |
| 32 | 710895423 | 116449 | A | G |
| 33 | 710896083 | 118530 | C | T |
| 34 | 710895549 | 118881 | A | G |
| 35 | 710895358 | 119398 | C | A |
| 36 | 711075151 | 120310 | T | C |
| 37 | 710991378 | 120753 | C | T |
| 38 | 710895577 | 124233 | T | C |

TABLE A-30
Polymorphic Sites in the LAMA4 Gene

| | | Position | | |
|---|---|---|---|---|
| | | in SEQ | | |
| PS | | ID | Reference | Variant |
| Number | Poly ID | NO: 10 | Allele | Allele |
| 1 | 711078647 | 365 | A | G |
| 2 | 711078281 | 8622 | G | A |
| 3 | 711010712 | 20526 | T | C |
| 4 | 711078147 | 23348 | T | C |
| 5 | 711078547 | 27327 | G | C |
| 6 | 710991918 | 28940 | G | A |
| 7 | 711078371 | 31804 | G | A |
| 8 | 711078573 | 33702 | A | G |
| 9 | 711078198 | 40082 | A | T |
| 10 | 711078261 | 64952 | G | A |
| 11 | 710991840 | 70308 | C | T |

TABLE A-31
Polymorphic Sites in the MAPK1 Gene

| | | Position | | |
|---|---|---|---|---|
| | | in SEQ | | |
| PS | | ID | Reference | Variant |
| Number | Poly ID | NO: 11 | Allele | Allele |
| 1 | 711010538 | 530 | A | G |
| 2 | 711010648 | 8888 | T | G |
| 3 | 710977682 | 13969 | A | G |
| 4 | 711010526 | 19424 | G | A |
| 5 | 710977700 | 24336 | G | T |
| 6 | 711010594 | 26316 | C | T |
| 7 | 710901334 | 52242 | C | G |
| 8 | 710977679 | 56838 | A | G |
| 9 | 710977688 | 68100 | T | C |
| 10 | 710977685 | 69398 | A | G |
| 11 | 710901146 | 83374 | A | C |
| 12 | 710977697 | 92350 | A | G |
| 13 | 711093563 | 103191 | T | C |
| 14 | 711094789 | 103872 | T | C |

TABLE A-32
Polymorphic Sites in the NPY1R Gene

| | | Position | | |
|---|---|---|---|---|
| | | in SEQ | | |
| PS | | ID | Reference | Variant |
| Number | Poly ID | NO: 12 | Allele | Allele |
| 1 | 710980291 | 1001 | T | C |
| 2 | 711060972 | 1080 | G | A |
| 3 | 711060985 | 1615 | T | A |
| 4 | 711018483 | 2307 | T | A |
| 5 | 710988318 | 3314 | G | T |
| 6 | 711018474 | 4924 | A | G |
| 7 | 711018480 | 6939 | T | C |
| 8 | 711093951 | 14354 | T | A |

TABLE A-33
Polymorphic Sites in the OPRD1 Gene

| | | Position | | |
|---|---|---|---|---|
| | | in SEQ | | |
| PS | | ID | Reference | Variant |
| Number | Poly ID | NO: 13 | Allele | Allele |
| 1 | 711018330 | 2928 | G | A |
| 2 | 644381289 | 4327 | G | A |
| 3 | 644381297 | 8360 | C | T |
| 4 | 710967627 | 14417 | T | C |
| 5 | 644381300 | 15016 | A | G |
| 6 | 710967614 | 17427 | T | C |
| 7 | 710967622 | 25637 | G | A |
| 8 | 644381311 | 25740 | A | G |
| 9 | 710967634 | 27754 | G | A |
| 10 | 711018389 | 38224 | C | T |
| 11 | 711018283 | 39385 | A | G |
| 12 | 644381334 | 42096 | A | T |
| 13 | 711018386 | 44697 | A | G |
| 14 | 644381366 | 53568 | T | A |

TABLE A-34
Polymorphic Sites in the OPRM1 Gene

| | | Position | | |
|---|---|---|---|---|
| | | in SEQ | | |
| PS | | ID | Reference | Variant |
| Number | Poly ID | NO: 14 | Allele | Allele |
| 1 | 710914515 | 4010 | C | T |
| 2 | 710914559 | 8702 | A | C |
| 3 | 710914957 | 9409 | T | C |
| 4 | 710914660 | 15715 | C | A |
| 5 | 710915205 | 22359 | G | A |
| 6 | 711070902 | 48590 | C | T |
| 7 | 710914944 | 52627 | G | C |
| 8 | 711070771 | 55186 | A | G |
| 9 | 710992027 | 60535 | T | G |
| 10 | 710914868 | 64174 | C | T |
| 11 | 710914921 | 64943 | A | G |
| 12 | 710992019 | 77336 | C | A |
| 13 | 711070941 | 80139 | G | T |
| 14 | 710991990 | 82587 | A | G |
| 15 | 710992022 | 83298 | G | T |
| 16 | 711070854 | 90472 | C | A |
| 17 | 710992030 | 96810 | C | T |
| 18 | 711070814 | 97661 | T | A |
| 19 | 711068853 | 99573 | A | C |
| 20 | 711067925 | 123768 | T | G |
| 21 | 711069371 | 126262 | G | A |
| 22 | 711067565 | 127937 | T | A |
| 23 | 711070639 | 128043 | C | T |
| 24 | 711067595 | 152156 | G | C |
| 25 | 711070720 | 159260 | T | G |
| 26 | 711067585 | 160222 | G | T |
| 27 | 711067568 | 165085 | T | G |
| 28 | 711067723 | 169236 | G | C |
| 29 | 711067764 | 173459 | A | C |
| 30 | 711070746 | 178716 | C | A |
| 31 | 711067767 | 178981 | A | G |
| 32 | 711067910 | 179035 | C | T |
| 33 | 711067730 | 182068 | G | T |
| 34 | 711067733 | 182243 | A | G |
| 35 | 711070642 | 183113 | A | G |
| 36 | 711070513 | 190771 | T | C |
| 37 | 711067627 | 205957 | G | A |
| 38 | 711070117 | 207984 | C | A |
| 39 | 711067848 | 211170 | C | T |
| 40 | 711094157 | 211273 | G | A |
| 41 | 711067624 | 211885 | G | A |

TABLE A-35
Polymorphic Sites in the PER3 Gene

| | | Position | | |
|---|---|---|---|---|
| | | in SEQ | | |
| PS | | ID | Reference | Variant |
| Number | Poly ID | NO: 15 | Allele | Allele |
| 1 | 20917924 | 2730 | T | C |
| 2 | 710981364 | 3562 | A | C |
| 3 | 710981377 | 5592 | G | A |
| 4 | 710981361 | 7097 | C | T |
| 5 | 710981355 | 7253 | C | T |
| 6 | 710981371 | 7933 | C | T |
| 7 | 634127186 | 9954 | T | C |
| 8 | 613171908 | 25760 | T | C |
| 9 | 711018146 | 28884 | C | T |
| 10 | 613171917 | 36203 | G | A |
| 11 | 710981374 | 36662 | C | T |
| 12 | 711205161 | 37718 | T | G |
| 13 | 710981358 | 41615 | T | C |
| 14 | 20939499 | 43459 | G | A |
| 15 | 25594125 | 44614 | C | G |
| 16 | 25594117 | 44961 | A | G |
| 17 | 613113405 | 45250 | A | G |
| 18 | 710981380 | 45473 | C | T |
| 19 | 711204131 | 46839 | C | G |
| 20 | 25598753 | 46976 | C | T |
| 21 | 711204289 | 46991 | G | A |
| 22 | 711204136 | 47009 | G | T |
| 23 | 711204126 | 47099 | A | G |
| 24 | 711205104 | 47142 | — | GCTCTGT |
| | | | | CCACAGG |
| | | | | ATCGCCT |
| | | | | CCCATGA |
| | | | | AGAATCC |
| | | | | ATCCCAT |
| | | | | CTTACTG |
| | | | | CCAGC |
| 25 | 711018190 | 47992 | G | C |
| 26 | 711018193 | 48119 | A | T |
| 27 | 20931073 | 54169 | A | G |
| 28 | 634127175 | 63044 | G | T |
| 29 | 711061797 | 65924 | G | A |

TABLE A-36
Polymorphic Sites in the PLCB1 Gene

| | | Position | | |
|---|---|---|---|---|
| | | in SEQ | | |
| PS | | ID | Reference | Variant |
| Number | Poly ID | NO: 16 | Allele | Allele |
| 1 | 710981419 | 432 | G | A |
| 2 | 710981416 | 531 | A | T |
| 3 | 710981880 | 12021 | G | C |
| 4 | 710981792 | 18957 | C | G |
| 5 | 710981825 | 23011 | A | G |
| 6 | 710982077 | 23552 | G | A |
| 7 | 710981556 | 24915 | G | A |
| 8 | 710981911 | 27468 | C | T |
| 9 | 710981916 | 27542 | G | T |
| 10 | 710981919 | 27665 | A | T |
| 11 | 710982023 | 28226 | A | G |
| 12 | 710982026 | 28261 | G | C |
| 13 | 710981922 | 29155 | C | T |
| 14 | 710981666 | 30747 | G | A |
| 15 | 710981857 | 34155 | A | G |
| 16 | 710982080 | 35587 | T | C |
| 17 | 710982009 | 37025 | G | T |

TABLE A-37
Polymorphic Sites in the PSMD1 Gene

| | | Position | | |
|---|---|---|---|---|
| | | in SEQ | | |
| PS | | ID | Reference | Variant |
| Number | Poly ID | NO: 17 | Allele | Allele |
| 1 | 711094697 | 1688 | C | T |
| 2 | 710902871 | 23894 | A | G |
| 3 | 711093905 | 25426 | G | A |
| 4 | 710903088 | 30401 | A | G |
| 5 | 711094687 | 51854 | G | T |
| 6 | 711108560 | 60461 | C | A |
| 7 | 711086956 | 63711 | A | G |
| 8 | 711108574 | 64926 | A | G |
| 9 | 711086951 | 66316 | T | G |
| 10 | 710992176 | 96238 | G | T |

TABLE A-38
Polymorphic Sites in the ABI1 Gene

| PS | | Reference | Variant |
|---|---|---|---|
| Number | Position | Allele | Allele |
| 1 | 201 in SEQ ID NO: 18 | G | A |
| 2 | 201 in SEQ ID NO: 19 | T | G |
| 3 | 201 in SEQ ID NO: 20 | A | G |

TABLE A-39
Polymorphic Sites in the LOC402382 Gene

| PS | | Reference | Variant |
|---|---|---|---|
| Number | Position | Allele | Allele |
| 1 | 201 in SEQ ID NO: 21 | C | T |

TABLE A-40
Polymorphic Sites in the NCALD Gene

| PS | | Reference | Variant |
|---|---|---|---|
| Number | Position | Allele | Allele |
| 1 | 201 in SEQ ID NO: 22 | A | G |

What is claimed is:

1. A method of predicting whether an individual will respond to an antidepressant, the method comprising:
(a) determining the presence or absence in the individual of a genetic marker in any of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes that is associated with response to the antidepressant; and
(b) making a prediction based on the results of the determining step,
wherein if the marker is present, then the prediction is that the individual is likely to respond better to an antidepressant than an individual in whom the marker is absent, and if the marker is absent, then the prediction is that the individual is less likely to respond better to an antidepressant than an individual in whom the marker is present.

2. The method of claim 1, wherein the antidepressant is selected from the group consisting of a selective serotonin reuptake inhibitor, a serotonin-norepinephrine reuptake inhibitor, a norepinephrine reuptake inhibitor, a tricyclic antidepressant, and a monoamine oxidase inhibitor.

3. The method of claim 2, wherein the antidepressant is a selective serotonin reuptake inhibitor.

4. The method of claim 3, wherein the selective serotonin reuptake inhibitor is vilazodone.

5. A method for treating depression in an individual, the method comprising:
(a) determining the presence or absence in the individual of a genetic marker in any of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes that is associated with response to an antidepressant; and
(b) making a treatment decision based on the results of the determining step,
wherein if the marker is present, then the decision is to prescribe to the individual the lowest approved dose of the antidepressant, and if the marker is absent, then the decision is to either prescribe to the individual the antidepressant at a dose that is higher than the lowest approved dose, or a therapy not including the antidepressant that is effective in treating depression.

6. The method of claim 5, wherein the antidepressant is selected from the group consisting of a selective serotonin reuptake inhibitor, a serotonin-norepinephrine reuptake inhibitor, a norepinephrine reuptake inhibitor, a tricyclic antidepressant, and a monoamine oxidase inhibitor.

7. The method of claim 6, wherein the antidepressant is a selective serotonin reuptake inhibitor.

8. The method of claim 7, wherein the selective serotonin reuptake inhibitor is vilazodone.

9. A kit for detecting a genetic marker in any of the ACE, ATP5C3, BCL2L1, CYP2C9, DRD3, FOS, DTNBP1, GABRG3, GRIA4, LAMA4, MAPK1, NPY1R, OPRD1, OPRM1, PER3, PLCB1, PSMD1, ABI1, LOC402382, and NCALD genes that is associated with antidepressant response, the kit comprising a set of oligonucleotides designed for identifying each of the alleles at each polymorphic site (PS) in the marker.

10. The kit of claim 9, wherein the set of oligonucleotides comprises an allele-specific oligonucleotide (ASO) probe for each allele at each PS.

11. The kit of claim 9, wherein the set of oligonucleotides comprises a primer-extension oligonucleotide for each PS.

12. The kit of claim 9, wherein the antidepressant is selected from the group consisting of a selective serotonin reuptake inhibitor, a serotonin-norepinephrine reuptake inhibitor, a norepinephrine reuptake inhibitor, a tricyclic antidepressant, and a monoamine oxidase inhibitor.

13. The kit of claim 12, wherein the antidepressant is a selective serotonin reuptake inhibitor.

14. The kit of claim 13, wherein the selective serotonin reuptake inhibitor is vilazodone.

15. The kit of claim 10, wherein the antidepressant is selected from the group consisting of a selective serotonin reuptake inhibitor, a serotonin-norepinephrine reuptake inhibitor, a norepinephrine reuptake inhibitor, a tricyclic antidepressant, and a monoamine oxidase inhibitor.

16. The kit of claim 15, wherein the antidepressant is a selective serotonin reuptake inhibitor.

17. The kit of claim 16, wherein the selective serotonin reuptake inhibitor is vilazodone.

18. The kit of claim 11, wherein the antidepressant is selected from the group consisting of a selective serotonin reuptake inhibitor, a serotonin-norepinephrine reuptake inhibitor, a norepinephrine reuptake inhibitor, a tricyclic antidepressant, and a monoamine oxidase inhibitor.

19. The kit of claim 18, wherein the antidepressant is a selective serotonin reuptake inhibitor.

20. The kit of claim 19, wherein the selective serotonin reuptake inhibitor is vilazodone.
